(19) United States
(12) Patent Application Publication
(10) Pub. No.: US 2005/0150002 A1
(43) Pub. Date: Jul. 7, 2005

(54) Novel carotenoid hydroxylases for use in engineering carotenoid metabolism in plants

(76) Inventors: Dean DellaPenna, Williamston, MI (US); Li Tian, Ardmore, OK (US); Joonyul Kim, Inje (KR)

Correspondence Address: MEDLEN & CARROLL, LLP, Suite 350, 101 Howard Street, San Francisco, CA, 94105, US

(21) Appl. No.: 10/751,235

(22) Filed: Jan. 2, 2004

(51) Int. Cl. A01H 1/00; C12N 15/82; C12N 9/02; C12N 5/04; C12P 23/00

(52) U.S. Cl. 800/278; 435/468; 435/419; 435/189; 800/282; 435/67

(57) ABSTRACT

The present invention relates to genes, proteins and methods comprising carotenoid monooxygenases in the cytochrome P450 family. In a preferred embodiment, the present invention relates to altering carotenoid ratios in plants and microorganisms using LUT1 ε-hydroxylases and/or CYP97A β-hydroxylases.

The present application was funded in part with government support under grant number IBN-0131253 from the National Science Foundation. The government may have certain rights in this invention.

### FIELD OF THE INVENTION

The present invention relates to genes, proteins and methods comprising carotenoid monooxygenases in the cytochrome P450 family. In a preferred embodiment, the present invention relates to altering carotenoid ratios in plants and microorganisms using LUT1 ε-hydroxylases and/or CYP97A β-hydroxylases.

### BACKGROUND

Carotenoids are used for a variety of commercial products ranging from pigments to color foods and cosmetics to dietary supplements in animal and poultry feedstuffs. Plants are a major source of carotenoids such as lutein (bright yellow), zeaxanthin (bright orange) and lycopene (bright red). These three carotenoids are considered potent antioxidants. Lutein and zeaxanthin are believed to prevent many types of diseases including Age-Related Macular Degeneration, while their precursor carotenoids such as lycopene are believed to prevent certain types of cancer.

Plants are the primary sources of carotenoids. However, the amount of any particular carotenoid per plant is low and a steady diet of food items is necessary to provide the full range of dietary carotenoids. However, such foods are unavailable or of limited availability in many populated areas of the world. Production of concentrated carotenoids from wild-type plants is expensive because of the low yields and variability of carotenoid production. Thus, concentrated forms of specific carotenoids are available in limited quantities as expensive dietary supplements. Significant dietary amounts of lutein/zeaxanthin or lycopene are not present in the majority of more ubiquitous crop plants such as peas, barley, soybeans, wheat, rice etc. and certain enzymes necessary for engineering certain types of carotenoids are not currently available.

Therefore, it would be of considerable advantage to our rapidly expanding global population to be able to engineer carotenoid production in a variety of regional food crops to enhance production of specific carotenoid compounds. Finally, there remains a need for transformed plant (or non-plant) species to produce inexpensive sources of specific carotenoids.

### SUMMARY OF THE INVENTION

The present invention relates to genes, proteins and methods comprising carotenoid monooxygenases in the cytochrome P450 family. In a preferred embodiment, the present invention relates to altering carotenoid ratios in plants and microorganisms using LUT1 ε-hydroxylases and/or CYP97A β-hydroxylases.

The present invention is not limited to any particular sequence encoding a protein having monooxygenase, β-ring and/or ε-ring hydroxylase activities. In some embodiments, the invention provides an expression vector comprising a nucleic acid sequence encoding a polypeptide at least 40% identical to SEQ ID NO: 1, wherein the nucleic acid sequence encodes a protein having monooxygenase activity. In other embodiments, the present invention provides an expression vector comprising nucleotide sequences encoding a polypeptide that is at least 50%, 60%, 70%, 80%, 90%, 95% (or more) identical to any of SEQ ID NOs: 1-4, 16-21, 33-39, 49-52 and 56. In some embodiments the nucleic acid sequence encodes a protein having monooxygenase activity. In some embodiments the nucleic acid sequence encodes a protein having hydroxylase activity. In some embodiments, the nucleic acid sequence encodes a protein having β-ring hydroxylase activity. In some embodiments, the nucleic acid sequence encodes a protein having ε-ring hydroxylase activity. In other embodiments, the proteins with ε-ring hydroxylase activity further comprise β-ring hydroxylase activity.

In still other embodiments, the nucleic acid sequence further comprises a sequence encoding a cytochrome P450 molecular oxygen binding pocket conserved consensus amino acid motif corresponding to SEQ ID NO: 12. In other embodiments, the nucleic acid sequence further comprises a sequence encoding a conserved transmembrane domain sequence corresponding to SEQ ID NO: 10. In further embodiments, the nucleic acid sequence further comprises a sequence encoding a conserved consensus cysteine motif in P450 molecules corresponding to SEQ ID NO: 14. In other embodiments, the nucleic acid sequence further comprises a sequence encoding a LUT1 conserved consensus cysteine amino acid motif corresponding to SEQ ID NO: 15. In still further embodiments, the nucleic acid sequence further comprises a sequence encoding a conserved N-terminal transit peptide for chloroplast-targeting corresponding to SEQ ID NO:11.

In still other embodiments, the nucleic acid sequence encoding a polypeptide at least 40% identical to SEQ ID NO: 1 is selected from the group consisting of SEQ ID NOs: 1-4, 16-21, 33-39, 49-52, and 56. In further embodiments, the nucleic acid sequence is selected from the group consisting of SEQ ID NOs: 5-9, 22-27, 40-48, 53-55, 57 and 58. Accordingly, in some embodiments the present invention provides expression vectors comprising nucleic acid sequences at least 40% identical to any one of SEQ ID NOs: 5-9, 22-27, 40-48, 53-55, 57 and 58. In further embodiments, the nucleic acid sequence is at least 40%, 60%, 70%, 80%, 90%, 95% (or more) identical to any of SEQ ID NOs: 5-9, 22-27, 40-48, 53-55, 57 and 58.

The present invention is not limited to any particular type of vector. Indeed, a variety of vectors are contemplated. In some embodiments, the expression vector is a eukaryotic vector. In further embodiments, the eukaryotic vector is a plant vector. In still further embodiments, the plant vector is a T-DNA vector. In other embodiments, the expression vector is a prokaryotic vector.

In some embodiments, the present invention provides nucleic acid sequences encoding a polypeptide at least 40% identical to SEQ ID NO: 1 operably linked to an heterologous promoter, wherein the nucleic acid sequence encodes a polypeptide having hydroxylase activity. The present invention is not limited to any particular type of hydroxylase activity. In some embodiments, hydroxylase activity is ε-ring hydroxylase activity. In some embodiments, hydroxylase activity is β-ring hydroxylase activity. It is not meant to limit the proteins of the present invention to one type of hydroxylase activity. In some embodiments, hydroxylase activity is dual ε-ring and β-ring hydroxylase activity. Accordingly in other embodiments, the polypeptide is at least 50%, 60%, 70%, 80%, 90%, 95% (or more) identical to any of SEQ ID NOs: 1-4, 16-21, 33-39, 49-52 and 56. The present invention is not limited to any particular type of promoter. Indeed, the use of a variety of promoters is contemplated. In some embodiments, the promoter is a eukaryotic promoter. In further embodiments, the eukaryotic promoter is active in a plant.

In other embodiments, the present invention provides an expression vector, comprising a first nucleic acid sequence encoding a nucleic acid product that interferes with the expression of a second nucleic acid sequence encoding a polypeptide at least 40% identical to SEQ ID NO: 1. Accordingly in other embodiments, the polypeptide is at least 50%, 60%, 70%, 80%, 90%, 95% (or more) identical to any of SEQ ID NOs: 1-4, 16-21, 33-39, 49-52 and 56. The present invention is not limited to the any particular interfering nucleic acid product. Indeed, the use of a variety of such products is contemplated. In some embodiments, the nucleic acid product that interferes is an antisense sequence. In other embodiments, the nucleic acid product that interferes is a dsRNA that mediates RNA interference.

In further embodiments, the present invention provides a transgenic plant comprising a nucleic acid sequence encoding a polypeptide at least 40% identical to SEQ ID NO: 1, wherein said nucleic acid sequence encodes a protein having hydroxylase activity, and wherein the nucleic acid sequence is heterologous to the plant. Accordingly in other embodiments, the polypeptide is at least 50%, 60%, 70%, 80%, 90%, 95% (or more) identical to any of SEQ ID NOs: 1-4, 16-21, 33-39, 49-52 and 56. The present invention is not limited to any particular transgenic plant. In some embodiments, transgenic plants are crop plants. Indeed, a variety of transgenic plants are contemplated, including, but not limited to one or more of the following: Arabidopsis thaliana, Helianthus annuus, Lycopersicon esculentum, Oryza sativa, Zea mays, Hordeum vulgare, Triticum aestivum, Glycine max, Pisum sativum, Chlamydomonas reinhardtii; one or more of Tagetes (marigolds), one or more of asterids, one or more of Chlorophyta, one or more of the following families Brassicaceae, Poaceae, Fabaceae, Asteraceae, Solanaceae and Volvocaceae; one or more of core eudicots, one or more members of Viridiplantae.

In some embodiments, the present invention provides a transgenic plant cell comprising a nucleic acid sequence encoding a polypeptide at least 40% identical to SEQ ID NO: 1, wherein the nucleic acid sequence encodes a protein having hydroxylase activity, and wherein the nucleic acid sequence is heterologous to the plant cell. Accordingly in other embodiments, the polypeptide is at least 50%, 60%, 70%, 80%, 90%, 95% (or more) identical to any of SEQ ID NOs: 1-4, 16-21, 33-39, 49-52 and 56.

In other embodiments, the present invention provides a transgenic plant seed comprising a nucleic acid sequence encoding a protein at least 40% identical to SEQ ID NO: 1, wherein the nucleic acid sequence encodes a polypeptide having hydroxylase activity, and wherein the nucleic acid sequence is heterologous to the plant seed. Accordingly in other embodiments, the polypeptide is at least 50%, 60%, 70%, 80%, 90%, 95% (or more) identical to any of SEQ ID NOs: 1-4, 16-21, 33-39, 49-52 and 56.

In further embodiments, the invention provides a transgenic plant comprising a nucleic acid encoding a protein at least 40% identical to SEQ ID NO: 1 operably linked to a promoter, wherein the nucleic acid sequence encodes a polypeptide having monooxygenase and/or β or ε-ring hydroxylase activity. Accordingly in other embodiments, the polypeptide is at least 50%, 60%, 70%, 80%, 90%, 95% (or more) identical to any of SEQ ID NOs: 1-4, 16-21, 33-39, 49-52 and 56.

In some embodiments, the present invention provides methods for altering the phenotype of a plant, comprising: a) providing; i) an expression vector as described in detail above, and ii) plant tissue; and b) transfecting the plant tissue with the vector under conditions that alter the phenotype of a plant.

In other embodiments, the present invention provides methods for altering carotenoid ratios, comprising: a) providing a vector construct comprising a nucleic acid encoding a polypeptide at least 40% identical to SEQ ID NO: 1, wherein said nucleic acid sequence encodes a protein having ε-ring hydroxylase activity; and b) producing a plant comprising the vector, wherein the plant exhibits altered carotenoid ratios. Accordingly in other embodiments, the polypeptide is at least 50%, 60%, 70%, 80%, 90%, 95% (or more) identical to any of SEQ ID NOs: 1-4, 16-21, 33-39, 49-52 and 56.

In further embodiments, the present invention provides methods for altering the carotenoid production of a plant, comprising: a) providing; i) an expression vector comprising a nucleic acid encoding a polypeptide at least 40% identical to SEQ ID NO: 1, wherein the nucleic acid sequence encodes a protein having ε-ring hydroxylase activity, and, and ii) plant tissue; and b) introducing the vector into the plant tissue under conditions such that the protein encoded by the nucleic acid sequence is expressed so that the plant tissue exhibits altered carotenoid ratios. Accordingly in other embodiments, the polypeptide is at least 50%, 60%, 70%, 80%, 90%, 95% (or more) identical to any of SEQ ID NOs: 1-4, 16-21, 33-39, 49-52 and 56.

In further embodiments, the invention provides a method for producing lutein, comprising: a) providing a transgenic host cell comprising a heterologous nucleic acid sequence, wherein the heterologous nucleic acid sequence encodes a polypeptide at least 40% identical to SEQ ID NO: 1, under conditions sufficient for expression of the encoded protein; and b) culturing the transgenic host cell under conditions such that lutein is produced. Accordingly in other embodiments, the polypeptide is at least 50%, 60%, 70%, 80%, 90%, 95% (or more) identical to any of SEQ ID NOs: 1-4, 16-21, 33-39, 49-52 and 56. The present invention is not limited to the use of any particular type of host cell. Indeed, a variety of host cells are contemplated, including, but not limited to the one or more of the following: Skeletonema, a Skeletonemataceae, a Coscinodiscophyceae (centric diatoms), a bacillariophyta (diatoms), a stramenopiles (heterokonts), a Eukaryota (eucaryotes), an Enterobacteriaceae, an Enterobacteriales, a Gammaproteobacteria, a Proteobacteria or a bacterium.

In further embodiments, the present invention provides a method for increasing the levels of non-hydroxylated carotenes in a plant tissue, comprising: a) providing a transgenic plant tissue comprising a heterologous nucleic acid sequence, wherein the heterologous nucleic acid sequence encodes a polypeptide at least 40% identical to SEQ ID NO: 1, under conditions sufficient for expression of the encoded protein; and b) culturing the transgenic plant tissue under conditions for increasing the levels of non-hydroxylated carotenes in the plant tissue. Accordingly in other embodiments, the polypeptide is at least 50%, 60%, 70%, 80%, 90%, 95% (or more) identical to any of SEQ ID NOs: 1-4, 16-21, 33-39, 49-52 and 56. The present invention is not limited to increasing any particular type of non-hydroxylated carotenes. Indeed, increasing a wide variety of non-hydroxylated carotenes is contemplated. In one embodiment, alpha non-hydroxylated carotenes are increased. In another embodiment, beta non-hydroxylated carotenes are increased. In yet another embodiment, both alpha and beta non-hydroxylated carotenes are increased. In a further embodiment, any non-hydroxylated carotene is increased.

### DESCRIPTION OF THE FIGURES

FIG. 1. shows exemplary embodiments in which biosynthetic steps leading to lutein and zeaxanthin from alpha and beta carotene respectively are blocked by the b1 (β-hydroxylase 1), b2 (β-hydroxylase 2), and lut1 (ε-hydroxylase) mutations as indicated.

FIG. 2. shows exemplary embodiments which demonstrates (A) positional cloning of the LUT1 locus showing recombinants as indicated for specific SSLP markers across the interval and the position of chloroplast-targeted proteins are indicated by dashed arrows, (B) overview of the intron-exon organization of LUT1 and the locations of the lut1-1 and lut1-3 mutations, and (C) Deduced amino acid sequence of LUT1. The cleavage site of the putative chloroplast targeting sequence is indicated by an arrow and the single predicted transmembrane domain is shaded in black. The conserved cytochrome P450 molecular oxygen binding pocket and the cysteine motif are indicated by single and double underlines, respectively, and the conserved Thr by an asterisk.

FIG. 3. shows exemplary embodiments which demonstrates HPLC elution profiles of total leaf carotenoid extracts from (A) wild type, (B) lut1-1, (C) lut1-3, and (D) lut1-1 transformed with pMLBART-At3g53130. Peaks correspond to: N, neoxanthin; V, violaxanthin; A, antheraxanthin; L, lutein; Z, zeaxanthin; b, chlorophyll b; zei, zeinoxanthin; a, chlorophyll a; B, β-carotene.

FIG. 4. shows exemplary embodiments that demonstrate the relative wild type or mutant LUT1 transcript level detected in each genotype by Real-Time PCR (refer to Materials and Methods). The relative quantity of the LUT1 mRNA has been corrected with EF1α. Data shown are means+SD (n=6).

FIG. 5. shows exemplary embodiments that demonstrate phylogenetic analysis of CYP97C and CYP97B sequences. A rooted neighbor-joining tree was constructed using the fatty acid ω-hydroxylase (CYP86A8) from Arabidopsis thaliana as an outgroup. Bootstrap values are indicated adjacent to the branches. Accession numbers for the sequences used are listed with these sequences.

FIG. 6. shows exemplary embodiments that demonstrate the substrates and proposed mechanisms of carotenoid hydroxylation reactions. (A) The hydroxylation reactions of β- and ε-rings. R, polyene chain. (B) 3-D structures of α- and β-carotene hydroxylation substrates. The left rings of both molecules are β-rings while the right rings are β- and ε-rings, respectively, for β- and α-carotene.

FIG. 7. shows exemplary embodiments that demonstrate an overview of the intron-exon organization of CYP97A3 (Arabidopsis) and the locations of a functional single knockout mutant (SALK_{—}116660).

FIG. 8. shows exemplary embodiments that demonstrate phylogenetic analysis of CYP97A and CYP97C sequences. A rooted neighbor-joining tree was constructed using the fatty acid ω-hydroxylase (CYP86A8) from Arabidopsis thaliana as an outgroup. Bootstrap values are indicated adjacent to the branches. Accession numbers for the sequences used are listed with these sequences.

FIG. 9. shows exemplary embodiments that demonstrate alignments of CYP97 sequences.

FIG. 10. shows exemplary embodiments that demonstrate sequence similarities of CYP97A and CYP97C sequences. A rooted neighbor-joining tree was constructed using CYP97B from Arabidopsis thaliana as an outgroup. Bootstrap values are indicated adjacent to the branches.

FIG. 11. shows exemplary embodiments that demonstrate phylogenetic analysis of CYP97A and CYP97C sequences.

FIG. 12. shows exemplary embodiments that demonstrate plasmid constructs used in the present invention.

FIG. 13. shows exemplary embodiments as Table 1 that demonstrate β-Xanthophyll production and β-ring hydroxylation in leaf tissue of wild type and carotenoid hydroxylase mutants.

FIG. 14. shows exemplary embodiments of carotenoid analysis of CYP97C1 single knockout.

FIG. 15. shows exemplary embodiments of carotenoid analysis of CYP97A3 single knockout.

FIG. 16. shows exemplary embodiments of carotenoid analysis of b1b2CYP97C1 triple knockout.

FIG. 17. shows exemplary embodiments of carotenoid analysis that demonstrates alterations in carotenoid production for a CYP97C1 single knockout, a CYP97A3 single knockout, and a b1b2CYP97C1 triple knockout: neo, neoxanthin; vio, violaxanthin; ant, antheraxanthin; lut, lutein; zea, zeaxanthin; zei, zeinoxanthin; β-car, β-carotene

FIG. 18. SEQ ID NO: 1: shows a portion of an amino acid sequence for CYP97C1 Arabidopsis thaliana (Brassicaceae; thale cress). SEQ ID NO: 2: shows a portion of an amino acid sequence for CYP97A3 Arabidopsis thaliana (Brassicaceae; thale cress). SEQ ID NO: 3: shows a portion of an amino acid sequence for Arabidopsis thaliana CYP97B (Brassicaceae; thale cress).

FIGS. 19a and b. SEQ ID NO: 4: shows an amino acid sequence for CYP97C1 Arabidopsis thaliana (Brassicaceae; thale cress). SEQ ID NO: 5: shows a LUT1 cDNA sequence. SEQ ID NO: 6: shows a DNA sequence including LUT1 (At3g53130) genomic sequence plus 1000 bp upstream from the start codon and 700 bp downstream from the stop codon in the Arabidopsis Columbia ecotype (background for lut1-1 and lut1-2 mutations). This sequence was subcloned into pMLBART vector and complemented lut1-1 mutation.

FIG. 20. SEQ ID NO: 7: shows a portion of the genomic nucleotide sequence of mutant Arabidopsis thaliana LUT1-1 (lut1-1) (Brassicaceae; thale cress).

FIG. 21. SEQ ID NO: 8: shows an upstream region nucleotide sequence of leaky mutant Arabidopsis thaliana LUT1-2 (lut1-2) (Brassicaceae; thale cress). SEQ ID NO: 9: shows an a cDNA nucleotide sequence of knockout mutant Arabidopsis thaliana LUT1-3 sequence (lut1-3) (Brassicaceae; thale cress).

FIG. 22. SEQ ID NO: 10: shows an amino acid sequence for a conserved transmembrane domain. SEQ ID NO: 11: shows an amino acid sequence for a conserved an N-terminal transit peptide for chloroplast-targeting. SEQ ID NO: 12: shows an amino acid sequence for a conserved consensus motif of cytochrome P450 molecular oxygen binding pocket. SEQ ID NO: 13: shows an amino acid sequence for a conserved consensus sequence of cytochrome P450 molecular oxygen binding pocket of an Arabidopsis thaliana (Brassicaceae; thale cress) LUT1 protein. SEQ ID NO: 14: shows an amino acid sequence for a conserved consensus cysteine motif in p450 enzymes. SEQ ID NO: 15: shows an amino acid sequence for a conserved cysteine sequence in Arabidopsis thaliana (Brassicaceae; thale cress) LUT1.

FIG. 23. SEQ ID NO: 16: shows a deduced amino acid sequence for rice CYP97C2 Oryza sativa (Poaceae; grass family) (AAK20054; AK065689, GenBank). SEQ ID NO: 17: shows a full-length deduced amino acid sequence for barley CYP97C Hordeum vulgare (Poaceae; grass family) (extracted from BM816653; BU987393; CA023004; AV835803, GenBank). SEQ ID NO:18: shows an amino acid sequence for wheat CYP97C Triticum aestivum (Poaceae; grass family) (extracted from CA497665; BG906289; CA742365; CA742792, GenBank). SEQ ID NO: 19: shows a deduced amino acid sequence for tomato CYP97C Lycopersicon esculentum (Solanaceae; nightshade family) (BG643819 GenBank). SEQ ID NO: 20: shows a deduced amino acid sequence for maize CYP97C Zea mays (Poaceae; grass family) (BE552887 GenBank). SEQ ID NO: 21: shows a deduced amino acid sequence for sunflower CYP97C Helianthus annuus (Asteraceae; daisy family) (BQ971938 GenBank).

FIGS. 24a and b. SEQ ID NO: 22: shows a full-length cDNA nucleotide sequence for rice CYP97C Oryza sativa (Poaceae; grass family) (AK065689 GenBank). SEQ ID NO: 23: shows a full-length cDNA nucleotide sequence for barley CYP97C Hordeum vulgare (Poaceae; grass family) (extracted from BM816653; BU987393; CA023004; AV835803, GenBank). SEQ ID NO: 24: shows a cDNA nucleotide sequence for wheat CYP97C Triticum aestivum (Poaceae; grass family) (extracted from CA497665; BG906289; CA742365; CA742792, GenBank). SEQ ID NO: 25: shows a portion of a cDNA nucleotide sequence for tomato CYP97C Lycopersicon esculentum (Solanaceae; nightshade family) (BG643819, GenBank). SEQ ID NO: 26: shows a portion of a cDNA nucleotide sequence for maize CYP97C Zea mays (Poaceae; grass family) (BE552887, GenBank). SEQ ID NO: 27: shows a portion of a cDNA nucleotide sequence for sunflower CYP97C Helianthus annuus (Asteraceae; daisy family) (BQ971938, GenBank).

FIG. 25. SEQ ID NO: 28: shows a forward At3g53130 primer. SEQ ID NO: 29: shows a reverse At3g53130 primer. SEQ ID NO: 30: shows a LUT1 TaqMan probe. SEQ ID NO: 31: shows a forward LUT1 primer. SEQ ID NO: 32: shows a reverse LUT1 primer.

FIGS. 26a and b. SEQ ID NO: 33: shows a deduced amino acid sequence for Arabidopsis thaliana CYP97A3 (Brassicaceae; thale cress) (At1g31800; AAL08302, AY058173, GenBank), (TIGR database At1g31800). SEQ ID NO: 34: shows a deduced amino acid sequence for rice CYP97A Oryza sativa (Poaceae; grass family) (AP004028, GenBank). SEQ ID NO: 35: shows a portion deduced amino acid sequence for barley CYP97A Hordeum vulgare (Poaceae; grass family) (extracted from AV939715; AV941342; AV939552; AV939356; CA004011; BJ480615; BJ485000; BJ448041; BJ455787; AV910152; AV938407; AJ477620; AJ477618; AJ477619; AV832622, GenBank). SEQ ID NO: 36: shows a deduced amino acid sequence for soybean CYP97A of Glycine max (Fabaceae; pea family) (EXTRACTED FROM BF425906; BF596805; AW704660; AW704625; BI470164; BQ296458; BM892469; AI938600; AI938382; BU544173; BI471346; CD410775; BF598710; BG154747, GenBank). SEQ ID NO: 37: shows a portion of a deduced amino acid sequence for wheat CYP97A Triticum aestivum (Poaceae; grass family) (extracted from BJ234910; CA736787 CA736801; BJ238659; BJ233019; CD882035; GenBank). SEQ ID NO: 38: shows a deduced amino acid sequence for tomato CYP97A Lycopersicon esculentum (Solanaceae; nightshade family) (extracted from CYPAW738390; AI773114; AW737571; BG123929; AW651509; AI773792, GenBank). SEQ ID NO: 39: shows a deduced amino acid sequence for a green alga CYP97A3 homolog of Chlamydomonas reinhardtii (Chlamydomonadaceae; unicellular flagellated green alga) (Scaffold_{—}1399).

FIGS. 27a-g. SEQ ID NO: 40: shows a nucleotide sequence for Arabidopsis thaliana CYP97A (Brassicaceae; thale cress) (AY056446 GenBank). SEQ ID NO: 41: shows a nucleotide sequence for Arabidopsis thaliana CYP97A (Brassicaceae; thale cress) (AY058173 GenBank). SEQ ID NO: 42: shows a portion of a genomic nucleotide sequence for rice CYP97A Oryza sativa (Poaceae; grass family) (AP004028). SEQ ID NO: 43: shows a portion of a genomic nucleotide sequence for rice CYP97A Oryza sativa (Poaceae; grass family) (AP004028). SEQ ID NO: 44: shows a portion of a cDNA nucleotide sequence CYP97A barley Hordeum vulgare (Poaceae; grass family) (extracted from AV939715; AV941342; AV939552; AV939356; CA004011; BJ480615; BJ485000; BJ448041; BJ455787; AV910152; AV938407; AJ477620; AJ477618; AJ477619; AV832622). SEQ ID NO: 45: shows a portion of a cDNA nucleotide sequence Soybean CYP97A of Glycine max (Fabaceae; pea family) (EXTRACTED FROM BF425906; BF596805; AW704660; AW704625; BI470164; BQ296458; BM892469; AI938600; AI938382; BU544173; BI471346; CD410775; BF598710; BG154747). SEQ ID NO: 46: shows a portion of a cDNA nucleotide sequence for CYP97A wheat Triticum aestivum (Poaceae; grass family) (extracted from BJ234910; CA736787; CA736801; BJ238659; BJ233019; CD882035). SEQ ID NO: 47: shows a portion cDNA sequence for CYP97A tomato Lycopersicon esculentum (Solanaceae; nightshade family) (extracted from CYPAW738390; AI773114; AW737571; BG123929; AW651509; AI773792). SEQ ID NO: 48: shows a nucleotide sequence of cDNA for a CYP97A like gene of Chlamydomonas reinhardtii (Chlamydomonadaceae; unicellular flagellated green alga) (CYP97A3 homolog [Scaffold139]).

FIG. 28. SEQ ID NO: 49: shows a deduced amino acid sequence for CYP97B3 in Arabidopsis thaliana (Brassicaceae; thale cress) (CAB10290, TIGR At4g15110). SEQ ID NO: 50: shows a deduced amino acid sequence for CYP97B1 and CYP97A2 of Pisum sativum (Fabaceae; pea family) (CAA89260 GenEMBL Z49263; Q43078). SEQ ID NO: 51: shows a deduced amino acid sequence for CYP97B2 of Glycine max (Fabaceae; pea family) (Genbank AAB94586; GenEMBL AF022457—corrected by author; TC163981 TIGR-Unique Gene Indices). SEQ ID NO: 52: shows a deduced amino acid sequence for CYP97B4 Oryza sativa (japonica cultivar-group) (Poaceae; rice) (EMBL E017117; AE016959, PlaCe database).

FIGS. 29a and b. SEQ ID NO: 53: shows a portion of a deduced mRNA nucleotide sequence of CYP97B3 in Arabidopsis thaliana (Brassicaceae; thale cress) (At4g15110). SEQ ID NO: 54: shows a portion of an mRNA nucleotide sequence of CYP97B1 and CYP97A2 for Pisum sativum (Fabaceae; pea family) (Z49263 GenBank). SEQ ID NO: 55: shows a nucleic acid sequence for soybean CYP97B2 of Glycine Max (Fabaceae; pea family) (AAB94586; AF022457, GenBank).

FIG. 30. SEQ ID NO: 56: shows a deduced amino acid sequence for a novel cytochrome P450 marine diatom in Skeletonema costatum (Skeletonemataceae; centric diatom) (AF459441; AAL73435, GenBank).

FIG. 31. SEQ ID NO: 57: shows a cDNA nucleic acid sequence for a diatom novel cytochrome P450 Skeletonema costatum (Skeletonemataceae; centric diatom) (AF459441 GenBank).

FIG. 32. SEQ ID NO: 58: shows a knockout mutant Arabidopsis thaliana CYP97A3 (Brassicaceae; thale cress).

### DEFINITIONS

To facilitate an understanding of the present invention, a number of terms and phrases as used herein are defined below:

The use of the article “a” or “an” is intended to include one or more.

The terms “cytochrome P450 family” and “cytochrome P450 genes” refers to genes found in all organisms from bacteria to humans. The term “cytochrome P450 protein” refers to proteins that share a common catalytic center, heme with iron coordinated to the thiolate of a conserved cysteine, and a common overall topology and three-dimensional fold (P450terp.swmed.edu/Bills_folder/billhome.htm) (Graham and Peterson, 1999; Werck-Reichhart and Feyereisen, 2000). The term “cytochrome P450 monooxygenase” refers to the ability of the majority of cytochrome P450 proteins to catalyze reactions based on activation of molecular oxygen with insertion of one of its atoms into the substrate and reduction of the other to form water (Mansuy, 1998 ; Werck-Reichhart and Feyereisen, 2000).

The term plant cell “compartments or organelles” is used in its broadest sense. The term includes but is not limited to, the endoplasmic reticulum, Golgi apparatus, trans Golgi network, plastids, sarcoplasmic reticulum, glyoxysomes, mitochondrial, chloroplast, thylakoid membranes and nuclear membranes, and the like.

The term “portion” when used in reference to a protein (as in “a portion of a given protein”) refers to fragments of that protein. The fragments may range in size from four amino acid residues to the entire amino sequence minus one amino acid.

The term “gene” encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region termed “exon” or “expressed regions” or “expressed sequences” interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

The terms “allele” and “alleles” refer to each version of a gene for a same locus that has more than one sequence. For example, there are multiple alleles for eye color at the same locus.

The terms “recessive,” “recessive gene,” and “recessive phenotype” refers to an allele that has a phenotype when two alleles for a certain locus are the same as in “homozygous” or as in “homozygote” and then partially or fully loses that phenotype when paired with a more dominant allele as when two alleles for a certain locus are different as in “heterozygous” or in “heterozygote.” The terms “dominant,” “dominant allele,” and “dominant phenotype” refers to an allele that has an effect to suppress the expression of the other allele in a heterozygous (having one dominant allele and one recessive allele) condition.

The term “heterologous” when used in reference to a gene or nucleic acid refers to a gene that has been manipulated in some way. For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). Heterologous genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed).

The term “nucleic acid sequence,” “nucleotide sequence of interest” or “nucleic acid sequence of interest” refers to any nucleotide sequence (e.g., RNA or DNA), the manipulation of which may be deemed desirable for any reason (e.g., treat disease, confer improved qualities, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

The term “structural” when used in reference to a gene or to a nucleotide or nucleic acid sequence refers to a gene or a nucleotide or nucleic acid sequence whose ultimate expression product is a protein (such as an enzyme or a structural protein), an rRNA, an sRNA, a tRNA, etc.

The term “oligonucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The term “polynucleotide” refers to refers to a molecule comprised of several deoxyribonucleotides or ribonucleotides, and is used interchangeably with oligonucleotide. Typically, oligonucleotide refers to shorter lengths, and polynucleotide refers to longer lengths, of nucleic acid sequences.

The term “an oligonucleotide (or polypeptide) having a nucleotide sequence encoding a gene” or “a nucleic acid sequence encoding” a specified polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers, exogenous promoters, splice junctions, intervening sequences, polyadenylation signals, etc. or a combination of both endogenous and exogenous control elements. The term “exogenous promote”

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “SNP” and “Single Nucleotide Polymorphism” refers to a single base difference found when comparing the same DNA sequence from two different individuals.

The term “partially homologous nucleic acid sequence” refers to a sequence that at least partially inhibits (or competes with) a completely complementary sequence from hybridizing to a target nucleic acid and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence that is completely complementary to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of identity (e.g., less than about 30% identity); in the absence of non-specific binding the probe will not hybridize to the second non-identical target.

The term “substantially homologous” when used in reference to a double-stranded nucleic acid sequence such as a cDNA or genomic clone refers to any probe that can hybridize to either or both strands of the double-stranded nucleic acid sequence under conditions of low to high stringency as described above.

The term “substantially homologous” when used in reference to a single-stranded nucleic acid sequence refers to any probe that can hybridize (i.e., it is the complement of) the single-stranded nucleic acid sequence under conditions of low to high stringency as described above.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the “melting temperature” of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

The term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “low” stringency are often required with nucleic acids that are derived from organisms that are genetically diverse, as the frequency of complementary sequences is usually less.

“Low stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharmacia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1× SSPE, 1.0% SDS at 42° C. when a probe of about 500 nucleotides in length is employed.

It is well known that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q replicase, MDV-1 RNA is the specific template for the replicase (Kacian et al., Proc. Natl. Acad. Sci. USA, 69:3038 (1972), herein incorporated by reference). Other nucleic acid will not be replicated by this amplification enzyme. Similarly, in the case of T7 RNA polymerase, this amplification enzyme has a stringent specificity for its own promoters (Chamberlin et al., Nature, 228:227 (1970), herein incorporated by reference). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics, 4:560 (1989), herein incorporated by reference). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989), herein incorporated by reference).

The term “amplifiable nucleic acid” refers to nucleic acids that may be amplified by any amplification method. It is contemplated that “amplifiable nucleic acid” will usually comprise “sample template.”

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” when used in reference to the polymerase chain reaction refers to the region of nucleic acid bounded by the primers used for polymerase chain reaction. Thus, the “target” is sought to be sorted out from other nucleic acid sequences. A “segment” is defined as a region of nucleic acid within the target sequence.

The term “polymerase chain reaction” (“PCR”) refers to the method of K. B. Mullis U.S. Pat. Nos. 4,683,195, 4,683,202, and 4,965,188, all of which are herein incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

The term “reverse-transcriptase” or “RT-PCR” refers to a type of PCR where the starting material is mRNA. The starting mRNA is enzymatically converted to complementary DNA or “cDNA” using a reverse transcriptase enzyme. The cDNA is then used as a “template” for a “PCR” reaction.

The term “expression” when used in reference to a nucleic acid sequence, such as a gene, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (as when a gene encodes a protein), through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, (1987), herein incorporated by reference). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Maniatis, et al., supra (1987), herein incorporated by reference).

The terms “promoter element,” “promoter,” or “promoter sequence” refer to a DNA sequence that is located at the 5′ end (i.e. precedes) of the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

The term “regulatory region” refers to a gene's 5′ transcribed but untranslated regions, located immediately downstream from the promoter and ending just prior to the translational start of the gene.

The term “promoter region” refers to the region immediately upstream of the coding region of a DNA polymer, and is typically between about 500 bp and 4 kb in length, and is preferably about 1 to 1.5 kb in length.

Promoters may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue (e.g., leaves). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (e.g., peroxidase conjugated) secondary antibody that is specific for the primary antibody is allowed to bind to the sectioned tissue and specific binding detected (e.g., with avidin/biotin) by microscopy.

Promoters may be “constitutive” or “inducible.” The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. Exemplary constitutive plant promoters include, but are not limited to SD Cauliflower Mosaic Virus (CaMV SD; see e.g., U.S. Pat. No. 5,352,605, incorporated herein by reference), mannopine synthase, octopine synthase (ocs), superpromoter (see e.g., WO 95/14098, herein incorporated by reference), and ubi3 promoters (see e.g., Garbarino and Belknap, Plant Mol. Biol. 24:119-127 (1994), herein incorporated by reference). Such promoters have been used successfully to direct the expression of heterologous nucleic acid sequences in transformed plant tissue.

In contrast, an “inducible” promoter is one that is capable of directing a level of transcription of an operably linked nucleic acid sequence in the presence of a stimulus (e.g., heat shock, chemicals, light, etc.) that is different from the level of transcription of the operably linked nucleic acid sequence in the absence of the stimulus.

The term “regulatory element” refers to a genetic element that controls some aspect of the expression of nucleic acid sequence(s). For example, a promoter is a regulatory element that facilitates the initiation of transcription of an operably linked coding region. Other regulatory elements are splicing signals, polyadenylation signals, termination signals, etc.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species).

The term “naturally linked” or “naturally located” when used in reference to the relative positions of nucleic acid sequences means that the nucleic acid sequences exist in nature in the relative positions.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al., Molecular Cloning: A Laboratory Manual, 2nd ed., Cold Spring Harbor Laboratory Press, New York (1989) pp. 16.7-16.8, herein incorporated by reference). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamHI/BclI restriction fragment and directs both termination and polyadenylation (Sambrook, supra, at 16.6-16.7).

The term “vector” refers to nucleic acid molecules that transfer DNA segment(s). Transfer can be into a cell, cell to cell, etc. The term “vehicle” is sometimes used interchangeably with “vector.”

The term “transfection” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (i.e., particle bombardment) and the like.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb in Virol., 52:456 (1973), herein incorporated by reference, has been modified by several groups to optimize conditions for particular types of cells. The art is well aware of these numerous modifications.

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, herein incorporated by reference), and are commercially available (e.g., the helium gas-driven microprojectile accelerator (PDS-1000/He, BioRad).

The term “microwounding” when made in reference to plant tissue refers to the introduction of microscopic wounds in that tissue. Microwounding may be achieved by, for example, particle bombardment as described herein.

The term “transgene” refers to a foreign gene that is placed into an organism by the process of transfection. The term “foreign gene” refers to any nucleic acid (e.g., gene sequence) that is introduced into the genome of an organism by experimental manipulations and may include gene sequences found in that organism so long as the introduced gene does not reside in the same location as does the naturally-occurring gene.

The terms “transformants” or “transformed cells” include the primary transformed cell and cultures derived from that cell without regard to the number of transfers. Resulting progeny may not be precisely identical in DNA content, due to deliberate or inadvertent mutations. Mutant progeny that have the same functionality as screened for in the originally transformed cell are included in the definition of transformants.

The term “selectable marker” refers to a gene which encodes an enzyme having an activity that confers resistance to an antibiotic or drug upon the cell in which the selectable marker is expressed, or which confers expression of a trait which can be detected (e.g., luminescence or fluorescence). Selectable markers may be “positive” or “negative.” Examples of positive selectable markers include the neomycin phosphotrasferase (NPTII) gene that confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “reporter gene” refers to a gene encoding a protein that may be assayed. Examples of reporter genes include, but are not limited to, luciferase (See, e.g., deWet et al., Mol. Cell. Biol. 7:725 (1987) and U.S. Pat Nos. 6,074,859; 5,976,796; 5,674,713; and 5,618,682; all of which are herein incorporated by reference), green fluorescent protein (e.g., GenBank Accession Number U43284; a number of GFP variants are commercially available from CLONTECH Laboratories, Palo Alto, Calif., herein incorporated by reference), chloramphenicol acetyltransferase, β-galactosidase, alkaline phosphatase, and horse radish peroxidase.

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of an siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

The term “posttranscriptional gene silencing” or “PTGS” refers to silencing of gene expression in plants after transcription, and appears to involve the specific degradation of mRNAs synthesized from gene repeats.

The term “cosuppression” refers to silencing of endogenous genes by heterologous genes that share sequence identity with endogenous genes. The term “overexpression” generally refers to the production of a gene product in transgenic organisms that exceeds levels of production in normal or non-transformed organisms. The term “cosuppression” refers to the expression of a foreign gene that has substantial homology to an endogenous gene resulting in the suppression of expression of both the foreign and the endogenous gene. As used herein, the term “altered levels” refers to the production of gene product(s) in transgenic organisms in amounts or proportions that differ from that of normal or non-transformed organisms.

The terms “overexpression” and “overexpressing” and grammatical equivalents, are specifically used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28 S rRNA, an abundant RNA transcript present at essentially the same amount in all tissues, present in each sample can be used as a means of normalizing or standardizing the RAD50 mRNA-specific signal observed on Northern blots).

The terms “Southern blot analysis” and “Southern blot” and “Southern” refer to the analysis of DNA on agarose or acrylamide gels in which DNA is separated or fragmented according to size followed by transfer of the DNA from the gel to a solid support, such as nitrocellulose or a nylon membrane. The immobilized DNA is then exposed to a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al. (1989) Molecular Cloning: A Laboratory Manual, Cold Spring Harbor Press, NY, pp 9.31-9.58, herein incorporated by reference).

The term “Northern blot analysis” and “Northern blot” and “Northern” refer to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al. supra, pp 7.39-7.52, (1989), herein incorporated by reference).

The terms “Western blot analysis” and “Western blot” and “Western” refers to the analysis of protein(s) (or polypeptides) immobilized onto a support such as nitrocellulose or a membrane. A mixture comprising at least one protein is first separated on an acrylamide gel, and the separated proteins are then transferred from the gel to a solid support, such as nitrocellulose or a nylon membrane. The immobilized proteins are exposed to at least one antibody with reactivity against at least one antigen of interest. The bound antibodies may be detected by various methods, including the use of radiolabeled antibodies.

The term “antigenic determinant” refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “isolated” when used in relation to a nucleic acid or polypeptide, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

The term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. As used herein, the term “purified” or “to purify” also refer to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to a plant cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

### DESCRIPTION OF THE INVENTION

The present invention relates to genes, proteins and methods comprising carotenoid monooxygenases in the cytochrome P450 family. In a preferred embodiment, the present invention relates to altering carotenoid ratios in plants and microorganisms using LUT1 ε-hydroxylases and/or CYP97A β-hydroxylases. Thus, the presently claimed invention provides compositions comprising LUT1 genes and coding sequences, and LUT1 polypeptides, and in particular to expression vectors encoding LUT1, CYP97A, CYP97B, and related genes in the CYP97 family and their encoded polypeptides.

The present invention also provides methods for using LUT1 genes, and LUT1 polypeptides; such methods include but are not limited to use of these genes to produce transgenic plants, to produce lutein, to increase lutein, to decrease lutein, to alter carotenoid ratios, to alter phenotypes, and for controlled carotenoid production. It is not meant to limit the present invention to alterations in lutein. In some embodiments, LUT1 alters production of one or more of the following carotenoids, violaxanthin, antheraxanthin, zeaxanthin, neoxanthin, zeinoxanthin, and β-carotenes. In some embodiments, LUT1 polypeptides are overexpressed in transgenic plants, transgenic tissue, transgenic leaves, transgenic seeds, transgenic host cells. It may be desirable to integrate the nucleic acid sequence of interest to the plant genome. Introduction of the nucleic acid sequence of interest into the plant cell genome may be achieved by, for example, heterologous recombination using Agrobacterium-derived sequences.

The present invention also provides methods for using CYP97A genes, and CYP97A polypeptides; such methods include but are not limited to use of these genes to produce transgenic plants, to produce zeaxanthin, to increase zeaxanthin, to decrease zeaxanthin, to alter carotenoid ratios, to alter phenotypes, and for controlled carotenoid production. It is not meant to limit the present invention to alterations in zeaxanthin. In some embodiments, CYP97A alters production of one or more of the following carotenoids, violaxanthin, neoxanthin, lutein, and β-carotenes. In some embodiments, CYP97A polypeptides are overexpressed in transgenic plants, transgenic tissue, transgenic leaves, transgenic seeds, transgenic host cells. It may be desirable to integrate the nucleic acid sequence of interest to the plant genome. Introduction of the nucleic acid sequence of interest into the plant cell genome may be achieved by, for example, by heterologous recombination using Agrobacterium-derived sequences.

The present invention also provides methods for using a combination of CYP97 with non-heme di-iron β-hydroxylase genes and CYP97 with a non-heme di-iron β-hydroxylase polypeptides; such methods include but are not limited to use of these genes to produce transgenic plants, to produce zeaxanthin, to increase zeaxanthin, to decrease zeaxanthin, to alter carotenoid ratios, to alter phenotypes, and for controlled carotenoid production. It is not meant to limit the present invention to alterations in lutein. In some embodiments, a CYP97 with a non-heme di-iron β-hydroxylase alters production of one or more of the following carotenoids, violaxanthin, neoxanthin, lutein, and β-carotenes. In some embodiments, CYP97B polypeptides are overexpressed in transgenic plants, transgenic tissue, transgenic leaves, transgenic seeds, transgenic host cells. It may be desirable to integrate the nucleic acid sequence of interest to the plant genome. Introduction of the nucleic acid sequence of interest into the plant cell genome may be achieved by, for example, by heterologous recombination using Agrobacterium-derived sequences.

The present invention also provides methods for inhibiting LUT1 genes and CYP97A genes, and LUT1 and CYP97A polypeptides; such methods include but are not limited to use of these genes in antisense contructs to produce transgenic plants, to decrease lutein, to decrease zeaxanthin, to increase alpha and beta carotene in different tissues, to alter carotenoid ratios, to alter phenotypes, and for controlled carotenoid production. It is not meant to limit the present invention to particular carotenoids. In some embodiments alterations occur in violaxanthin, antheraxanthin, zeaxanthin, neoxanthin, zeinoxanthin, and β-carotenes. It may be desirable to integrate the nucleic acid sequence of interest to the plant genome. Introduction of the nucleic acid sequence of interest into the plant cell genome may be achieved by, for example, heterologous recombination using Agrobacterium-derived sequences.

The present invention also provides methods for inhibiting LUT1 and CYP97A genes, and LUT1 and CYP97A polypeptides; such methods include but are not limited to use of these genes in antisence contructs to produce transgenic plants, to decrease lutein, to decrease zeaxanthin, to increase alpha and beta carotene in plant tissues, to increase alpha and beta carotene in specific plant tissues, to alter carotenoid ratios, to alter phenotypes, and for controlled carotenoid production. In some embodiments, LUT1 and CYP97A polypeptides are underexpressed in transgenic plants, transgenic tissue, transgenic leaves, transgenic seeds, transgenic host cells. Introduction of the nucleic acid sequence of interest into the plant cell genome may be achieved by, for example, heterologous recombination using Agrobacterium-derived sequences.

The present invention also provides methods for using CYP97B genes, and CYP97B polypeptides; such methods include but are not limited to use of these genes to produce transgenic plants, to alter carotenoid ratios, to alter phenotypes, and for controlled carotenoid production. It may be desirable to target the nucleic acid sequence of interest to a particular locus on the plant genome. In some embodiments, CYP97B polypeptides are overexpressed in transgenic plants, transgenic tissue, transgenic leaves, transgenic seeds, transgenic host cells. In some embodiments, CYP97B polypeptides are underexpressed in transgenic plants, transgenic tissue, transgenic leaves, transgenic seeds, transgenic host cells. Introduction of the nucleic acid sequence of interest into the plant cell genome may be achieved by, for example, heterologous recombination using Agrobacterium-derived sequences.

The present invention is not limited to any particular mechanism of action. Indeed, an understanding of the mechanism of action is not needed to practice the present invention. The following description describes pathways involved in regulating carotenoids, with an emphasis on controlling lutein production or controlling zeaxanthin production or controlling alpha and beta carotene prediction. Also described are methods for identifying genes involved in lutein production or zeaxanthin production, and of the lut1, lut1 mutants and related CYP97 genes discovered through use of these methods. These lut1 and CYP97 related genes have been identified, cloned, and characterized including determination of functional abilities. Further, using the sequences of the present invention, additional CYP97 genes and amino acid sequences are identified, isolated, and characterized for the methods of the present invention. This description also provides methods of identifying, isolated, characterizing and using these genes and their encoded proteins. In addition, the description provides specific, but not limiting, illustrative examples of embodiments of the present invention.

Thus, the presently claimed invention provides compositions comprising LUT1 genes and coding sequences, and LUT1 polypeptides, and in particular to expression vectors encoding LUT1, CYP97A, CYP97B, and related genes in the CYP97 family and their encoded polypeptides. The present invention provides genes from the CYP97 family as designated in Nelson et al. Pharmacogenetics, 6:1-42 (1996), herein incorporated by reference). The present invention also provides methods for using LUT1 genes, and LUT1 polypeptides; such methods include but are not limited to use of these genes to produce transgenic plants, to produce lutein, to increase lutein, to decrease lutein, to alter carotenoid ratios, to alter phenotypes, and for controlled carotenoid production. It may be desirable to target the nucleic acid sequence of interest to a particular locus on the plant genome. Site-directed integration of the nucleic acid sequence of interest into the plant cell genome may be achieved by, for example, homologous recombination using Agrobacterium-derived sequences.

The present invention is not limited to any particular mechanism of action. Indeed, an understanding of the mechanism of action is not needed to practice the present invention. The following description describes pathways involved in regulating carotenoids, with an emphasis on lutein production or lack thereof. Also described are methods for identifying genes involved in lutein production, and of the lut1 mutants and related CYP97 genes discovered through use of these methods. These lut1 and CYP97 related genes have been identified, cloned, and characterized including determination of functional abilities. Further, using the sequences of the present invention, additional CYP97 genes and amino acid sequences are identified, isolated, and characterized for the methods of the present invention. This description also provides methods of identifying, isolating, characterizing and using these genes and their encoded proteins. In addition, the description provides specific, but not limiting, illustrative examples of embodiments of the present invention.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. The term “a nucleotide comprising at least a portion of a gene” may comprise fragments of the gene or the entire gene. The term “cDNA” refers to a nucleotide copy of the “messenger RNA” or “mRNA” for a gene. In some embodiments, cDNA is derived from the mRNA. In some embodiments, cDNA is derived from genomic sequences. In some embodiments, cDNA is derived from EST sequences. In some embodiments, cDNA is derived from assembling portions of coding regions extracted from a variety of BACs, contigs, Scaffolds and the like.

I. Regulation of Carotenoid Production by Hydroxylases

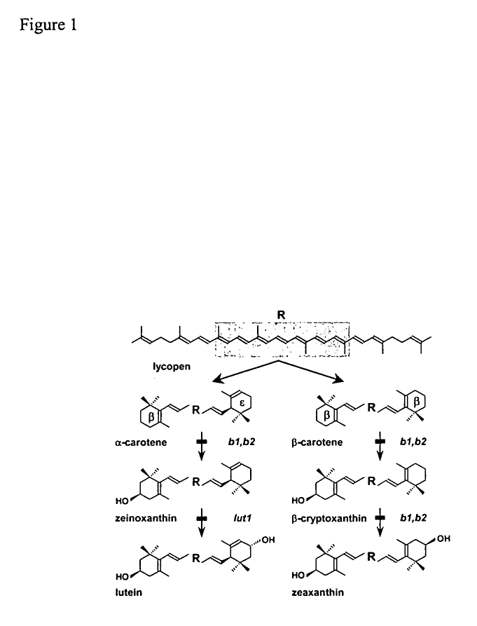

Carotenoids are terpenoid compounds that perform a variety of critical roles in photosystem structure, light harvesting, and photoprotection. Lutein (3R,3′R-β,ε-carotene-3,3′-diol), is the most abundant carotenoid in the majority of plant photosynthetic tissues, where it plays an important role in light harvesting complex II (LHC II) assembly and function. Zeaxanthin (3R,3′R-β,β-carotene-3,3′-diol) is a structural isomer of lutein and is a critical component of non-photochemical quenching (Niyogi, Annu. Rev. Plant Physiol. Plant Mol. Biol. 50, 333-359 (1999); Hirschberg, Curr. Opin. Plant Biol. 4, 210-218 (2001), all of which are herein incorporated by reference). The synthesis of lutein and zeaxanthin involves cyclization of lycopene to form α- and β-carotene, respectively, followed by the introduction of hydroxyl groups onto the ionone rings by a class of enzymes known as carotenoid hydroxylases (FIG. 1).

The term “hydroxylase activity” refers to the ability of a protein to add hydroxyl groups to carbon rings of carotenoids. The terms “having ε-hydroxylase activity” or “ε-ring hydroxylase activity” or “ε-ring hydroxylase” refer to the ability of a protein to hydroxylate an ε-ring. For example an ε-ring hydroxylase converts β,β-carotene into β,ε-carotene-3′-ol (α-carotene with a single hydroxyl group on the ε-ring). The term “having β-hydroxylase activity” or “β-ring hydroxylase activity” or “β-ring hydroxylase” refers to the ability of a protein to hydroxylate a β-ring.

β-Hydroxylases add hydroxyl groups to carbon 3 (C-3) of β-rings while hydroxylation of C-3 on ε-rings is carried out by ε-hydroxylases. Two β-ring hydroxylations of β-carotene yield zeaxanthin while one β- and one ε-ring hydroxylation of α-carotene yield lutein (FIG. 1).

Based on the stereospecific introduction of C-3 hydroxyl groups and the requirement for molecular oxygen, carotenoid hydroxylation reactions were predicted to be catalyzed by mixed function oxygenases, such as the cytochrome P450 enzymes (Walton, et al. Biochem. J. 112, 383-385 (1969); Milborrow, et al. Phytochemistry 21, 2853-2857 (1982); Britton, in Carotenoids: Biosynthesis and Metabolism, Vol. 3, eds. Britton, G., Liaaen-Jensen, S. & Pfander, H. (Basel, Switzerlalnd), pp.13-147 (1998), all of which are herein incorporated by reference). However, β-hydroxylases have been cloned from a variety of photosynthetic and non-photosynthetic bacteria, green algae, and plants (Cunningham and Gantt, Annu. Rev. Plant Physiol. Plant Mol. Biol. 49, 557-583 (1998), herein incorporated by reference) and in three phyla that encode non-heme di-iron proteins that have a fundamentally different hydroxylation reaction mechanism than heme-binding cytochrome P450 enzymes (Shanklin, et al. Biochemistry 33, 12787-12794 (1994), herein incorporated by reference). Biochemical analysis and mutagenesis of pepper (Capsicum annum) β-hydroxylases have confirmed that the enzymes require iron, ferredoxin, and ferredoxin oxido-reductase for activity and that ten of the ten conserved iron-coordinating histidines are required for activity (Bouvier, et al. Biochim. Biophys. Acta. 1391, 320-328 (1998), herein incorporated by reference). The Arabidopsis genome encodes two non-heme di-iron β-hydroxylases (β-hydroxylases 1 and 2) and though both efficiently hydroxylate β-rings, they function poorly with ε-ring containing substrates in vitro (Sun, et al. J. Biol. Chem. 271, 24349-24352 (1996); Tian, et al. Plant Mol. Biol. 47, 379-388 (2001), all of which are herein incorporated by reference).

Early isotope labeling studies have shown that carotenoid hydroxylation reactions are stereospecific (Walton, et al. Biochem. J. 112, 383-385 (1969); Milborrow, et al. Phytochemistry 21, 2853-2857 (1982), all of which are herein incorporated by reference). The chirality of the hydroxylated ε-ring C-3 is opposite to that of the hydroxylated β-ring C-3. This difference in product chirality was an initial suggestion that two distinct hydroxylases are needed for β- and ε-ring hydroxylations and may partially explain why β-hydroxylases function poorly with ε-ring containing substrates in vitro. Mutational studies in Arabidopsis have provided genetic evidence for the existence of a distinct ε-ring ring specific hydroxylase (Pogson, et al. Plant Cell 8, 1627-1639 (1996), herein incorporated by reference). Mutation of the LUT1 locus in Arabidopsis decreased the production of lutein by 80-95% (dependent on plant age) and resulted in accumulation of the monohydroxy precursor zeinoxanthin, a classic phenotype for a mutation affecting a biosynthetic enzyme. ε-Ring hydroxylation was specifically blocked in lut1 and production of β-carotene derived xanthophylls was increased. From these data, it was proposed that LUT1 encodes a function specific for ε-ring hydroxylation (Pogson, et al. Plant Cell 8, 1627-1639 (1996), herein incorporated by reference).

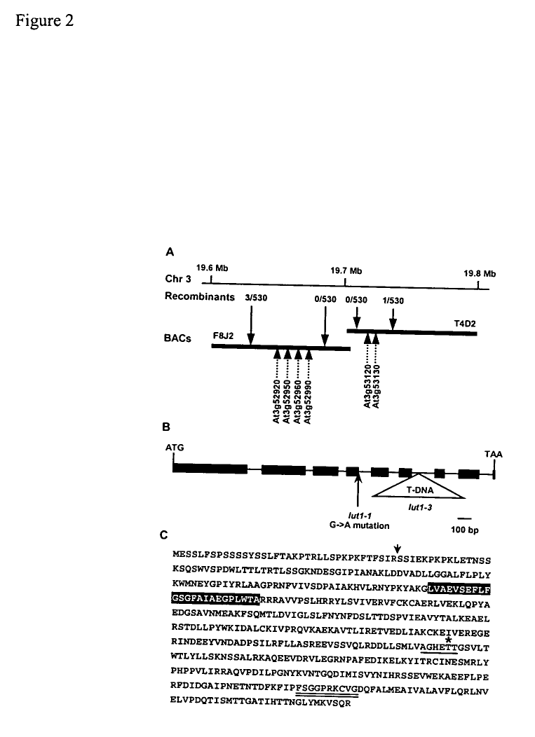

The terms “lut1 gene” or “lut1” or “lutein gene” refer to a plant gene in which a knock-out mutation results in partial or complete loss of lutein, or alteration of carotenoid ratios, in a genetic background where the wild type or non-mutant phenotype (containing the wild type LUT1 gene) produces lutein (as demonstrated in FIGS. 1, 3 and 4). The terms “lut1 gene,” “lut1-1,” “lut1-2” or “lut1-3,”and the like, refer to specific LUT1 alleles e.g., SEQ ID NOs: 6-10 and 23-28. The present invention identifies lut1 genes that are referred to by number, for example, lut1, lut1-1, lut1-2, and lut1-3. The present invention identifies lut1 polypeptides encoded by lut1 genes; these polypeptides are referred to by number, for example, LUT1, lut1-1, lut1-2 and lut1-3, e.g., SEQ ID NOs: 4, 7-9 and 58 and FIGS. 2B and 2C.

The terms “protein,” “polypeptide,” “peptide,” “encoded product,” “amino acid sequence,” are used interchangeably to refer to compounds comprising amino acids joined via peptide bonds and. A “protein” encoded by a gene is not limited to the amino acid sequence encoded by the gene, but includes post-translational modifications of the protein. Where the term “amino acid sequence” is recited herein to refer to an amino acid sequence of a protein molecule, the term “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. The deduced amino acid sequence from a coding nucleic acid sequence includes sequences which are derived from the deduced amino acid sequence and modified by post-translational processing, where modifications include but not limited to glycosylation, hydroxylations, phosphorylations, and amino acid deletions, substitutions, and additions. Thus, an amino acid sequence comprising a deduced amino acid sequence is understood to include post-translational modifications of the encoded and deduced amino acid sequence.

The present invention is not limited to the use of any particular homolog or variant or mutant of LUT 1 protein or lut1 gene. Indeed, in some embodiments a variety of LUT 1 protein or lut1 genes, variants and mutants may be used so long as they retain at least some of the activity of the corresponding wild-type protein. In some embodiments, a variety of LUT 1 protein or lut1 genes, variants and mutants may be used so long as they increase the activity of the corresponding wild-type protein. In particular, it is contemplated that proteins encoded by the nucleic acids of SEQ ID NOs: 5-9, 22-27, 40-48, 53-56, and 58 find use in the present invention. In particular, it is contemplated that nucleic acids encoding proteins that comprise polypeptides at least 40% identical to SEQ ID NO: 1 and the corresponding encoded proteins find use in the present invention. Accordingly in some embodiments, the percent identity is at least 50%, 60%, 70%, 80%, 90%, 95% (or more). In still other embodiments, the nucleic acid sequence further comprises a sequence encoding a cytochrome P450 molecular oxygen binding pocket conserved consensus amino acid motif corresponding to SEQ ID NO: 12. In other embodiments, the nucleic acid sequence further comprises a sequence encoding a conserved transmembrane domain sequence corresponding to SEQ ID NO: 10. In further embodiments, the nucleic acid sequence further comprises a sequence encoding a conserved consensus cysteine motif in P450 molecules corresponding to SEQ ID NO: 14. In other embodiments, the nucleic acid sequence further comprises a sequence encoding a LUT1 conserved consensus cysteine amino acid motif corresponding to SEQ ID NO:15. In still further embodiments, the nucleic acid sequence further comprises a sequence encoding a conserved N-terminal transit peptide for chloroplast-targeting corresponding to SEQ ID NO:11.

Functional variants can be screened for by expressing the variant in an appropriate vector (described in more detail below) in a plant cell and analyzing the carotenoids produced by the plant.

II. Methods for Identifying Genes Involved in Hydroxylation of Carotenoid Molecules

The present invention provides methods for identifying genes involved in carotenoid production. These methods include first screening a mutagenized population of plants (for example, Arabidopsis plants) for recessive mutants that exhibit a constitutive phenotype, or in other words mutants that lack lutein and thus lack the ability to hydroxylate epsilon rings of carotenoid molecules. Prior attempts to clone an ε-ring specific hydroxylase by sequence-based similarity to β-hydroxylases in Arabidopsis were not successful and only identified the β-hydroxylase 2 gene (Tian, et al. Plant Mol. Biol. 47, 379-388 (2001), herein incorporated by reference). A thorough search of the fully sequenced Arabidopsis genome also failed to identify any additional genes bearing significant similarity to β-hydroxylases from plants, cyanobacteria, and non-photosynthetic bacteria (Tian, et al. Plant Mol. Biol. 47, 379-388 (2001), herein incorporated by reference). These results suggested that the ε-hydroxylase defines a structurally distinct carotenoid hydroxylase family. We report here identification of the LUT1 locus by positional cloning and show that LUT1 indeed defines a new class of carotenoid hydroxylases within the cytochrome P450 superfamily.

The LUT1 locus has previously been mapped to the bottom arm of chromosome 3 at 67±3 cM (Tian, et al. Plant Mol. Biol. 47, 379-388 (2001), herein incorporated by reference). For fine mapping of the locus, 530 plants homozygous for the lut1 mutation were identified from approximately 2,000 plants in a segregating F₂ mapping population. Using SSLP markers, LUT1 was initially localized to an interval spanning two BAC clones (F8J2 and T4D2) and was further delineated to a 100 kb interval containing 30 predicted proteins (FIG. 2A). The term “BAC” and “bacterial artificial chromosome” refers to a vector carrying a genomic DNA insert, typically 100-200 kb. The term “SSLP” and “simple sequence length polymorphisms” refers to a unit sequence of DNA (2 to 4 bp) that is repeated multiple times in tandem wherein common examples of these in mammalian genomes include runs of dinucleotide or trinucleotide repeats (for example, CACACACACACACACACA).” As with all other carotenoid biosynthetic enzymes, the LUT1 gene product is predicted to be chloroplast-targeted and within the 100 kb interval containing LUT1, six proteins were predicted as being chloroplast-targeted by the TargetP prediction software (http://www.cbs.dtu.dk/services/TargetP). One of these chloroplast-targeted proteins, At3g53130, is a member of the cytochrome P450 monooxygenase family (CYP97C1). Cytochrome P450 monooxygenases are heme-binding proteins that insert a single oxygen atom into substrates, e.g. hydroxylation reactions, and therefore At3g53130 was considered to be a strong candidate for LUT1.

The terms “CYP97,” “CYP97A,” “CYP97B,” “CYP97C,” “CYP97-like” and “CYP97 family” refer to groups of cytochrome P450 genes and proteins. The terms “CYP97” and “CYP97 family” refers to any and all of “CYP97A,” “CYP97B,” “CYP97C and “CYP97-like” genes and proteins. In some embodiments, cytochrome P450s in a same family share at least 40% identity. In some embodiments, genes in the same subfamily, (e.g. CYP97C), usually share at least 55% identity. However there are a few exceptions, especially in plants, due to frequent gene duplication and shuffling within the genome. In one embodiment, sequence identity among P450s from Arabidopsis can be less than 20%. For the purposes of the present invention, family assignment is based upon a combination of sequence identity, phylogeny and gene organization (Nelson et al. Pharmacogenetics 6:1-42 (1996), herein incorporated by reference).

III. Hydroxylase Genes; Regulators of Lutein Production

The interactions and functional redundancies of the three known carotenoid hydroxylases in Arabidopsis (β-hydroxylases 1 and 2, LUT1) have been studied in vivo by isolating mutations disrupting each gene and generating multiple hydroxylase deficient mutant genotypes (Tian, et al. Plant Cell 15, 1320-1332 (2003), herein incorporated by reference). In the β-hydroxylase 1/β-hydroxylase 2 double null mutant (b1 b2), where both known β-hydroxylases were eliminated, hydroxylated β-ring groups were still synthesized at significant levels (75% of wild type), indicating that an additional β-ring hydroxylation activity exists in vivo. The ethyl methane sulfonate (EMS)-derived lut1-2 mutation was introduced into the b1 b2 background to address whether this additional β-hydroxylase activity might be a secondary function of the ε-hydroxylase or due to a third unrelated β-hydroxylase. Hydroxylated β-ring groups were further reduced to 60% of wild type levels in the lut1-2 b1 b2 triple mutant (Tian, et al. Plant Cell 15, 1320-1332 (2003), herein incorporated by reference) suggesting that LUT1 is capable of some degree of β-ring hydroxylation in vivo. However, a caveat of this experiment is that LUT1 activity may not have been completely eliminated in the EMS-derived lut1-2 mutant and the issue of whether the remaining β-ring hydroxylation in lut1-2 b1 b2 was due to residual LUT1 activity or the presence of a third unrelated β-hydroxylase could not be resolved. Cloning of the LUT1 locus and generation of a null ε-hydroxylase mutant are required to further understanding of in vivo carotenoid hydroxylase activity and for applying molecular genetic approaches to study carotenoid hydroxylase functions in vivo.

IV. Positional Cloning of LUT1

The term “positional cloning” refers to an identification of a gene based on its physical location in the genome. Homozygous lut1-1 (ecotype Columbia) was crossed to wild type Landsberg erecta. F₂ progeny homozygous for the lut1 mutation were identified by a thin-layer chromatography (TLC) screening method. Briefly, carotenoid samples were extracted as described (Tian, et al. Plant Mol. Biol. 47, 379-388 (2001), herein incorporated by reference) resuspended in ethyl acetate, spotted on a silica TLC plate (J. T. Baker, Phillipsburg, N.J.), and developed in 90:10 (v:v) hexane: isopropanol. F₂ plants homozygous for lut1 contain a characteristic extra yellow band due to accumulation of zeinoxanthin.

Genomic DNA from homozygous lut1 F₂ plants was isolated using the DNAzol reagent following the manufacturer's instructions (Invitrogen, Carlsbad, Calif.). PCR reactions were performed with 1 μl of genomic DNA in a 20 μl reaction mixture. The PCR program was 94° C. for 3 min, 60 cycles of 94° C. for 15 s, 50° C.-60° C. (the annealing temperature was optimized for each specific pair of primers) for 30 s, 72° C. for 30 s, and finally 72° C. for 10 min. A portion of the PCR product was then separated on a 3% agarose gel. lut1 had been previously mapped to 67±3 cM on chromosome 3 (Tian, et al. Plant Mol. Biol. 47, 379-388 (2001). Simple Sequence Length Polymorphism (SSLP) markers for fine mapping in this interval were designed based on the insertions/deletions (INDELs) information obtained from the Monsanto website: http://www.arabidopsis.org/Cereon/.

A. Mutant Complementation, Characterization, and the Identification of LUT1

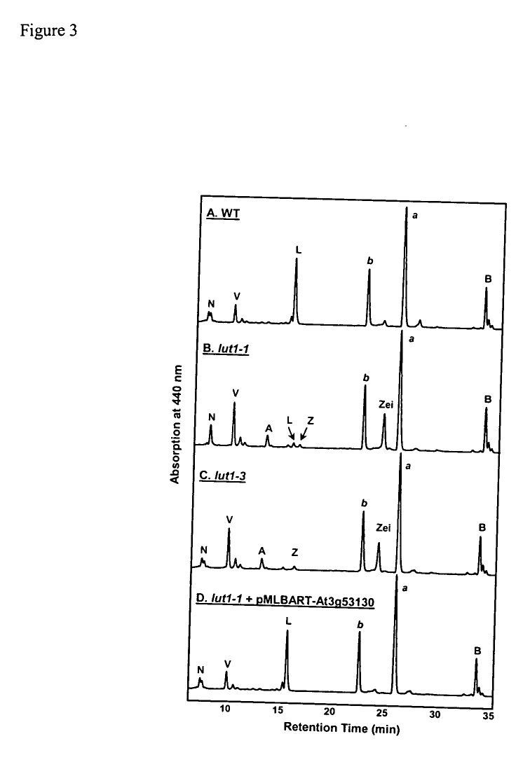

The identity of At3g53130 as containing lut1 was initially demonstrated by molecular complementation analysis. Homozygous lut1-1 mutants were transformed with a 4.2 kb genomic DNA fragment from wild type Columbia (the background of lut1) containing the At3g53130 coding region, 1.0 kb upstream of the start codon, and 0.7 kb downstream of the stop codon. Eight independent transformants were selected and these showed a wild type lutein level when analyzed by HPLC (FIG. 3D). These data indicate that At3g53130 genomic DNA can complement the lut1 mutation.

To determine the molecular basis of the lut1 mutations, both original EMS-derived lut1 alleles (Pogson, et al. Plant Cell 8, 1627-1639 (1996), herein incorporated by reference) were sequenced. The lut1-1 allele contains a G to A mutation at the highly conserved exon/intron splice junction (5′ AG/GT, the mutated G is in bold) that would cause an error in RNA splicing and lead to production of a mistranslated protein (FIG. 2B). The coding region of the lut1-2 allele was fully sequenced but no mutations were identified. However, a rearrangement in the upstream region of the lut1-2 allele was identified by Southern blot analysis but was not characterized further (data not shown). A third lut1 allele, lut1-3, was identified by screening a T-DNA knockout population using At3g53130-specific primers. lut1-3 contains a T-DNA insertion in the sixth intron of the LUT1 gene (FIG. 2B).

In order to compare the impact of different lut1 alleles on carotenoid composition, total carotenoids were extracted from four-week old wild type, lut1-1, lut1-2 (data not shown), and lut1-3 plants and separated by HPLC (FIGS. 3A-C). Lut1-1 and lut1-2 accumulated the monohydroxy biosynthetic intermediate zeinoxanthin and contained 8% of wild type lutein, consistent with prior report (Pogson, et al. Plant Cell 8, 1627-1639 (1996, herein incorporated by reference). In contrast, though lut1-3 also accumulated zeinoxanthin it lacked lutein (FIG. 3C), indicating that ε-ring hydroxylation function is eliminated by disruption of the At3g53130 gene. The lut1-3 phenotype also indicates that redundant ε-ring hydroxylation activities are not present in leaves and that the previously reported EMS-mutagenized lut1-1 and lut1-2 alleles are indeed leaky for ε-ring hydroxylation activity (FIG. 3B; Pogson, et al. Plant Cell 8, 1627-1639 (1996, herein incorporated by reference). Taken together, the complementation of the lut1-1 mutation with a wild type At3g53130 gene, the point mutation at a conserved splice site in the lut1-1 allele, and the phenotype of the At3g53130 T-DNA knockout mutant conclusively demonstrate that At3g53130 includes the LUT1 locus.

B. LUT1 Encodes a Chloroplast-Targeted Cytochrome P450 with a Single Transmembrane Domain

The deduced amino acid sequence of LUT1 contains several features characteristic of cytochrome P450 enzymes (FIG. 2C). Cytochrome P450 monooxygenases contain a consensus sequence of (A/G)GX(D/E)T(T/S) that forms a binding pocket for molecular oxygen with the invariant Thr residue playing a critical role in oxygen binding in both prokaryotic and eukaryotic cytochrome P450s (Chapple, Annu. Rev. Plant Physiol. Plant Mol. Biol. 49, 311-343 (1998, herein incorporated by reference). In the deduced LUT1 protein sequence, this oxygen-binding pocket is highly conserved (single underlined amino acids in FIG. 2C). The conserved sequence around the heme-binding cysteine residue for cytochrome P450 type enzymes is FXXGXXXCXG, and is also present in LUT1 (double underlined amino acids in FIG. 2C).

The chloroplast transit peptide prediction software ChloroP v 1.1 (http://www.cbs.dtu.dk/services/ChloroP/) predicts an N-terminal transit peptide in LUT1 that is cleaved between Arg-36 and Ser-37 (FIG. 2C). The predicted chloroplast localization for LUT1 is consistent with the subcellular localization of carotenoid biosynthesis in higher plants (Cunningham and Gantt, Annu. Rev. Plant Physiol. Plant Mol. Biol. 49, 557-583 (1998) but is uncommon for a plant cytochrome P450. Out of the 272 predicted cytochrome P450s in the Arabidopsis genome, only nine, including LUT1, are predicted to be chloroplast-targeted (Schuler and Werck-Reichhart, Annu. Rev. Plant Biol. 54, 629-667 (2003, herein incorporated by reference). LUT1 also contains a single predicted transmembrane domain (shaded box, FIG. 2C), which contrasts with the four transmembrane domains predicted for the non-heme di-iron β-hydroxylases (Cunningham and Gantt, Annu. Rev. Plant Physiol. Plant Mol. Biol. 49, 557-583 (1998, herein incorporated by reference). Initial attempts to express and assay LUT1 protein in yeast were unsuccessful.

C. LUT1 Gene Expression and in vivo Activity in the β-Hydroxylase Deficient Backgrounds

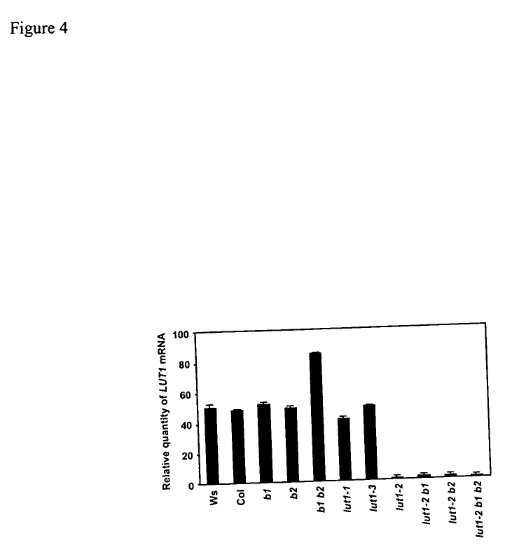

Characterization of previously isolated T-DNA knockouts in the two Arabidopsis β-hydroxylase genes suggested that β- and ε-hydroxylases have overlapping functions in vivo (Tian, et al. Plant Cell 15, 1320-1332 (2003, herein incorporated by reference). In order to investigate whether ε-hydroxylase expression is affected in the various carotenoid hydroxylase mutant backgrounds, steady state LUT1 mRNA levels were quantified by real-time PCR (FIG. 4). The LUT1 TaqMan probe hybridizes 336 bp downstream from the start codon. LUT1 mRNA levels are not significantly different from wild type in the β-hydroxylase single mutants (b1 and b2), but are significantly increased in the β-hydroxylase double mutant b1 b2 (FIG. 4). LUT1 mRNA levels in lut1-2 alone and in combination with various β-hydroxylase mutant loci (i.e. lut1-2 b1, lut1-2 b2, and lut1-2 b1 b2) are similar and reduced to 2% of wild type levels, consistent with the rearrangement of the upstream region in lut1-2 negatively impacting LUT1 transcription. The steady-state levels of modified LUT1 transcript in lut1-1 and lut1-3 are similar to wild type transcript levels suggesting that although LUT1 activity is negatively impacted in each mutant, there is little impact on LUT1 transcription.

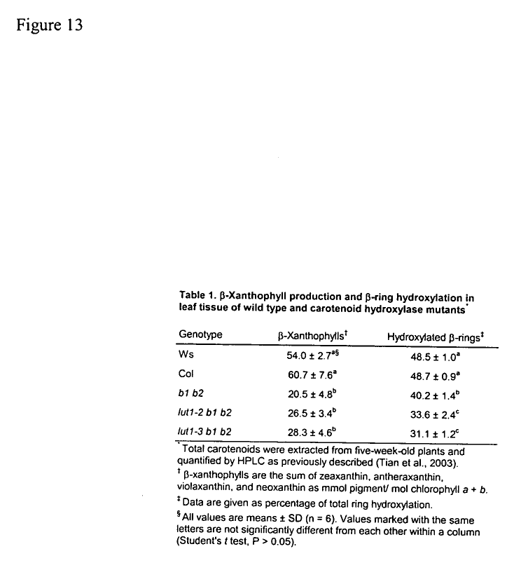

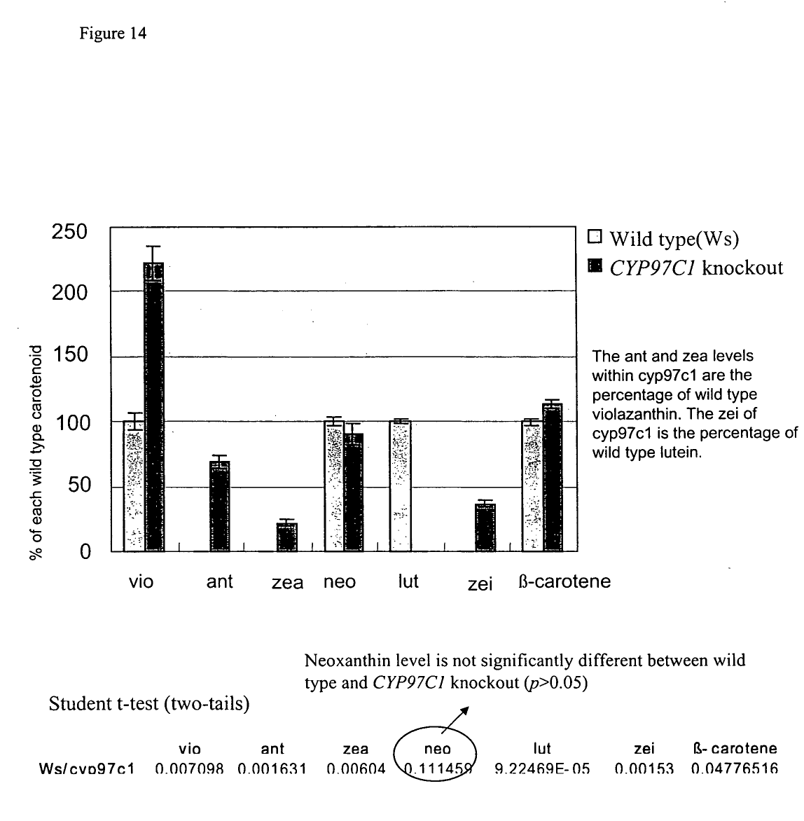

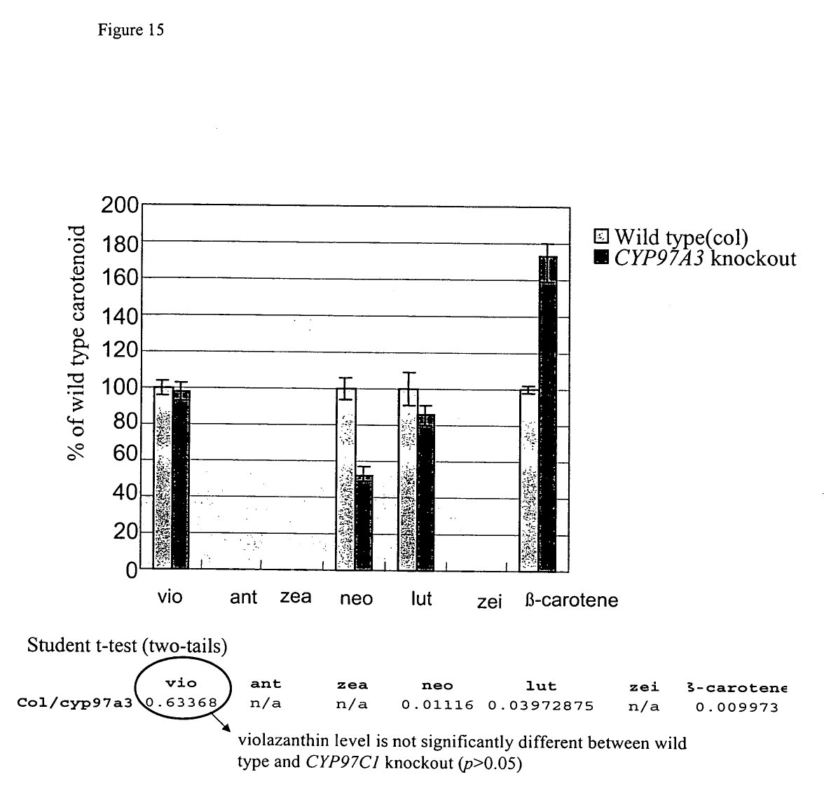

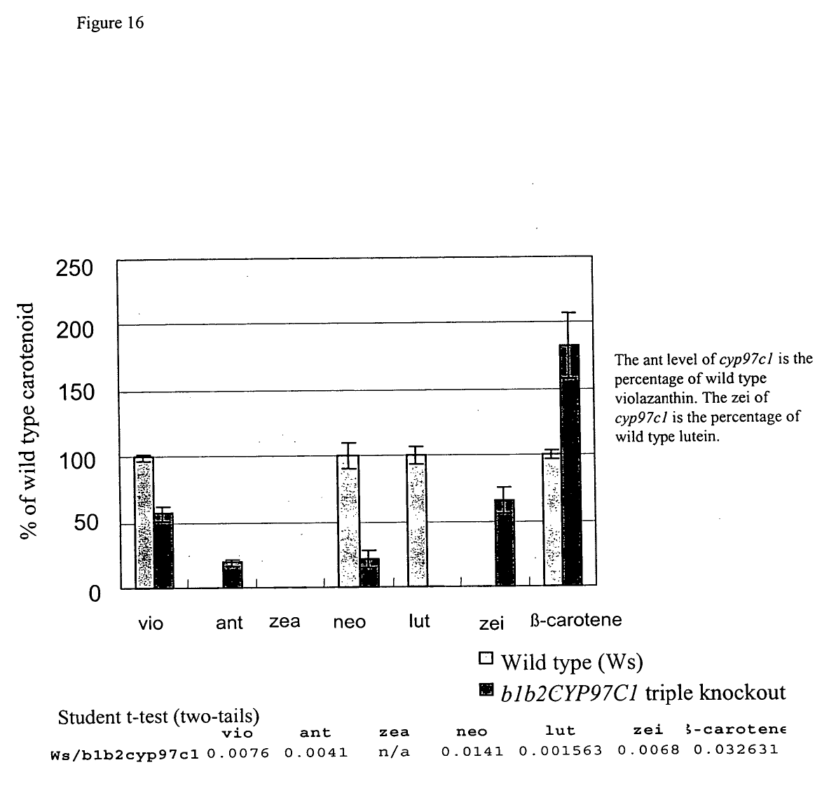

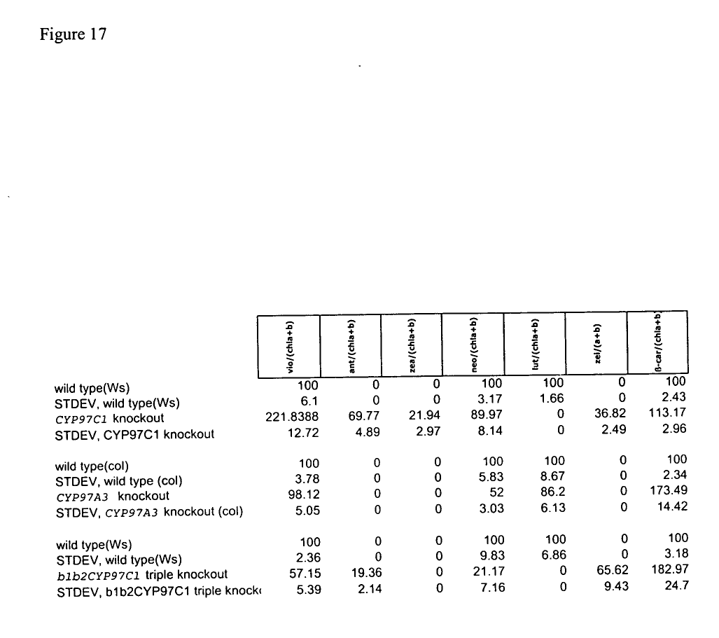

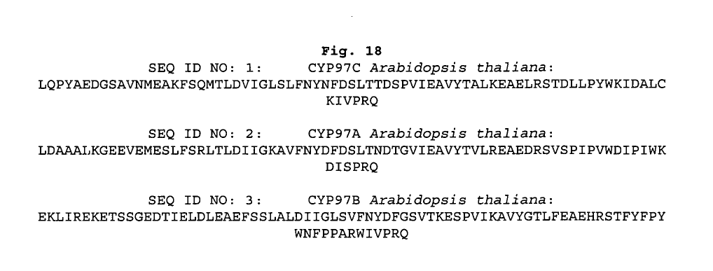

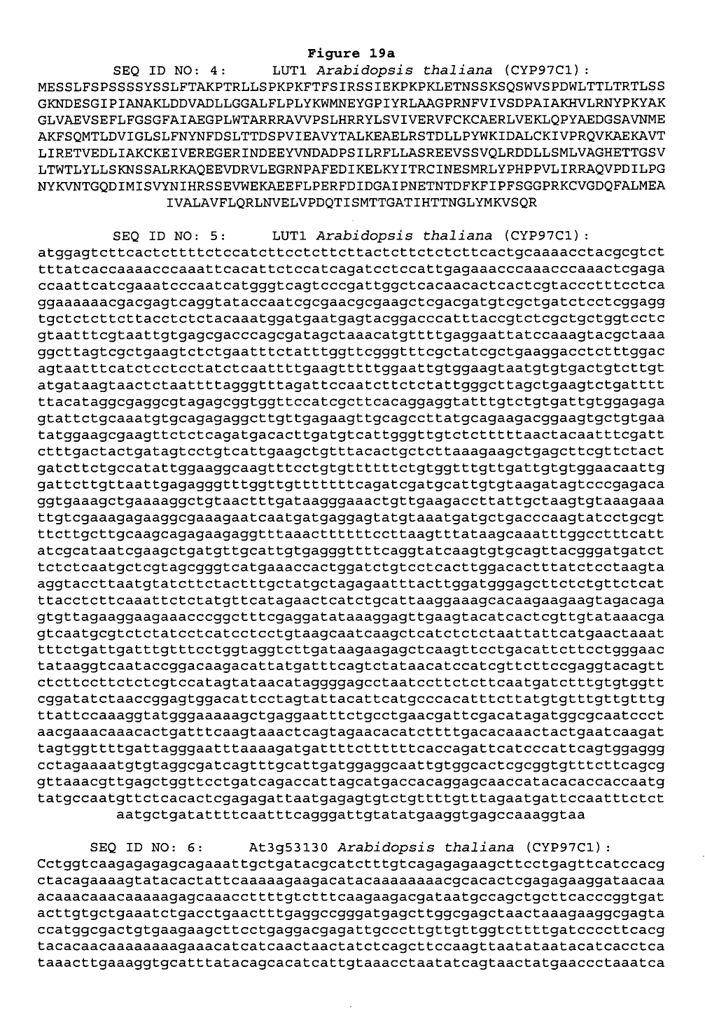

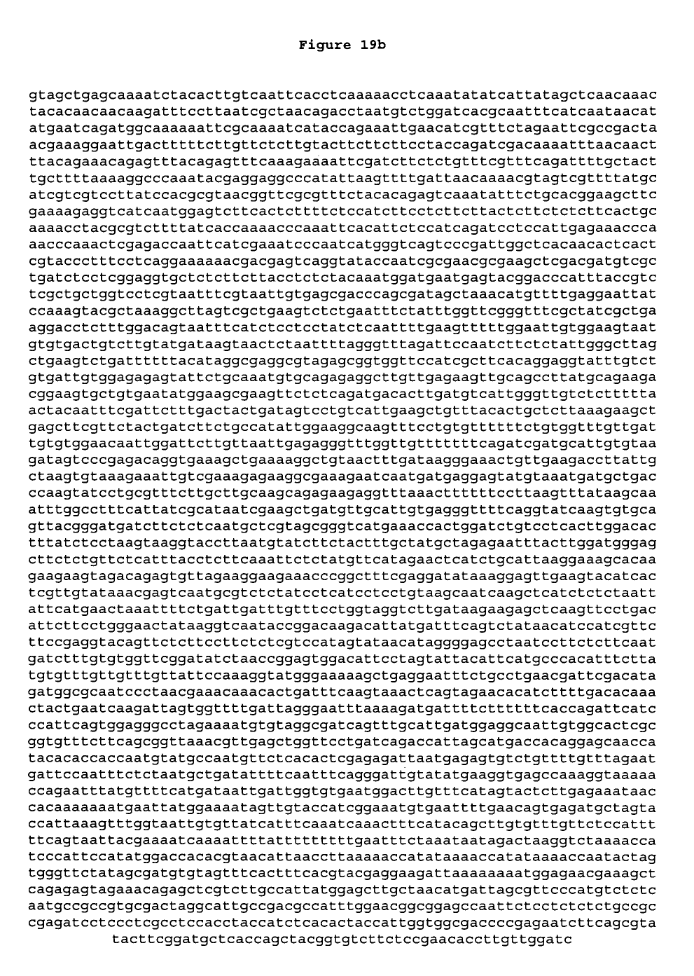

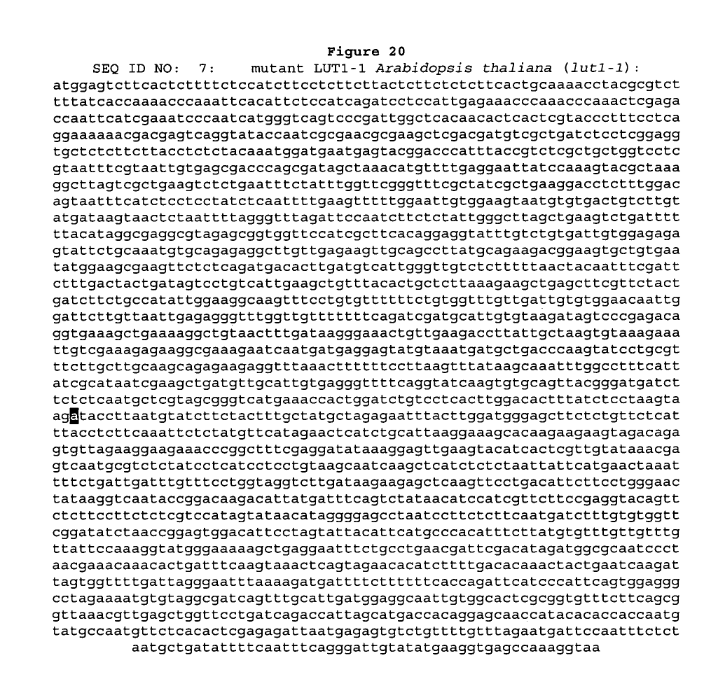

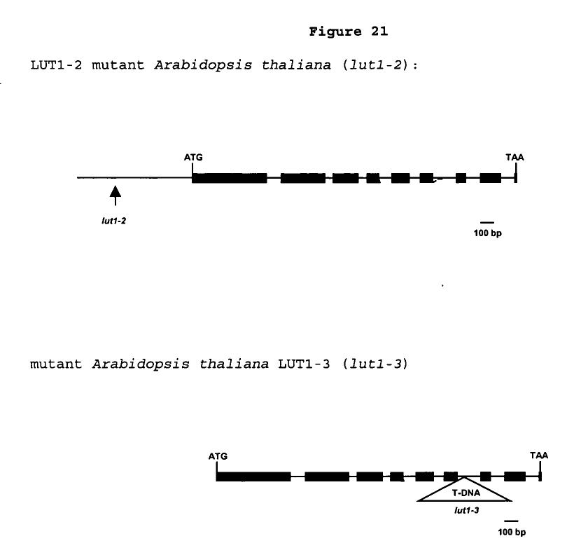

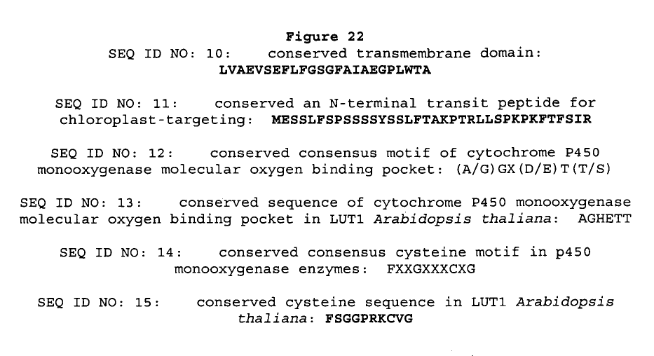

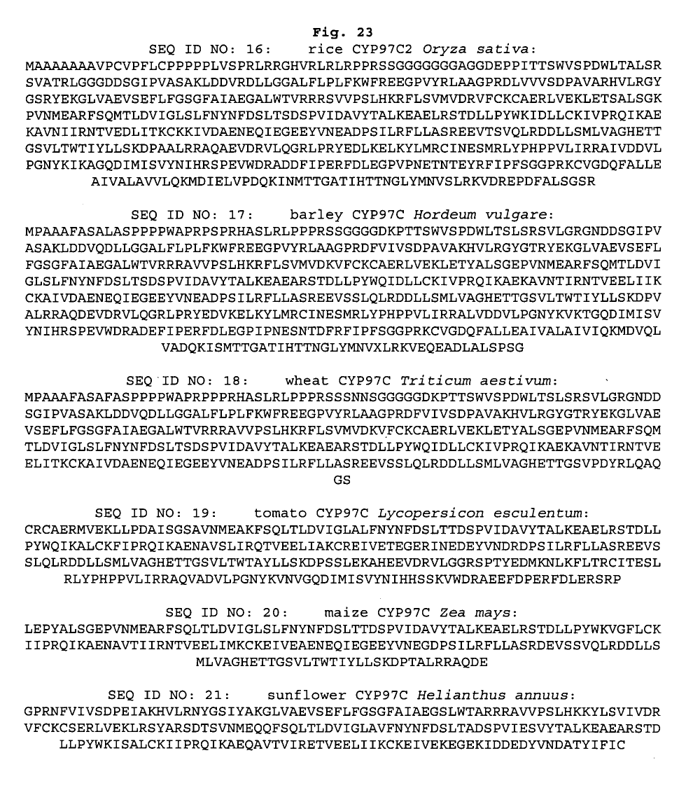

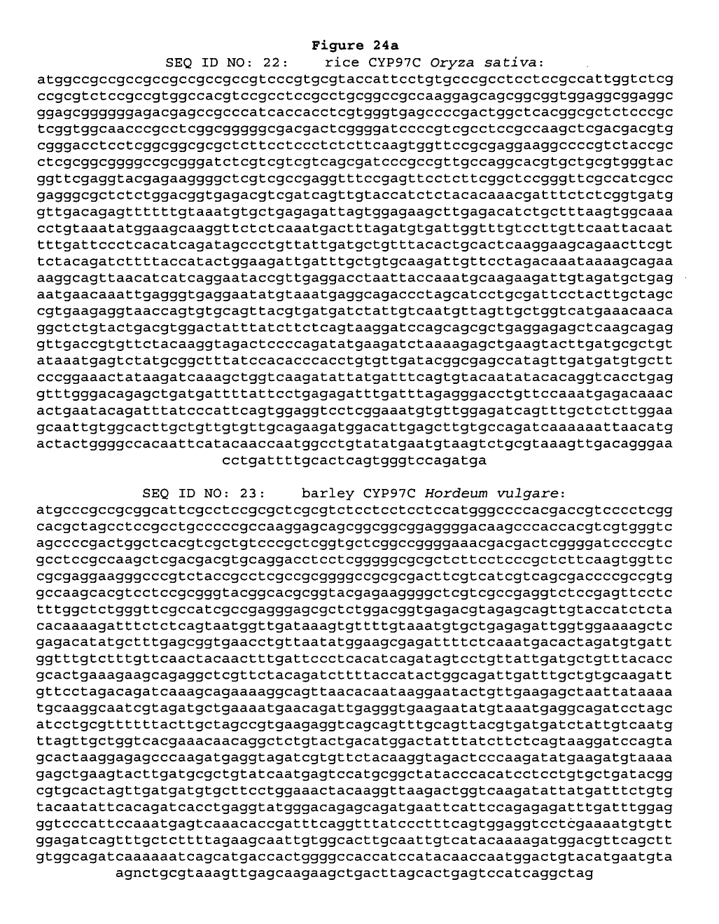

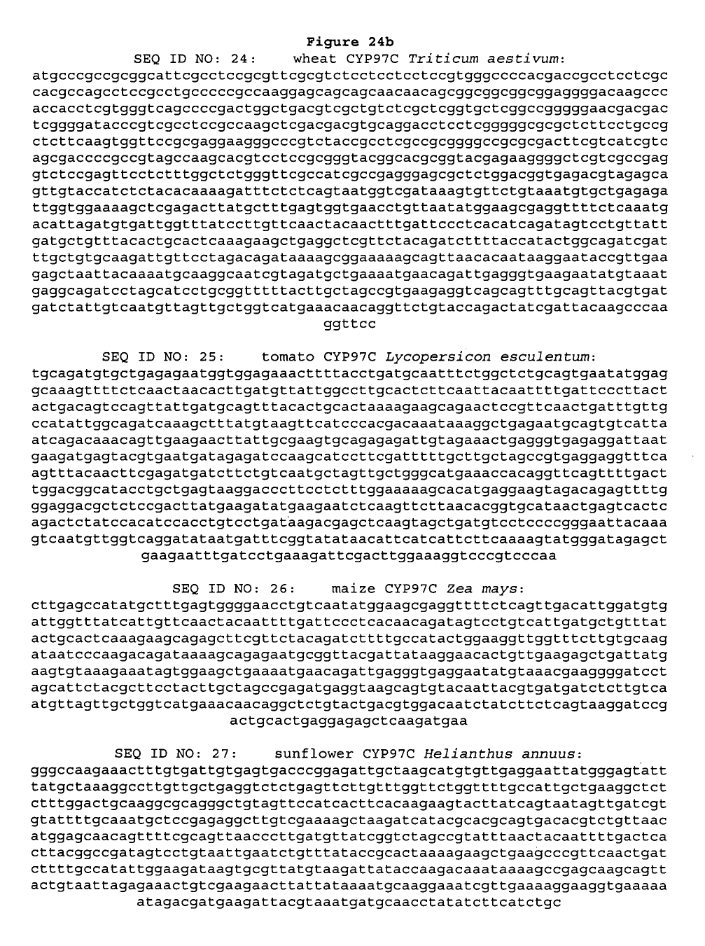

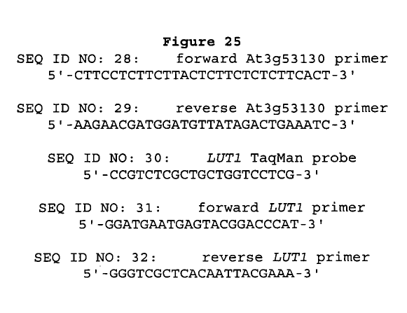

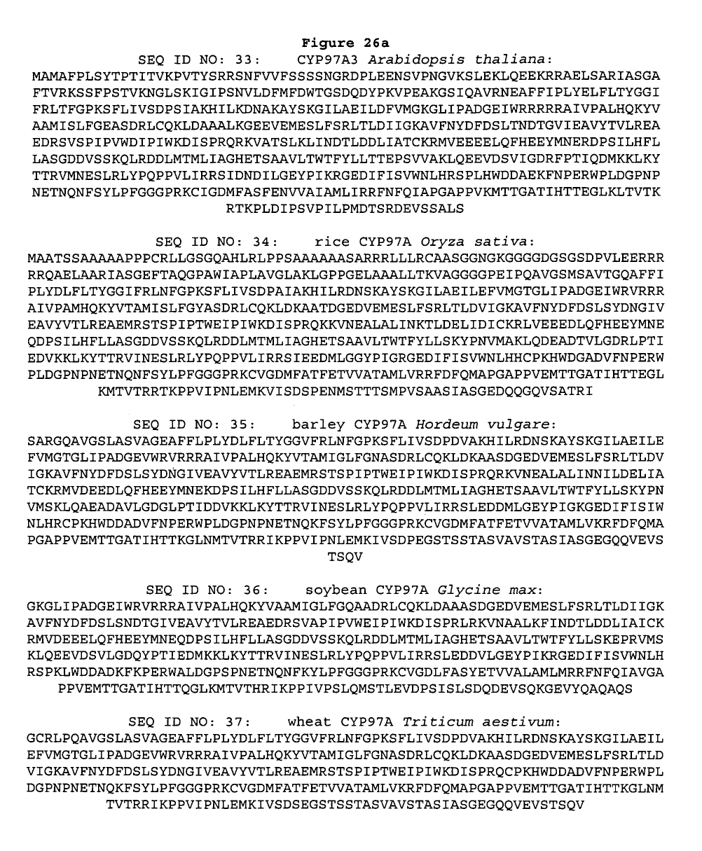

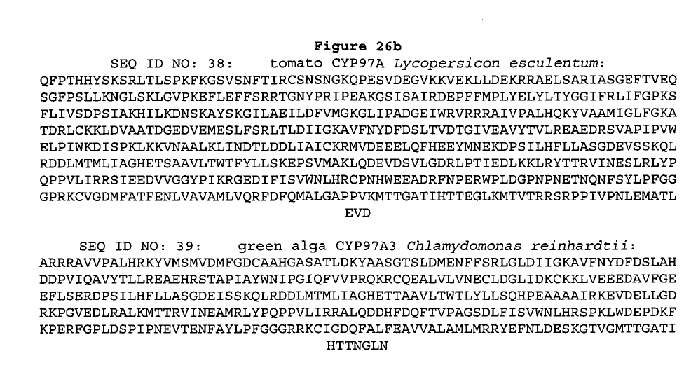

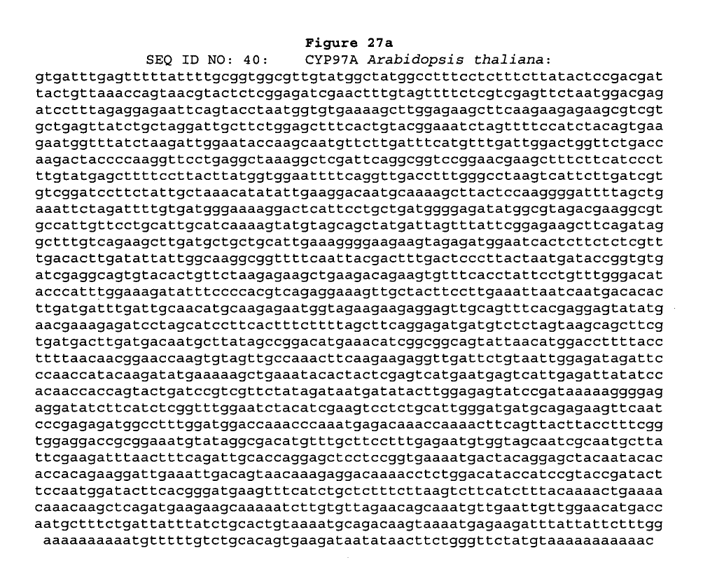

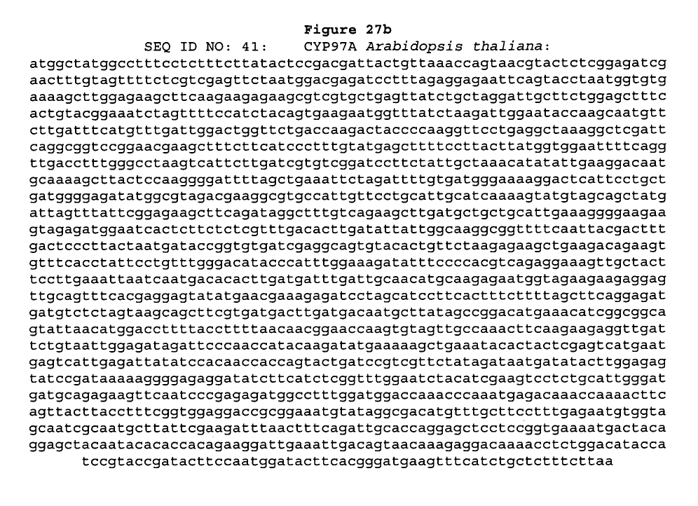

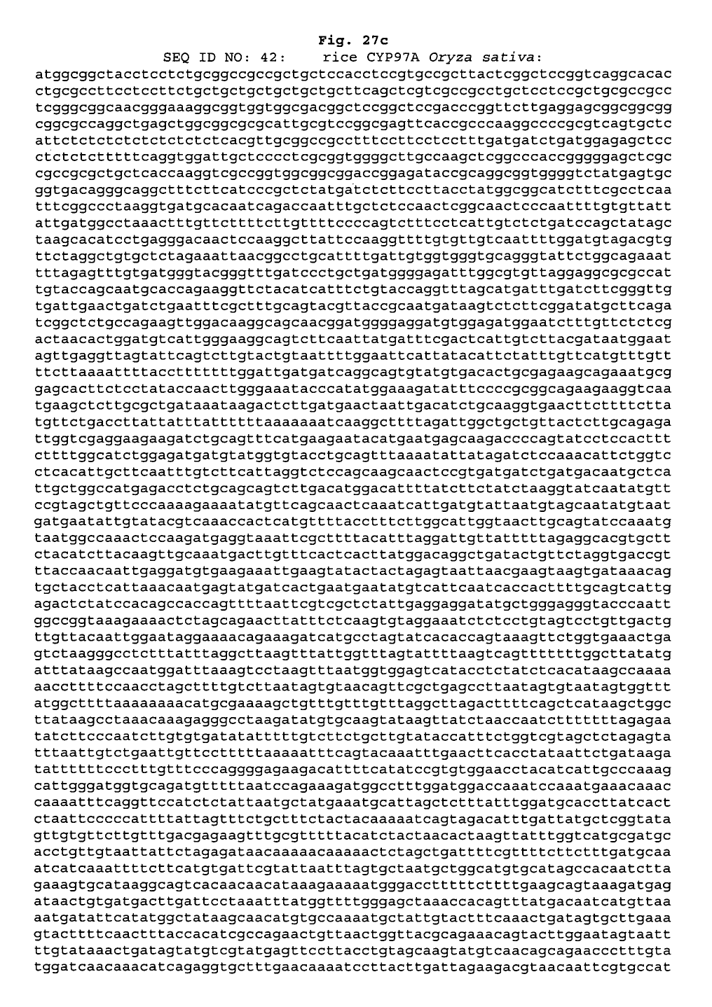

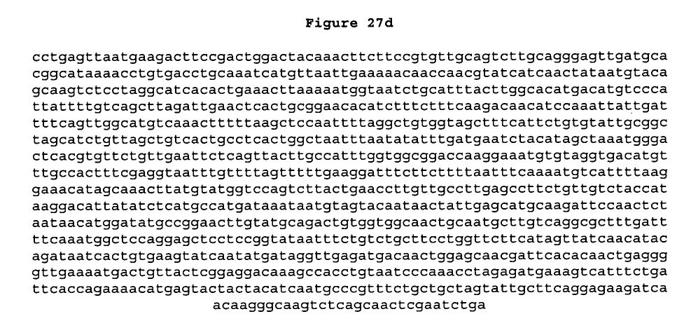

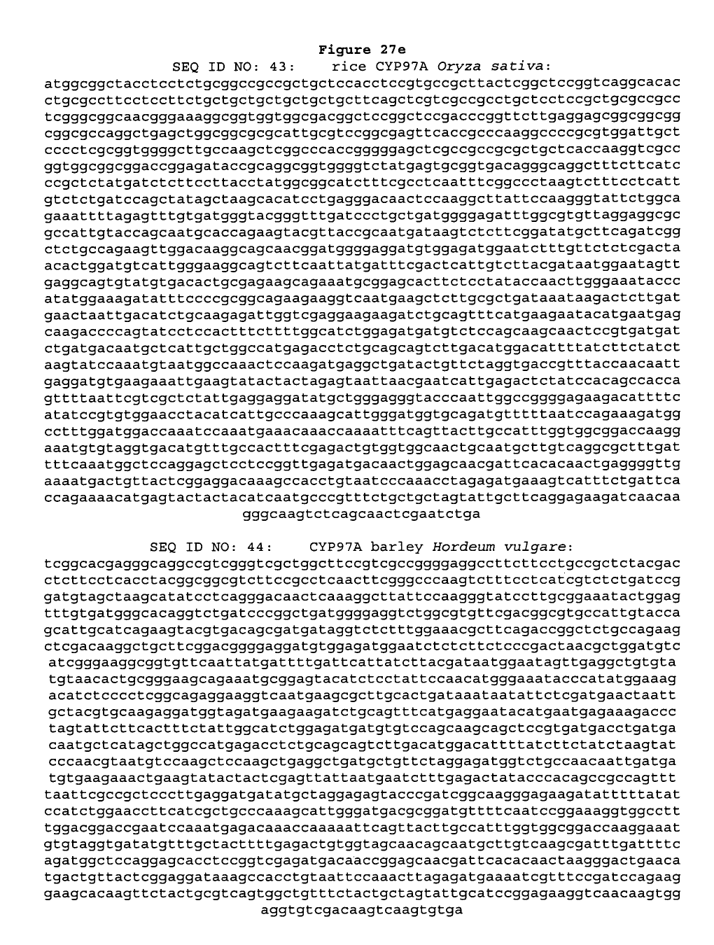

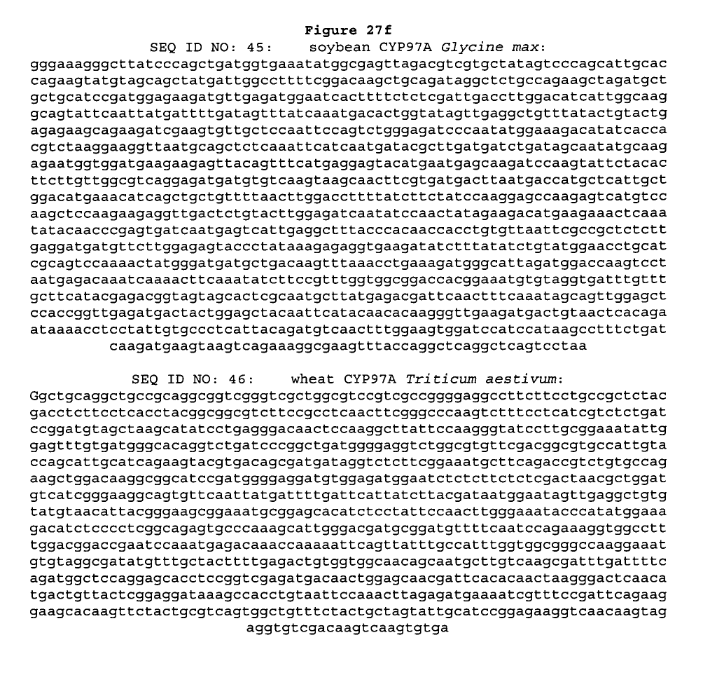

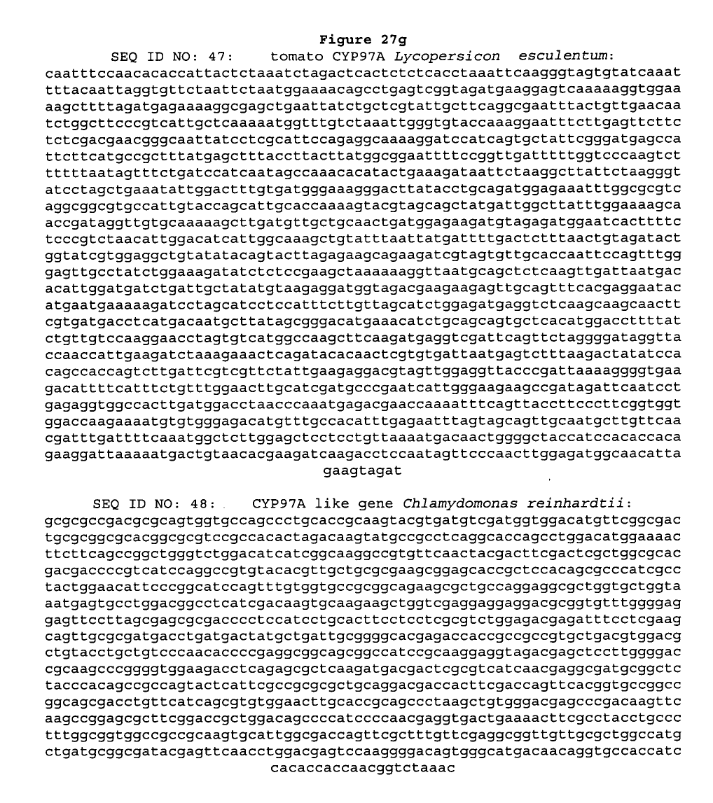

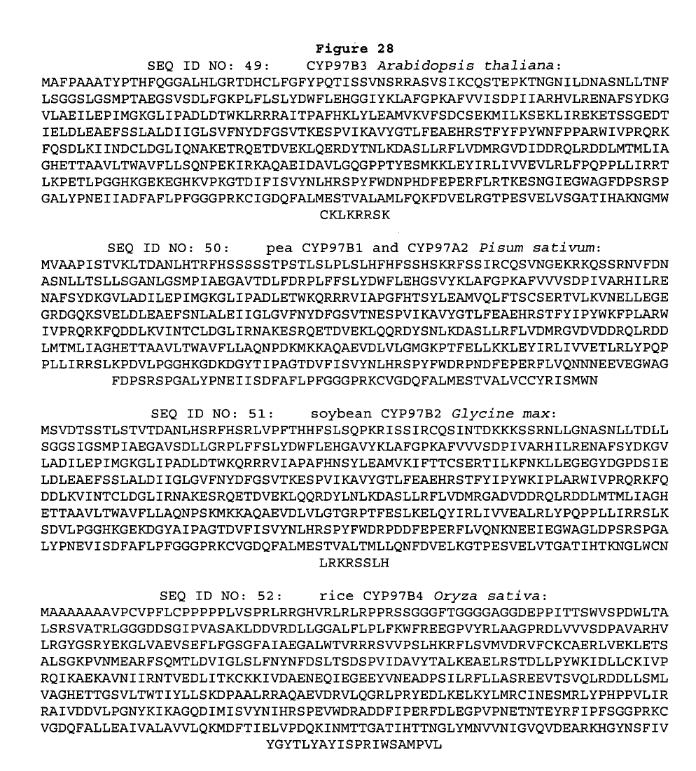

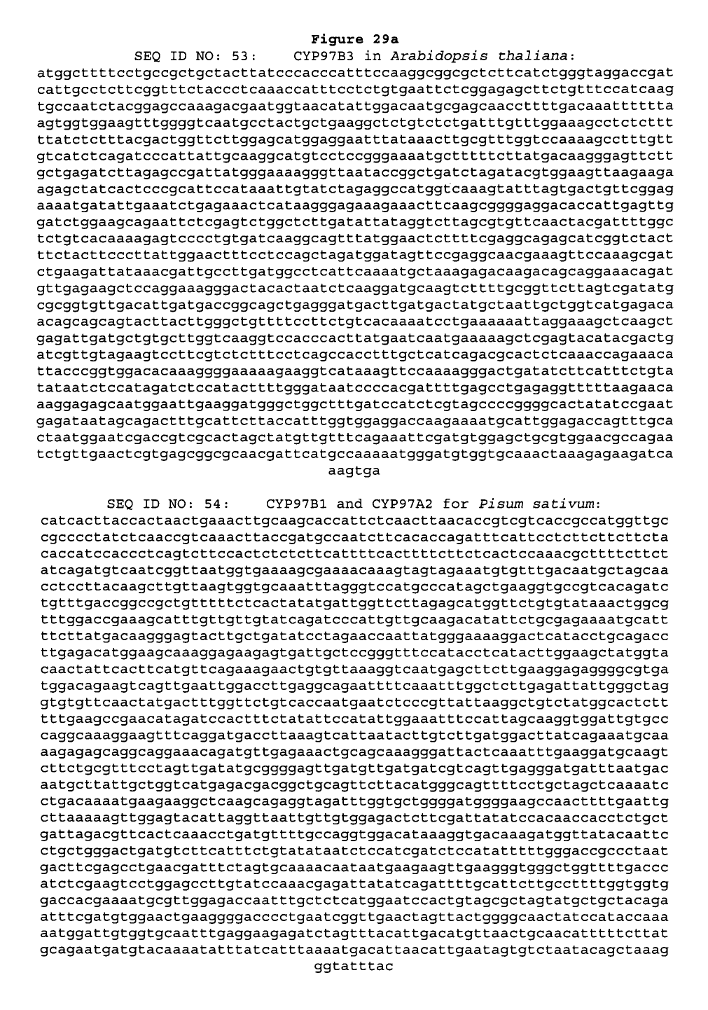

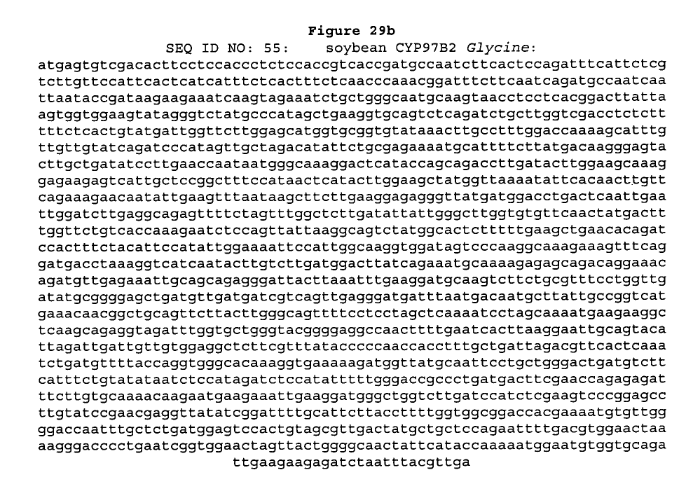

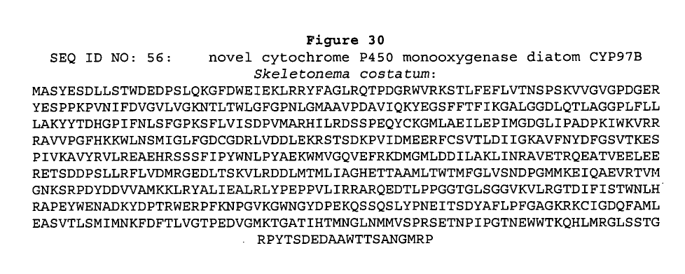

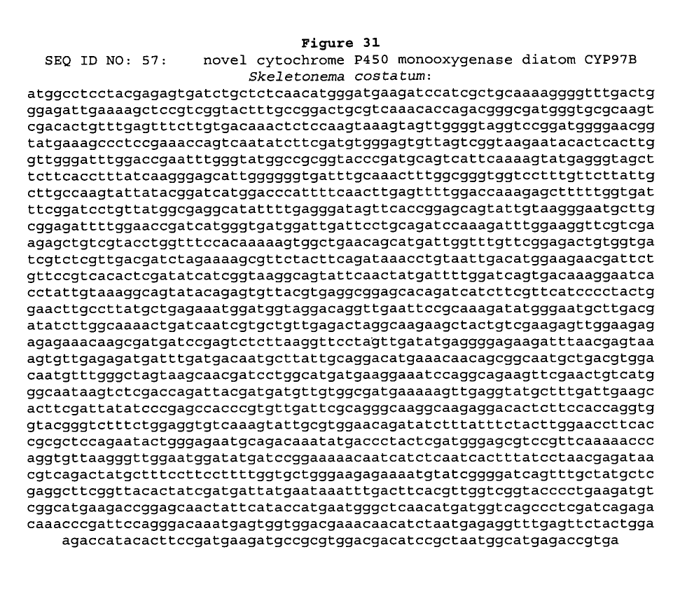

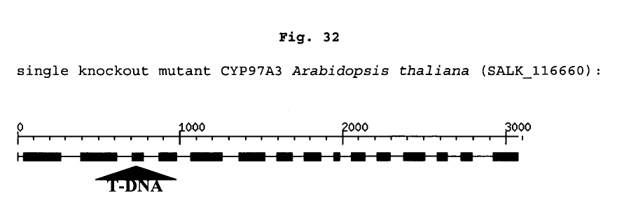

The phenotype of the previously isolated lut1-2 b1 b2 mutant was not conclusive due to the leaky nature of the EMS-derived lut1-2 allele. Cloning of LUT1 and isolation of the LUT1 knockout mutant, lut1-3, allow for the complete elimination of LUT1 activity in vivo. Lut1-3 was crossed to b1 b2 and homozygous lut1-3 b1 b2 mutants were isolated. There was no lutein production in the lut1-3 b1 b2 triple mutant (data not shown), consistent with the lut1-3 single mutant phenotype (FIG. 3C). The total moles of β-carotene derived xanthophylls produced are not significantly different between lut1-2 b1 b2 and lut1-3 b1 b2 (FIG. 13). However, when one considers the total moles of hydroxylated β-rings produced in each mutant (which includes hydroxylated β-ring in zeinoxanthin), total hydroxylated β-rings are significantly reduced in lut1-2 b1 b2 and lut1-3 b1 b2 compared to b1 b2, suggesting that LUT1 also has β-ring hydroxylation activity in vivo (FIG. 13). In addition, the presence of β-carotene derived xanthophylls in the triple knockout mutant lut1-3 b1 b2 indicates a third β-hydroxylase must exist in vivo (FIG. 13).

D. LUT1 (CYP97) Homologs in Other Species

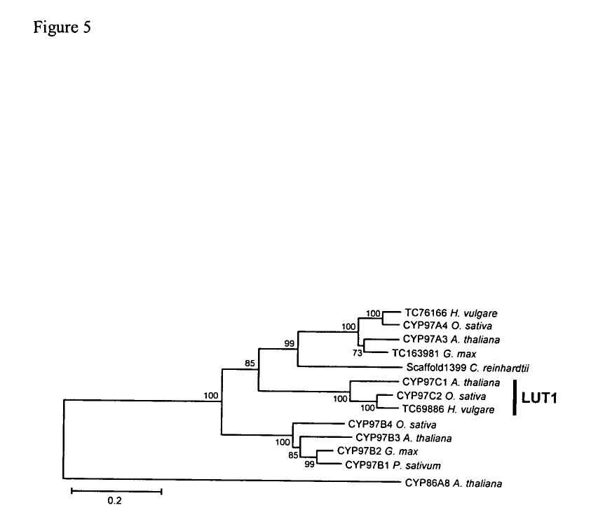

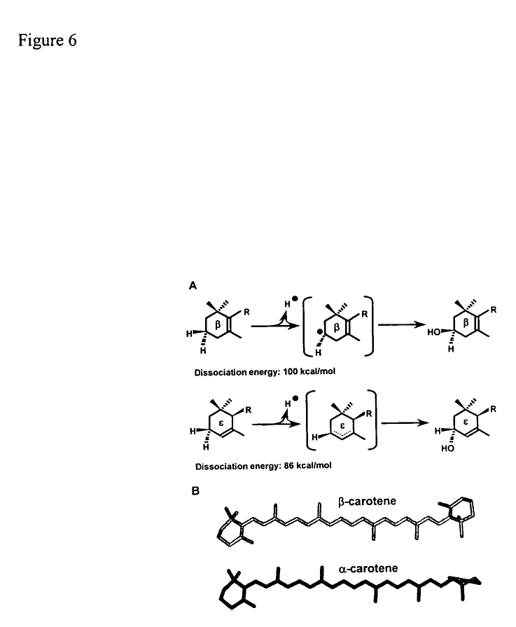

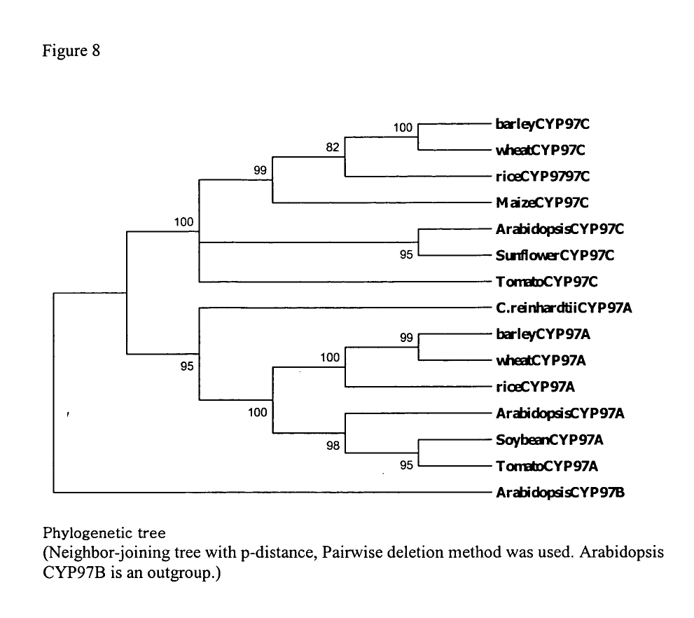

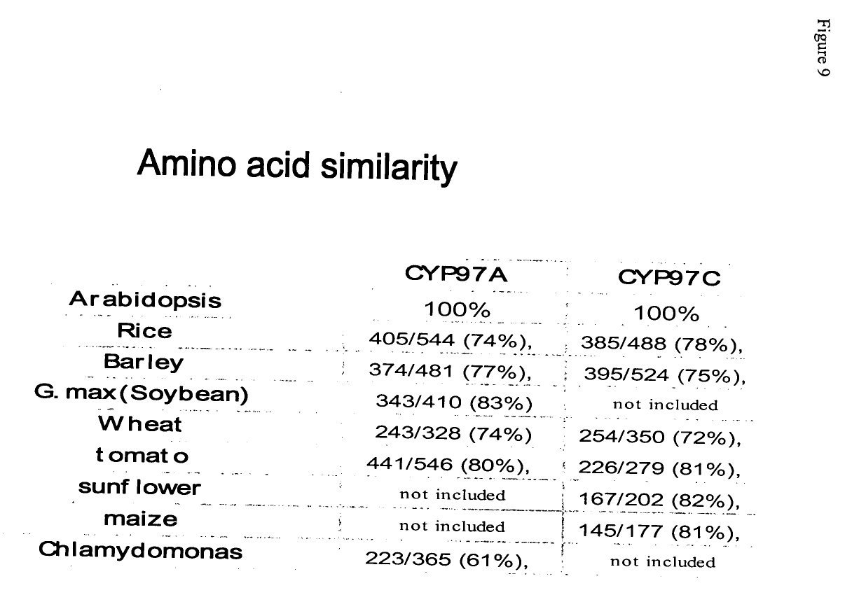

Our Arabidopsis LUT1 sequence was previously designated as CYP97C1 according to the standardized cytochrome P450 nomenclature (http://www.biobase.dk/P450). The Arabidopsis genome also contains two other CYP97 family members, CYP97A3 and CYP97B3, which are 49% and 42% identical to the LUT1 polypeptide, respectively. Interestingly, CYP97A3 (At1g31800) is also one of the nine cytochrome P450s in Arabidopsis predicted to be chloroplast-targeted, while CYP97B3 (At4g15110) is predicted to be targeted to the mitochondria (Schuler and Werck-Reichhart, Annu. Rev. Plant Biol. 54, 629-667 (2003), herein incorporated by reference). Additional CYP97 family proteins were identified in the EST and genomic databases from a wide variety of monocots and dicots, including Arabidopsis, barley, rice, wheat, soybean, pea, sunflower, tomato, and diatom (FIGS. 5 and 8). The term “EST” and “expressed sequence tag” refers to a unique stretch of DNA within a coding region of a gene; approximately 200 to 600 base pairs in length. The term “contig” refers to an overlapping collection of sequences or clones.

V. Cytochrome P450 Genes, Coding Sequences and Polypeptides

A. Nucleic Acid Sequences

1. Arabidopsis LUT1 CYP97C Genes

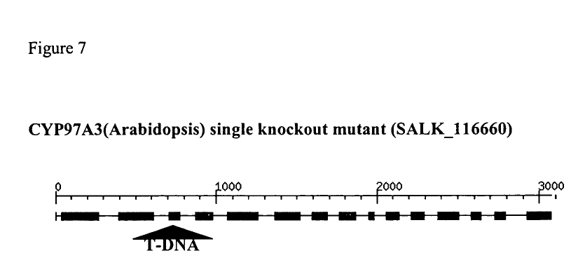

The present invention provides plant LUT1 genes and proteins including their homologs, orthologs, paralogs, variants and mutants. The designation “LUT” refers to the phenotype exhibited by plants with a mutation in a LUT1 gene (the mutant allele is termed lut1), where the mutant has lowered levels of lutein (also referred to as decreased ε-ring hydroxylase activity). In some embodiments of the present invention, isolated nucleic acid sequences comprising LUT1 genes are provided. Mutations in these genes, which disrupt expression of the genes, result in altered carotenoid ratios and carotenoid phenotype. In some embodiments, isolated nucleic acid sequences comprising lut1-1, lut1-2, lut1-3 or CYP97C or CYP97B are provided. These sequences include sequences comprising lut1 and CYP97C cDNA/genomic sequences (for example, as shown in FIGS. 2B, 2C and FIG. 7; SEQ ID NOs: 7-9 and 58).

2. Additional Arabidopsis CYP97A and CYP97B Genes

The present invention provides nucleic acid sequences comprising additional CYP97 cytochrome P450 genes. For example, some embodiments of the present invention provide polynucleotide sequences that produce polypeptides that are homologous to at least one of SEQ ID NOs: 1-3. In some embodiments, the polypeptides are at least 40%, 60%, 70%, 80%, 90%, 95% (or more) identical to any of SEQ ID NOs: 1-4, 16-21, 33-39, 49-52 and 56. Other embodiments of the present invention provide sequences assembled through EST sequences that produce polypeptides at least 40% or more (e.g., 60%, 70%, 80%, 90%, 95%) identical to at least one of SEQ ID NOs: 11-14, 16-21, 33-39, 49-52 and 56. In other embodiments, the present invention provides nucleic acid sequences that hybridize under conditions ranging from low to high stringency to at least one of SEQ ID NOs: 5-9, 22-27, 40-48, 53-56, and 58, as long as the polynucleotide sequence capable of hybridizing to at least one of SEQ ID NOs: 5-9, 22-27, 40-48, 53-55, 57 and 58 encodes a protein that retains a desired biological activity of a carotenoid hydroxylase protein; in some preferred embodiments, the hybridization conditions are high stringency. In preferred embodiments, hybridization conditions are based on the melting temperature (Tₘ) of the nucleic acid binding complex and confer a defined “stringency” as explained above (See e.g., Wahl et al., Meth. Enzymol., 152:399-407 (1987), incorporated herein by reference).

In other embodiments of the present invention, alleles of CYP97 hydroxylase genes, and in particular of CYP97 genes, are provided. In preferred embodiments, alleles result from a mutation, (i.e., a change in the nucleic acid sequence) and generally produce altered mRNAs or polypeptides whose structure or function may or may not be altered.

Any given gene may have none, one or many allelic forms. Common mutational changes that give rise to alleles are generally ascribed to deletions, additions, or insertions, or substitutions of nucleic acids. Each of these types of changes may occur alone, or in combination with the others, and at the rate of one or more times in a given sequence. Mutational changes in alleles also include rearrangements, insertions, deletions, additions, or substitutions in upstream regulatory regions. In one embodiment, a T-DNA insertion element disrupts the expression of a CYP97 gene.

In other embodiments of the present invention, the polynucleotide sequence encoding a CYP97 gene is extended utilizing the nucleotide sequences (e.g., SEQ ID NOs: 5-9, 22-27, 40-48, 53-55, 57 and 58 ) in various methods known in the art to detect upstream sequences such as promoters and regulatory elements. For example, it is contemplated that for LUT1, lut1-1, lut1-2, lut1-3, or related CYP97 hydroxylases, the sequences upstream are identified from the Arabidopsis genomic database. For other lut1 genes for which a database is available, the sequences upstream of the identified lut1 genes can also be identified. An example of an allele for an upstream region is shown is described herein as lut1-2 (SEQ ID NO: 8). For other lut1 and CYP97 genes for which a public genomic database is not available, or not complete, it is contemplated that polymerase chain reaction (PCR) finds use in the present invention.

In another embodiment, inverse PCR is used to amplify or extend sequences using divergent primers based on a known region (Triglia et al., Nucleic Acids Res., 16:8186 (1988), herein incorporated by reference). In yet another embodiment of the present invention, capture PCR (Lagerstrom et al., PCR Methods Applic., 1:111-19 (1991), herein incorporated by reference) is used. In still other embodiments, walking PCR is utilized. Walking PCR is a method for targeted gene walking that permits retrieval of unknown sequence (Parker et al., Nucleic Acids Res., 19:3055-60 (1991), herein incorporated by reference). The PROMOTERFINDER kit (Clontech) uses PCR, nested primers and special libraries to “walk in” genomic DNA. This process avoids the need to screen libraries and is useful in finding intron/exon junctions. In yet other embodiments of the present invention, add TAIL PCR is used as a preferred method for obtaining flanking genomic regions, including regulatory regions (Lui and Whittier, (1995); Lui et al., (1995), herein incorporated by reference). Preferred libraries for screening for full-length cDNAs include libraries that have been size-selected to include larger cDNAs. Also, random primed libraries are preferred, in that they contain more sequences that contain the 5′ and upstream gene regions. A randomly primed library may be particularly useful in cases where an oligo d(T) library does not yield full-length cDNA. Genomic Libraries are useful for obtaining introns and extending 5′ sequence.

3. Variant lut1 Genes

In some embodiments, the present invention provides isolated variants of the disclosed nucleic acid sequences encoding CYP97 genes, and in particular of lut1, lut1-1, lut1-2, lut1-3, or related P450-like hydroxylases genes, and the polypeptides encoded thereby; these variants include mutants, fragments, fusion proteins or functional equivalents of genes and gene protein products. The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

Thus, nucleotide sequences of the present invention are engineered in order to introduce or alter a LUT1 coding sequence for a variety of reasons, including but not limited to initiating the production of lutein; alterations that modify the cloning, processing and/or expression of the gene product (such alterations include inserting new restriction sites and changing codon preference), as well as varying the protein function activity (such changes include but are not limited to differing binding kinetics to nucleic acid and/or protein or protein complexes or nucleic acid/protein complexes, differing binding inhibitor affinities or effectiveness, differing reaction kinetics, varying subcellular localization, and varying protein processing and/or stability).

a. Mutants. Some embodiments of the present invention provide nucleic acid sequences encoding mutant forms of LUT1 proteins, and in particular of LUT1-1 and LUT 1-3 proteins, (i.e., mutants), and the polypeptides encoded thereby. In preferred embodiments, mutants result from mutation of the coding sequence, (i.e., a change in the nucleic acid sequence) and generally produce altered mRNAs or polypeptides whose structure or function may or may not be altered. Any given gene may have none, one, or many variant forms. Common mutational changes that give rise to variants are generally ascribed to deletions, additions or substitutions of nucleic acids. Each of these types of changes may occur alone, or in combination with the others, and at the rate of one or more times in a given sequence.

Mutants of lut1 genes can be generated by any suitable method well known in the art, including but not limited to EMS induced mutagenesis, site-directed mutagenesis, randomized “point” mutagenesis, and domain-swap mutagenesis in which portions of the lut1 cDNA are “swapped” with the analogous portion of other lut1-encoding cDNAs (Back and Chappell, PNAS 93: 6841-6845, (1996), herein incorporated by reference). For example, mutants of lut1 are provided by EMS induced mutations (Pogson, et al. Plant Cell 8, 1627-1639 (1996), herein incorporated by reference).

It is contemplated that is possible to modify the structure of a peptide having an activity (e.g., such as a hydroxylase activity), for such purposes as increasing synthetic activity or altering the affinity of the LUT1 protein for a binding partner or a kinetic activity. Such modified peptides are considered functional equivalents of peptides having an activity of a LUT1 activity as defined herein. A modified peptide can be produced in which the nucleotide sequence encoding the polypeptide has been altered, such as by substitution, deletion, or addition. In some preferred embodiments of the present invention, the alteration increases or decreases the effectiveness of the lut1 gene product to exhibit a phenotype caused by altered carotenoid production. In other words, construct “X” can be evaluated in order to determine whether it is a member of the genus of modified or variant lut1 genes of the present invention as defined functionally, rather than structurally. Accordingly, in some embodiments the present invention provides nucleic acids comprising a lut1 or CYP97 sequence that complement the coding regions of any of SEQ ID NOs: 5-9, 22-27, 40-48, 53-55, 57 and 58, as well as the polypeptides encoded by such nucleic acids. In some embodiments LUT1 is converted to a β-hydroxylase. In some embodiments CYP97A is converted to an ε-hydroxylase. In some embodiments the location of the hydroxylation on the ring is changed (e.g. from carbon 3 to carbons 2, 4, 5, or 6,). In some embodiments, CYP97A activity is reversed to CYP97B activity. Examples of such substitutions are provided by Cunningham and Gantt E. Proc Natl Acad Sci USA. 27;98(5):2905-10 (2001), herein incorporated by reference.

Moreover, as described above, mutant forms of LUT1 proteins are also contemplated as being equivalent to those peptides that are modified as set forth in more detail herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present invention provide nucleic acids comprising sequences encoding variants of lut1 gene products disclosed herein containing conservative replacements, as well as the proteins encoded by such nucleic acids. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur -containing (cysteine and methionine) (e.g., Stryer ed., Biochemistry, pg. 17-21, 2nd ed, WH Freeman and Co., 1981, herein incorporated by reference). Whether a change in the amino acid sequence of a peptide results in a functional homolog can be readily determined by assessing the ability of the variant peptide to function in a fashion similar to the wild-type protein. Peptides having more than one replacement can readily be tested in the same manner.

More rarely, a mutant includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.). Accordingly, other embodiments of the present invention provide nucleic acids comprising sequences encoding variants of lut1 gene products disclosed herein containing non-conservative replacements where the biological activity of the encoded protein is retained, as well as the proteins encoded by such nucleic acids.

b. Directed Evolution. Variants of lut1 genes or coding sequences may be produced by methods such as directed evolution or other techniques for producing combinatorial libraries of variants. Thus, the present invention further contemplates a method of generating sets of nucleic acids that encode combinatorial mutants of the LUT1 proteins, as well as truncation mutants, and is especially useful for identifying potential variant sequences (i.e., homologs) that possess the biological activity of the encoded LUT1 proteins. In addition, screening such combinatorial libraries is used to generate, for example, novel encoded lut1 gene product homologs that possess novel binding or other kinetic specificities or other biological activities. The invention further provides sets of nucleic acids generated as described above, where a set of nucleic acids encodes combinatorial mutants of the LUT1 proteins, or truncation mutants, as well as sets of the encoded proteins. The invention further provides any subset of such nucleic acids or proteins, where the subsets comprise at least two nucleic acids or at least two proteins.

It is contemplated that LUT1, and in particular lut1, lut1-1, lut1-2, lut1-3, or related P450-like hydroxylases genes; genes and coding sequences (e.g., any one or more of SEQ ID NOs: 5-9, 22-27, 40-48, 53-55, 57 and 58 and fragments and variants thereof) can be utilized as starting nucleic acids for directed evolution. These techniques can be utilized to develop encoded LUT1 product variants having desirable properties such as increased kinetic activity or altered binding affinity.

In some embodiments, artificial evolution is performed by random mutagenesis (e.g., by utilizing error-prone PCR to introduce random mutations into a given coding sequence). This method requires that the frequency of mutation be finely tuned. As a general rule, beneficial mutations are rare, while deleterious mutations are common. This is because the combination of a deleterious mutation and a beneficial mutation often results in an inactive enzyme. The ideal number of base substitutions for targeted gene is usually between 1.5 and 5 (Moore and Arnold, Nat. Biotech., 14, 458-67 (1996); Leung et al., Technique, 1:11-15 (1989); Eckert and Kunkel, PCR Methods Appl., 1:17-24 (1991); Caldwell and Joyce, PCR Methods Appl., 2:28-33 (1992); and Zhao and Arnold, Nuc. Acids. Res., 25:1307-08 (1997, all of which are herein incorporated by reference).

After mutagenesis, the resulting clones are selected for desirable activity (e.g., screened for abolishing or restoring hydroxylase activity in a constitutive mutant, in a wild type background where hydroxylase activity is required, as described above and below). Successive rounds of mutagenesis and selection are often necessary to develop enzymes with desirable properties. It should be noted that only the useful mutations are carried over to the next round of mutagenesis.

In other embodiments of the present invention, the polynucleotides of the present invention are used in gene shuffling or special PCR procedures (e.g., Smith, Nature, 370:324-25 (1994); U.S. Pat. Nos. 5,837,458; 5,830,721; 5,811,238; 5,733,731, all of which are herein incorporated by reference). Gene shuffling involves random fragmentation of several mutant DNAs followed by their reassembly by PCR into full-length molecules. Examples of various gene shuffling procedures include, but are not limited to, assembly following DNase treatment, the staggered extension process (STEP), and random priming in vitro recombination.

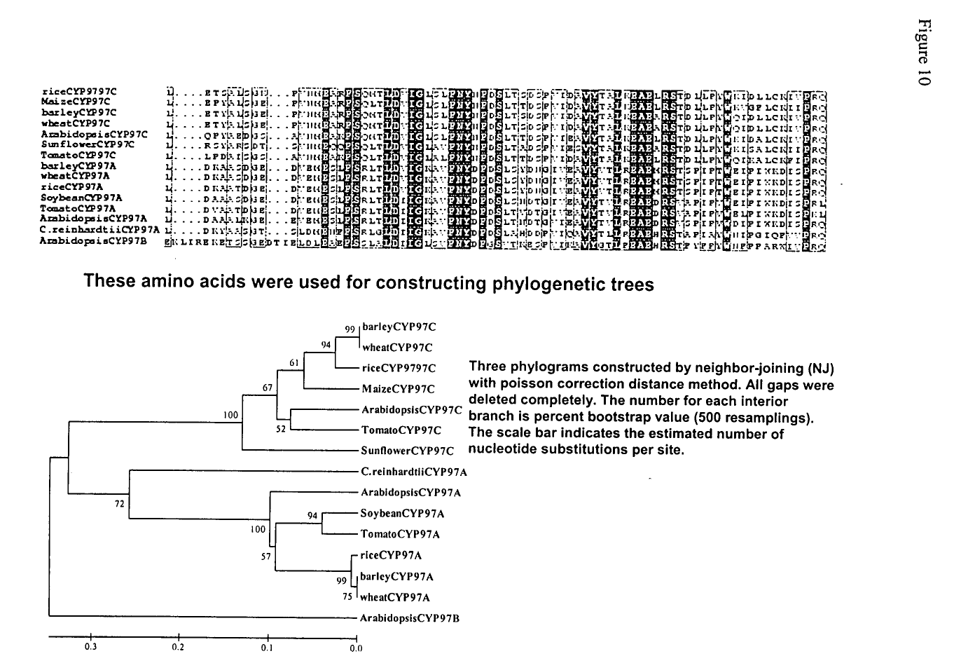

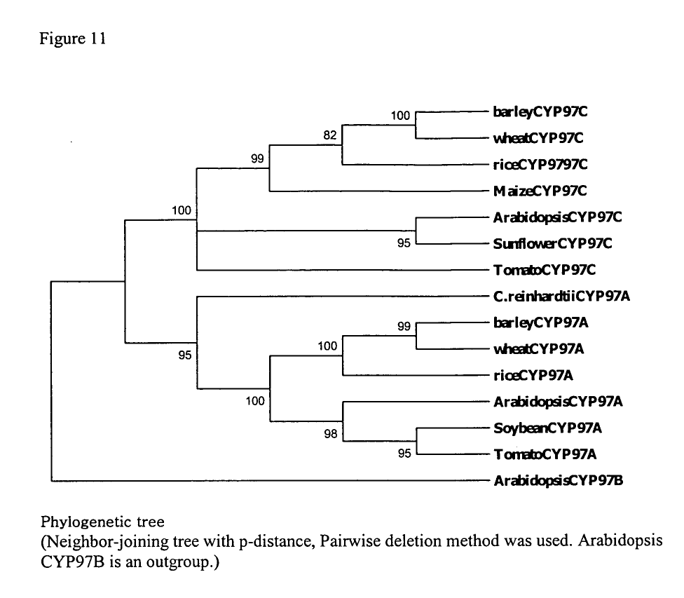

c. Homologs. In some embodiments, the present invention provides isolated variants of the disclosed nucleic acid sequence encoding CYP97 genes, and in particular of lut1, lut1-1, lut1-2, lut1-3, or related P450-like hydroxylases genes, and the polypeptides encoded thereby; these variants include mutants, fragments, fusion proteins or functional equivalents genes and protein products. The term “homology” when used in relation to nucleic acids or proteins refers to a degree of identity. There may be partial homology or complete homology. The following terms are used to describe the sequence relationships between two or more polynucleotides and between two or more polypeptides: “identity,” “percentage identity,” “identical,” “reference sequence”, “sequence identity”, “percentage of sequence identity”, and “substantial identity.” “Sequence identity” refers to a measure of relatedness between two or more nucleic acids or proteins, and is described as a given as a percentage “of homology” with reference to the total comparison length. A “reference sequence” is a defined sequence used as a basis for a sequence comparison; a reference sequence may be a subset of a larger sequence, for example, the sequence that forms an active site of a protein or a segment of a full-length cDNA sequence or may comprise a complete gene sequence. Since two polynucleotides or polypeptides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of in internal region of a polypeptide. In one embodiment, a comparison window is at least 77 amino acids long. In another embodiment, a comparison window is at least 84 amino acids long. In another embodiment, conserved regions of proteins are comparison windows. In a further embodiment, an amino acid sequence for a conserved transmembrane domain is 24 amino acids. An example of a comparison window for a percent homology determination of the present invention is shown in FIG. 10 and described in Example 1. Calculations of identity may be performed by algorithms contained within computer programs such as the ClustalX algorithm (Thompson, et al. Nucleic Acids Res. 24, 4876-4882 (1997), herein incorporated by reference); MEGA2 (version 2.1) (Kumar, et al. Bioinformatics 17, 1244-1245 (2001);“GAP” (Genetics Computer Group, Madison, Wis.) and “ALIGN” (DNAStar, Madison, Wis., all of which are herein incorporated by reference).

For comparisons of nucleic acids, 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math. 2: 482 (1981)) by the homology alignment algorithm of Needleman and Wunsch (Needleman and Wunsch, J. Mol. Biol. 48:443 (1970), herein incorporated by reference), by the search for similarity method of Pearson and Lipman (Pearson and Lipman, Proc. Natl. Acad. Sci. (U.S.A.) 85:2444 (1988), herein incorporated by reference), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide or two polypeptide sequences are identical (i.e., on a nucleotide-by-nucleotide basis or amino acid basis) over the window of comparison. The term “percentage of sequence identity” is calculated by comparing two optimally aligned sequences over the window of comparison, determining the number of positions at which the identical nucleic acid base (e.g., A, T, C, G, U, or 1) or amino acid, in which often conserved amino acids are taken into account, occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

Some homologs of encoded CYP97 products have intracellular half-lives dramatically different than the corresponding wild-type protein. For example, the altered protein is rendered either more stable or less stable to proteolytic degradation or other cellular process that result in destruction of, or otherwise inactivate the encoded CYP97 product. Such homologs, and the genes that encode them, can be utilized to alter the activity of the encoded CYP97 products by modulating the half-life of the protein. For instance, a short half-life can give rise to more transient CYP97 biological effects. Other homologs have characteristics which are either similar to wild-type CYP97, or which differ in one or more respects from wild-type CYP97.

In some embodiments of the combinatorial mutagenesis approach of the present invention, the amino acid sequences for a population of LUT1 gene product homologs are aligned, preferably to promote the highest homology possible. Such a population of variants can include, for example, LUT1 gene homologs from one or more species, or lut1 gene homologs from the same species but which differ due to mutation. Amino acids that appear at each position of the aligned sequences are selected to create a degenerate set of combinatorial sequences.

In a preferred embodiment of the present invention, the combinatorial LUT1 gene library is produced by way of a degenerate library of genes encoding a library of polypeptides that each include at least a portion of candidate encoded LUT1-protein sequences. For example, a mixture of synthetic oligonucleotides is enzymatically ligated into gene sequences such that the degenerate set of candidate LUT1 sequences are expressible as individual polypeptides, or alternatively, as a set of larger fusion proteins (e.g., for phage display) containing the set of LUT1 sequences therein.

There are many ways by which the library of potential LUT1 homologs can be generated from a degenerate oligonucleotide sequence. In some embodiments, chemical synthesis of a degenerate gene sequence is carried out in an automatic DNA synthesizer, and the synthetic genes are ligated into an appropriate gene for expression. The purpose of a degenerate set of genes is to provide, in one mixture, all of the sequences encoding the desired set of potential LUT1 sequences or any combination of CYP97A sequences and CYP97B sequences. The synthesis of degenerate oligonucleotides is well known in the art (See e.g., Narang, Tetrahedron Lett., 39:3 9 (1983); Itakura et al., Recombinant DNA, in Walton (ed.), Proceedings of the 3rd Cleveland Symposium on Macromolecules, Elsevier, Amsterdam, pp 273-289 (1981); Itakura et al., Annu. Rev. Biochem., 53:323 (1984); Itakura et al., Science 198:1056 (1984); Ike et al., Nucl. Acid Res., 11:477 (1983), all of which are herein incorporated by reference). Such techniques have been employed in the directed evolution of other proteins (See e.g., Scott et al., Science, 249:386-390 (1980); Roberts et al., Proc. Natl. Acad. Sci. USA, 89:2429-2433 (1992); Devlin et al., Science, 249: 404-406 (1990); Cwirla et al., Proc. Natl. Acad. Sci. USA, 87: 6378-6382 (1990); as well as U.S. Pat. Nos. 5,223,409, 5,198,346, and 5,096,815, all of which are herein incorporated by reference).

d. Screening Gene Products. A wide range of techniques are known in the art for screening gene products of combinatorial libraries made by point mutations, and for screening cDNA libraries for gene products having a certain property. Such techniques are generally adaptable for rapid screening of the gene libraries generated by the combinatorial mutagenesis of LUT1 and/or CYP97A orthologs. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

Accordingly, in some embodiments of the present invention, the gene library is cloned into the gene for a surface membrane protein of a bacterial cell, and the resulting fusion protein detected by panning (WO 88/06630; Fuchs et al., BioTechnol., 9:1370-1371 (1991); and Goward et al., TIBS 18:136-140 (1992), all of which are herein incorporated by reference. In other embodiments of the present invention, fluorescently labeled molecules that bind encoded LUT1 products can be used to score for potentially functional LUT1 and/or CYP97A orthologs. Cells are visually inspected and separated under a fluorescence microscope, or, where the morphology of the cell permits, separated by a fluorescence-activated cell sorter.

In an alternate embodiment of the present invention, the gene library is expressed as a fusion protein on the surface of a viral particle. For example, foreign peptide sequences are expressed on the surface of infectious phage in the filamentous phage system, thereby conferring two significant benefits. First, since these phages can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (See e.g., WO 90/02909; WO 92/09690; Marks et al., J. Biol. Chem., 267:16007-16010 (1992); Griffths et al., EMBO J., 12:725-734 (1993); Clackson et al., Nature, 352:624-628 (1991); and Barbas et al., Proc. Natl. Acad. Sci., 89:4457-4461 (1992), all of which are herein incorporated by reference).

In another embodiment of the present invention, the recombinant phage antibody system (e.g., RPAS, Pharmacia Catalog number 27-9400-01) is modified for use in expressing and screening of encoded LUT1 and/or CYP97A ortholog product combinatorial libraries. The pCANTAB 5 phagemid of the RPAS kit contains the gene that encodes the phage gIII coat protein. In some embodiments of the present invention, the LUT1 and/or CYP97A ortholog combinatorial gene library is cloned into the phagemid adjacent to the gIII signal sequence such that it is expressed as a gIII fusion protein. In other embodiments of the present invention, the phagemid is used to transform competent E. coli TG1 cells after ligation. In still other embodiments of the present invention, transformed cells are subsequently infected with M13KO7 helper phage to rescue the phagemid and its candidate lut1 gene insert. The resulting recombinant phage contain phagemid DNA encoding a specific candidate LUT1 protein and display one or more copies of the corresponding fusion coat protein. In some embodiments of the present invention, the phage-displayed candidate proteins that display any property characteristic of a LUT1 protein are selected or enriched by panning. The bound phage is then isolated, and if the recombinant phages express at least one copy of the wild type gIII coat protein, they will retain their ability to infect E. coli. Thus, successive rounds of reinfection of E. coli and panning will greatly enrich for LUT1 and/or CYP97A orthologs.

In light of the present disclosure, other forms of mutagenesis generally applicable will be apparent to those skilled in the art in addition to the aforementioned rational mutagenesis based on conserved versus non-conserved residues. For example, LUT1 homologs can be generated and screened using, for example, alanine scanning mutagenesis and the like (Ruf et al., Biochem., 33:1565-1572 (1994); Wang et al., J. Biol. Chem., 269:3095-3099 (1994); Balint Gene 137:109-118 (1993); Grodberg et al., Eur. J. Biochem., 218:597-601 (1993); Nagashima et al., J. Biol. Chem., 268:2888-2892 (1993); Lowman et al., Biochem., 30:10832-10838 (1991); and Cunningham et al., Science, 244:1081-1085 (1989), all of which are herein incorporated by reference), by linker scanning mutagenesis (Gustin et al., Virol., 193:653-660 (1993); Brown et al., Mol. Cell. Biol., 12:2644-2652 (1992); McKnight et al., Science, 232:316), or by saturation mutagenesis (Meyers et al., Science, 232:613 (1986), all of which are herein incorporated by reference).

e. Truncation Mutants of LUT1 and/or CYP97A orthologs. In addition, the present invention provides isolated nucleic acid sequences encoding fragments of encoded LUT1 and/or CYP97A ortholog products (i.e., truncation mutants), and the polypeptides encoded by such nucleic acid sequences. In preferred embodiments, the LUT1 fragment is biologically active. An example of a truncation unit resulting from mistranslation is described herein as lut1-1. In some embodiments of the present invention, when expression of a portion of a LUT1 and/or CYP97A ortholog protein is desired, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al., J. Bacteriol., 169:751-757 (1987), herein incorporated by reference) and Salmonella typhimurium and its in vitro activity has been demonstrated on recombinant proteins (Miller et al., Proc. Natl. Acad. Sci. USA, 84:2718-1722 (1990), herein incorporated by reference). Therefore, removal of an N-terminal methionine, if desired, can be achieved either in vivo by expressing such recombinant polypeptides in a host that produces MAP (e.g., E. coli or CM89 or S. cerevisiae), or in vitro by use of purified MAP.

f. Fusion Proteins Containing LUT1 and/or CYP97A orthologs. The present invention also provides nucleic acid sequences encoding fusion proteins incorporating all or part of LUT1 and/or CYP97A orthologs, and the polypeptides encoded by such nucleic acid sequences. The term “fusion” when used in reference to a polypeptide refers to a chimeric protein containing a protein of interest joined to an exogenous protein fragment (the fusion partner). The term “chimera” when used in reference to a polypeptide refers to the expression product of two or more coding sequences obtained from different genes, that have been cloned together and that, after translation, act as a single polypeptide sequence. Chimeric polypeptides are also referred to as “hybrid” polypeptides. The coding sequences include those obtained from the same or from different species of organisms. The fusion partner may serve various functions, including enhancement of solubility of the polypeptide of interest, as well as providing an “affinity tag” to allow purification of the recombinant fusion polypeptide from a host cell or from a supernatant or from both. If desired, the fusion partner may be removed from the protein of interest after or during purification. In some embodiments, the fusion proteins have a LUT1 and/or a CYP97A ortholog functional domain with a fusion partner. Accordingly, in some embodiments of the present invention, the coding sequences for the polypeptide (e.g., a LUT1 functional domain) is incorporated as a part of a fusion gene including a nucleotide sequence encoding a different polypeptide. It is contemplated that such a single fusion product polypeptide is able to enhance hydroxylase activity, such that the transgenic plant produces altered carotenoid ratios.

In some embodiments of the present invention, chimeric constructs code for fusion proteins containing a portion of a LUT1 and/or CYP97A ortholog protein and a portion of another gene. In some embodiments, the fusion proteins have biological activity similar to the wild type LUT1 (e.g., have at least one desired biological activity of a LUT1 protein). In other embodiments, the fusion protein has altered biological activity.

In addition to utilizing fusion proteins to alter biological activity, it is widely appreciated that fusion proteins can also facilitate the expression and/or purification of proteins, such as the LUT1 and/or CYP97A ortholog protein of the present invention. Accordingly, in some embodiments of the present invention, a LUT1 protein is generated as a glutathione-S-transferase (i.e., GST fusion protein). It is contemplated that such GST fusion proteins enables easy purification of the LUT1 and/or CYP97A ortholog protein, such as by the use of glutathione-derivatized matrices (See e.g., Ausabel et al. (eds.), Current Protocols in Molecular Biology, John Wiley & Sons, NY (1991), herein incorporated by reference).

In another embodiment of the present invention, a fusion gene coding for a purification leader sequence, such as a poly-(His)/enterokinase cleavage site sequence at the N-terminus of the desired portion of a LUT1 and/or CYP97A ortholog protein allows purification of the expressed LUT1 and/or CYP97A ortholog fusion protein by affinity chromatography using a Ni²⁺ metal resin. In still another embodiment of the present invention, the purification leader sequence is then subsequently removed by treatment with enterokinase (See e.g., Hochuli et al., J. Chromatogr., 411:177 (1987); and Janknecht et al., Proc. Natl. Acad. Sci. USA, 88:8972, all of which are herein incorporated by reference). In yet other embodiments of the present invention, a fusion gene coding for a purification sequence appended to either the N or the C terminus allows for affinity purification; one example is addition of a hexahistidine tag to the carboxy terminus of a LUT1 and/or CYP97A ortholog protein that is optimal for affinity purification.

Techniques for making fusion genes are well known. Essentially, the joining of various nucleic acid fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment of the present invention, the fusion gene can be synthesized by conventional techniques including automated DNA synthesizers. Alternatively, in other embodiments of the present invention, PCR amplification of gene fragments is carried out using anchor primers that give rise to complementary overhangs between two consecutive gene fragments that can subsequently be annealed to generate a chimeric gene sequence (See e.g., Current Protocols in Molecular Biology, supra, herein incorporated by reference).

B. Encoded lut1 Gene Polypeptides

The present invention provides isolated LUT1 and/or CYP97A ortholog polypeptides, as well as variants, homologs, mutants or fusion proteins thereof, as described above. In some embodiments of the present invention, the polypeptide is a naturally purified product, while in other embodiments it is a product of chemical synthetic procedures, and in still other embodiments it is produced by recombinant techniques using a prokaryotic or eukaryotic host (e.g., by bacterial, yeast, higher plant, insect and mammalian cells in culture). In some embodiments, depending upon the host employed in a recombinant production procedure, the polypeptide of the present invention is glycosylated or non-glycosylated. In other embodiments, the polypeptides of the invention also includes an initial methionine amino acid residue.

1. Purification of LUT1 Polypeptides

The present invention provides purified LUT1 and/or CYP97A ortholog polypeptides as well as variants, homologs, mutants or fusion proteins thereof, as described above. In some embodiments of the present invention, LUT1 and/or CYP97A ortholog polypeptides purified from recombinant organisms as described below are provided. In other embodiments, LUT1 and/or CYP97A ortholog polypeptides purified from recombinant bacterial extracts transformed with Arabidopsis LUT1 and/or CYP97A ortholog cDNA, and in particular any one or more of LUT1, and/or CYP97A ortholog and or related P450 monooxygenase cDNA, are provided (as described in the Examples).

The present invention also provides methods for recovering and purifying LUT1 and/or CYP97A orthologs from recombinant cell cultures including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography.

The present invention further provides nucleic acid sequences having the coding sequence (or a portion of the coding sequence) for a LUT1 protein (e.g., SEQ ID NOs: 1-4, 16-21, 33-39, 49-52 and 58 ) and/or CYP97A ortholog protein fused in frame to a marker sequence that allows for expression alone or for both expression and purification of the polypeptide of the present invention. A non-limiting example of a marker sequence is a hexahistidine tag that is supplied by a vector, for example, a pQE-30 vector which adds a hexahistidine tag to the N terminal of a LUT1 gene and/or CYP97A ortholog gene and which results in expression of the polypeptide in a bacterial host, or, for example, the marker sequence is a hemagglutinin (HA) tag when a mammalian host is used. The HA tag corresponds to an epitope derived from the influenza hemagglutinin protein (Wilson et al., Cell, 37:767 (1984), herein incorporated by reference).

2. Chemical Synthesis of LUT1 and/or CYP97A ortholog Polypeptides

In an alternate embodiment of the invention, the coding sequence of LUT1 genes and/or CYP97A ortholog genes, and in particular of any one or more of LUT1, and/or CYP97A orthologs, or related P450 monooxygenase genes, is synthesized, in whole or in part, using chemical methods well known in the art (See e.g., Caruthers et al., Nucl. Acids Res. Symp. Ser., 7:215-233 (1980); Crea and Horn, Nucl. Acids Res., 9:2331 (1980); Matteucci and Caruthers, Tetrahedron Lett., 21:719 (1980); and Chow and Kempe, Nucl. Acids Res., 9:2807-2817 (1981), all of which are herein incorporated by reference). In other embodiments of the present invention, the protein itself is produced using chemical methods to synthesize either an entire LUT1 and/or CYP97A ortholog amino acid sequence (for example, SEQ ID NOs: 4 and/or 33) or a portion thereof. For example, peptides are synthesized by solid phase techniques, cleaved from the resin, and purified by preparative high performance liquid chromatography (See e.g., Creighton, Proteins Structures And Molecular Principles, W.H. Freeman and Co, New York N.Y. (1983), herein incorporated by reference). In other embodiments of the present invention, the composition of the synthetic peptides is confirmed by amino acid analysis or sequencing (See e.g., Creighton, supra, herein incorporated by reference).

Direct peptide synthesis can be performed using various solid-phase techniques (Roberge et al., Science, 269:202-204 (1995), herein incorporated by reference) and automated synthesis may be achieved, for example, using ABI 431A Peptide Synthesizer (Perkin Elmer) in accordance with the instructions provided by the manufacturer. Additionally, the amino acid sequence of LUT1 and/or CYP97A orthologs, or any part thereof, may be altered during direct synthesis and/or combined using chemical methods with other sequences to produce a variant polypeptide.

3. Generation of LUT1 and CYP97A Antibodies

In some embodiments of the present invention, antibodies are generated to allow for the detection and characterization of a LUT1 protein and/or CYP97A ortholog proteins. The antibodies may be prepared using various immunogens. In one embodiment, the immunogen is an Arabidopsis LUT1 peptide (e.g., an amino acid sequence as depicted in SEQ ID NOs: 1-4, 16-21, 33-39, 49-52 and 56), or CYP97A ortholog, or a fragment thereof, to generate antibodies that recognize a plant LUT1 and/or CYP97A ortholog protein. Such antibodies include, but are not limited to polyclonal, monoclonal, chimeric, single chain, Fab fragments, and Fab expression libraries.

Various procedures known in the art may be used for the production of polyclonal antibodies directed against a LUT1 protein. For the production of antibody, various host animals can be immunized by injection with the peptide corresponding to the LUT1 protein and/or CYP97A ortholog protein epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies directed toward a LUT1 protein and/or CYP97A ortholog protein, it is contemplated that any technique that provides for the production of antibody molecules by continuous cell lines in culture finds use with the present invention (See e.g., Harlow and Lane, Antibodies: A Laboratory Manual, Cold Spring Harbor Laboratory Press, Cold Spring Harbor, N.Y., herein incorporated by reference). These include but are not limited to the hybridoma technique originally developed by Köhler and Milstein (Köhler and Milstein, Nature, 256:495-497 (1975), herein incorporated by reference), as well as the trioma technique, the human B-cell hybridoma technique (See e.g., Kozbor et al., Immunol. Tod., 4:72 (1983), herein incorporated by reference), and the EBV-hybridoma technique to produce human monoclonal antibodies (Cole et al., in Monoclonal Antibodies and Cancer Therapy, Alan R. Liss, Inc., pp. 77-96 (1985), herein incorporated by reference).

In an additional embodiment of the invention, monoclonal antibodies are produced in germ-free animals utilizing technology such as that described in PCT/US90/02545). Furthermore, it is contemplated that human antibodies may be generated by human hybridomas (Cote et al., Proc. Natl. Acad. Sci. USA, 80:2026-2030 (1983), herein incorporated by reference) or by transforming human B cells with EBV virus in vitro (Cole et al., in Monoclonal Antibodies and Cancer Therapy, Alan R. Liss, pp. 77-96 (1985), herein incorporated by reference).

In addition, it is contemplated that techniques described for the production of single chain antibodies (U.S. Pat. No. 4,946,778, herein incorporated by reference) find use in producing a LUT1 and/or CYP97A ortholog protein-specific single chain antibodies. An additional embodiment of the invention utilizes the techniques described for the construction of Fab expression libraries (Huse et al., Science, 246:1275-1281 (1989), herein incorporated by reference) to allow rapid and easy identification of monoclonal Fab fragments with the desired specificity for a LUT1 and/or CYP97A ortholog protein.

It is contemplated that any technique suitable for producing antibody fragments finds use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody is accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention. As is well known in the art, the immunogenic peptide should be provided free of the carrier molecule used in any immunization protocol. For example, if the peptide was conjugated to KLH, it may be conjugated to BSA, or used directly, in a screening assay. In some embodiments of the present invention, the foregoing antibodies are used in methods known in the art relating to the expression of a LUT1 protein (e.g., for Western blotting), measuring levels thereof in appropriate biological samples, etc. The antibodies can be used to detect a LUT1 and/or CYP97A ortholog protein in a biological sample from a plant. The biological sample can be an extract of a tissue, or a sample fixed for microscopic examination.

The biological samples are then be tested directly for the presence of a LUT1 and/or CYP97A ortholog protein using an appropriate strategy (e.g., ELISA or radioimmunoassay) and format (e.g., microwells, dipstick (e.g., as described in WO 93/03367 herein incorporated by reference), etc. Alternatively, proteins in the sample can be size separated (e.g., by polyacrylamide gel electrophoresis (PAGE), in the presence or not of sodium dodecyl sulfate (SDS), and the presence of a LUT1 and/or CYP97A ortholog protein detected by immunoblotting (Western blotting). Immunoblotting techniques are generally more effective with antibodies generated against a peptide corresponding to an epitope of a protein, and hence, are particularly suited to the present invention.

C. Expression of Cloned LUT1 and/or CYP97 Genes

In other embodiments of the present invention, nucleic acid sequences corresponding to the LUT1 genes, CYP97 genes, their homologs, orthologs, paralogs, and mutants are provided as described above. The term “homology” when used in relation to nucleic acids or proteins refers to a degree of identity. There may be partial homology or complete homology. The terms “homolog,” “homologue,”” “homologous,” and “homology” when used in reference to amino acid sequence or nucleic acid sequence or a protein or a polypeptide refers to a degree of sequence identity to a given sequence, or to a degree of similarity between conserved regions, or to a degree of similarity between three-dimensional structures or to a degree of similarity between the active site, or to a degree of similarity between the mechanism of action, or to a degree of similarity between functions. In some embodiments, a homolog has a greater than 20% sequence identity to a given sequence. In some embodiments, a homolog has a greater than 40% sequence identity to a given sequence. In some embodiments, a homolog has a greater than 60% sequence identity to a given sequence. In some embodiments, a homolog has a greater than 70% sequence identity to a given sequence. In some embodiments, a homolog has a greater than 90% sequence identity to a given sequence. In some embodiments, a homolog has a greater than 95% sequence identity to a given sequence. In some embodiments, homology is determined by comparing internal conserved sequences to a given sequence. In some embodiments, homology is determined by comparing designated conserved functional regions. In some embodiments, means of determining homology are described in the Experimental section.

The term “ortholog” refers to a gene in different species that evolved from a common ancestral gene by speciation. In some embodiments, orthologs retain the same function. The term “paralog” refers to genes related by duplication within a genome. In some embodiments, paralogs evolve new functions. In further embodiments, a new function of a paralog is related to the original function.

In some embodiments, homologs may be used to generate recombinant DNA molecules that direct the expression of the encoded protein product in appropriate host cells. The term “recombinant” when made in reference to a nucleic acid molecule refers to a nucleic acid molecule that is comprised of segments of nucleic acid joined together by means of molecular biological techniques. The term “recombinant” when made in reference to a protein or a polypeptide refers to a protein molecule that is expressed using a recombinant nucleic acid molecule.

As will be understood by those of skill in the art, it may be advantageous to produce LUT1-encoding nucleotide sequences possessing non-naturally occurring codons. Therefore, in some preferred embodiments, codons preferred by a particular prokaryotic or eukaryotic host (Murray et al., Nucl. Acids Res., 17 (1989), herein incorporated by reference) can be selected, for example, to increase the rate of LUT1 expression or to produce recombinant RNA transcripts having desirable properties, such as a longer half-life, than transcripts produced from naturally occurring sequence.

1. Vectors for Production of LUT1 and/or CYP97A Orthologs

The nucleic acid sequences of the present invention may be employed for producing polypeptides by recombinant techniques. Thus, for example, the nucleic acid sequence may be included in any one of a variety of expression vectors for expressing a polypeptide. The terms “expression vector” or “expression cassette” refer to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

In some embodiments of the present invention, vectors include, but are not limited to, chromosomal, nonchromosomal and synthetic DNA sequences (e.g., derivatives of plant tumor sequences, T-DNA sequences, derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

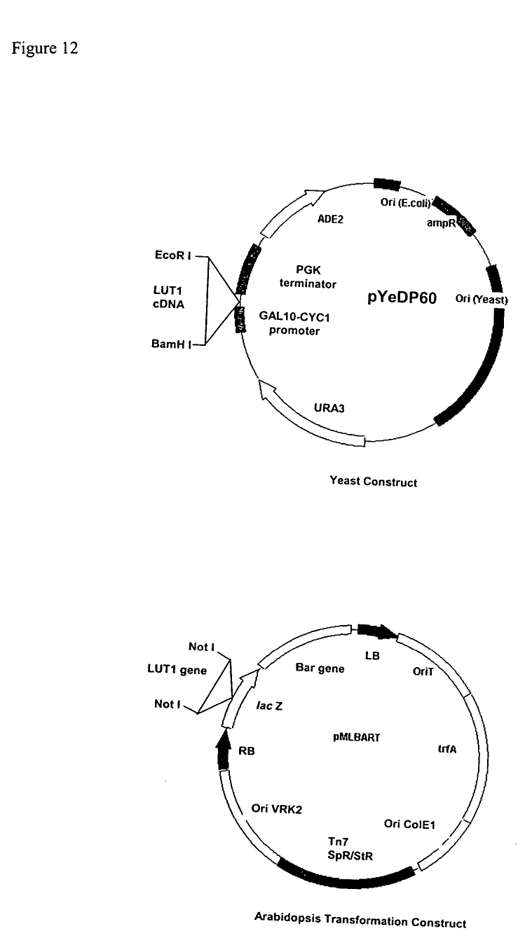

In particular, some embodiments of the present invention provide recombinant constructs comprising one or more of the nucleic sequences as broadly described above (e.g., SEQ ID NOs: 5-9, 22-27, 40-48, 53-55, 57 and 58). In some embodiments of the present invention, the constructs comprise a vector, such as a plasmid or eukaryotic vector, or viral vector, into which a nucleic acid sequence of the invention has been inserted, in a forward or reverse orientation. Examples of such vectors of the present invention are shown in FIG. 12. In preferred embodiments of the present invention, the appropriate nucleic acid sequence is inserted into the vector using any of a variety of procedures. In general, the nucleic acid sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial—pYeDP60, pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia); and 2) Eukaryotic—pMLBART, Agrobacterium tumefaciens strain GV3101, pSV2CAT, pOG44, PXT1, pSG (Stratagene) pSVK3, pBPV, pMSG, and pSVL (Pharmacia). Any other plasmid or vector may be used as long as they are replicable and viable in the host.

In some preferred embodiments of the present invention, plant expression vectors comprise an origin of replication, a suitable promoter and enhancer, and also any necessary ribosome binding sites, polyadenylation sites, splice donor and acceptor sites, transcriptional termination sequences, and 5′ flanking nontranscribed sequences for expression in plants. In other embodiments, DNA sequences derived from the SV40 splice, and polyadenylation sites may be used to provide the required nontranscribed genetic elements.

In certain embodiments of the present invention, the nucleic acid sequence in the expression vector is operatively linked to an appropriate expression control sequence(s) (promoter) to direct mRNA synthesis. Promoters useful in the present invention include, but are not limited to, the LTR or SV40 promoter, the E. coli lac or trp, the phage lambda P_{L} and P_{R}, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (e.g., dihydrofolate reductase or neomycin resistance for eukaryotic cell culture, or tetracycline or ampicillin resistance in E. coli).

In some embodiments of the present invention, transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin bp 100 to 270, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

2. Host Cells for Production of LUT1

In a further embodiment, the present invention provides host cells containing the above-described constructs. The term “host cell” refers to any cell capable of replicating and/or transcribing and/or translating a heterologous gene. Thus, a “host cell” refers to any eukaryotic or prokaryotic cell (e.g., plant cells, algal cells such as C. reinhardtii, bacterial cells such as E. coli, yeast cells, mammalian cells, avian cells, amphibian cells, fish cells, and insect cells), whether located in vitro or in vivo. For example, host cells may be located in a transgenic plant. In some embodiments of the present invention, the host cell is a higher eukaryotic cell (e.g., a plant cell). In other embodiments of the present invention, the host cell is a lower eukaryotic cell (e.g., a yeast cell). The terms “eukaryotic” and “eukaryote” are used in it broadest sense. It includes, but is not limited to, any organisms containing membrane bound nuclei and membrane bound organelles. Examples of eukaryotes include but are not limited to animals, plants, alga, diatoms, and fungi.

In still other embodiments of the present invention, the host cell can be a prokaryotic cell (e.g., a bacterial cell). The terms “prokaryote” and “prokaryotic” are used in it broadest sense. It includes, but is not limited to, any organisms without a distinct nucleus. Examples of prokaryotes include but are not limited to bacteria, blue-green algae, archaebacteria, actinomycetes and mycoplasma. In some embodiments, a host cell is any microorganism. As used herein the term “microorganism” refers to microscopic organisms and taxonomically related macroscopic organisms within the categories of algae, bacteria, fungi (including lichens), protozoa, viruses, and subviral agents. Specific examples of host cells include, but are not limited to, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, (Gluzman, Cell 23:175 (1981), herein incorporated by reference), 293T, C127, 3T3, HeLa and BHK cell lines, NT-1 (tobacco cell culture line), root cell and cultured roots in rhizosecretion (Gleba et al., Proc Natl Acad Sci USA 96: 5973-5977 (1999), herein incorporated by reference). Examples of host cells for carotenoid production are described in U.S. Pat. No. 5,744,341 to Cunningham, et al. (Jul. 4, 1995), herein described by reference.

The constructs in host cells can be used in a conventional manner to produce the gene product encoded by the recombinant sequence. In some embodiments, introduction of the construct into the host cell can be accomplished by calcium phosphate transfection, DEAE-Dextran mediated transfection, or electroporation (See e.g., Davis et al., Basic Methods in Molecular Biology, (1986), herein incorporated by reference). Alternatively, in some embodiments of the present invention, the polypeptides of the invention can be synthetically produced by conventional peptide synthesizers.

Proteins can be expressed in eukaryotic cells, yeast, bacteria, or other cells under the control of appropriate promoters. An example of eukaytoic production of lutein is shown in U.S. Patent Appln. Pub. No. 20030207947 A1 to DeSouza et al. (Nov. 6, 2003), herein incorporated by reference. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989), herein incorporated by reference.

In some embodiments of the present invention, following transformation of a suitable host strain and growth of the host strain to an appropriate cell density, the selected promoter is induced by appropriate means (e.g., temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

V. Methods of Modifying Carotenoid Phenotype by Manipulating LUT1 Gene Expression

The present invention also provides methods of using LUT1 and/or CYP97A ortholog genes. In some embodiments, the sequences are used for research purposes. For example, nucleic acid sequences comprising coding sequences of a LUT1 gene and/or CYP97A orthologs, for example any one or more of LUT1, CYP97A, CYP97B, or related P450 monooxygenases are used to discover other carotenoid synthesis genes. In other embodiments, endogenous plant lut1 genes, such as any one or more of LUT1, CYP97A, CYP97B or related P450 monooxygenases genes, are silenced, for example with antisense RNA, RNAi or by cosuppression, and the effects on carotenoid production observed.

In other embodiments, modifications to nucleic acid sequences encoding CYP97 genes, such as any one or more of LUT1, CYP97A, CYP97B or related related P450 monooxygenase genes, are made, and the effects observed in vivo; for example, modified nucleic sequences encoding at least one LUT1 gene are utilized to transform plants in which endogenous LUT1 genes are silenced by antisense RNA technology, cosuppression or RNAi, and the effects observed. In other embodiments, LUT1 genes, either unmodified or modified, are expressed in vitro translation and/or transcription systems, and the interaction of the transcribed and/or translation product with other system components (such as nucleic acids, proteins, lipids, carbohydrates, or any combination of any of these molecules) observed.

In other embodiments, LUT1 gene sequences are utilized to alter carotenoid phenotype, and/or to control the ratio or levels of various carotenoids in a host. In some embodiments, LUT1 sequences alter the production of hydroxylated carotenes. In yet other embodiments, LUT1 gene sequences are utilized to confer a carotenoid phenotype, and/or to decrease a carotenoid phenotype or to increase the production of a particular carotenoid, or to promote the production of novel carotenoid pigments. Examples are described U.S. Pat. No. 6,524,811 to Cunningham, et al. (Feb. 25, 2003), herein incorporated by reference. Thus, it is contemplated that nucleic acids encoding a LUT1 polypeptide of the present invention may be utilized to either increase or decrease the level of LUT1 mRNA and/or protein in transfected cells as compared to the levels in wild-type cells. Examples are described in U.S. Pat. No. 6,642,021; U.S. Patent Appln. Pub. Nos. US 20020102631A1, US 20020086380A1 to Cunningham, Jr., et al., (Nov. 4, 2003; Aug. 1, 2002, respectively), all of which are herein incorporated by reference).

In some embodiments, the present invention provides methods to over-ride a carotenoid phenotype, and/or to promote overproduction of carotenoids, in plants that require carotenoid, by disrupting the function of at least one lut1 gene in the plant. In these embodiments, the function of at least one LUT1 gene is disrupted by any effective technique, including but not limited to antisense, co-suppression, and RNA interference, as is described above and below. An example of using carotenoid RNA antisense mRNAs to cause a plant to preferentially accumulate alpha-carotene; and produce genetically engineered marigold plants which preferentially overproduce a desired carotenoid pigment in the petal is shown in U.S. Pat. No. 6,232,530 and WO 00/32788 to DellaPenna, et al. (May 15, 2001 and Aug. 6, 2000, respectively), all of which are herein incorporated by reference).

In yet other embodiments, the present invention provides methods to alter a carotenoid phenotype and/or add a carotenoid in plants in which carotenoid is not usually found and/or add a novel or rare carotenoid in plants in which carotenoid is not otherwise found, by expression of at least one heterologous LUT1 gene. Thus, in some embodiments, nucleic acids comprising coding sequences of at least one LUT1 gene, for example any one or more of LUT1, are used to transform plants without a pathway for producing a particular carotenoid such as lutein. It is contemplated that some particular plant species or cultivars do not have any LUT1 genes; for these plants, it is necessary to transform a plant with the necessary LUT1 genes required to confer the preferred carotenoid profile phenotype. It is contemplated that other particular plant species or cultivars may possess at least one LUT1 gene; thus, for these plants, it is necessary to transform a plant with those LUT1 genes that can interact with endogenous LUT1 genes in order to confer a preferred carotenoid profile phenotype. An example is shown in U.S. Pat. No. 5,429,939 to Misawa, et al. (Jul. 4, 1995), herein incorporated by reference. Examples of the production of novel or rare carotenoids are described in U.S. Patent Appln. Pub. No. 20030129264A1 and 20030196232A1; WO 03/001901 to Hauptmann, et al. (Jul. 10, 2003 and Oct. 16, 2003, respectively), all of which are herein incorporated by reference.

The presence of lut1 genes in a species or cultivar can be tested by a number of ways, including but not limited to using probes from genomic or cDNA LUT1 coding sequences, or by using antibodies specific to LUT1 polypeptides. The additional lut1 gene(s) needed to confer the desired phenotype can then be transformed into a plant to confer the phenotype. In these embodiments, plants are transformed with LUT1 genes as described above and below. Examples of transformed plants such as marigold are described in U.S. Pat. No. 6,232,530 and WO 00/32788 to DellaPenna, et al. (May 15, 2001 and Aug. 6, 2000, respectively), herein incorporated by reference.

As described above, in some embodiments, it is contemplated that the nucleic acids encoding a LUT1 polypeptide of the present invention may be utilized to decrease the level of LUT1 mRNA and/or protein in transfected cells as compared to the levels in wild-type cells. In some of these embodiments, the nucleic acid sequence encoding a LUT1 protein of the present invention is used to design a nucleic acid sequence encoding a nucleic acid product that interferes with the expression of the nucleic acid encoding a LUT1 polypeptide, where the interference is based upon a coding sequence of the encoded LUT1 polypeptide. Exemplary methods are described further below. An example of mutant marigolds with less lutein than non-mutant marigolds is shown in U.S. Patent Appln. Pub. Nos. 20030129264A1 and 20030196232A1; WO 03/001901 to Hauptmann, et al. (Jul. 10, 2003 and Oct. 16, 2003; Sep. 1, 2003, respectively), all of which are herein incorporated by reference.

One method of reducing LUT1 expression utilizes expression of antisense transcripts. Antisense RNA has been used to inhibit plant target genes in a tissue-specific manner (e.g., van der Krol et al. (1988) Biotechniques 6:958-976, herein incorporated by reference). Antisense inhibition has been shown using the entire cDNA sequence as well as a partial cDNA sequence (e.g., Sheehy et al. (1988) Proc. Natl. Acad. Sci. USA 85:8805-8809; Cannon et al. (1990) Plant Mol. Biol. 15:39-47, herein incorporated by reference). There is also evidence that 3′ non-coding sequence fragment and 5′ coding sequence fragments, containing as few as 41 base-pairs of a 1.87 kb cDNA, can play important roles in antisense inhibition (Ch'ng et al. (1989) Proc. Natl. Acad. Sci. USA 86:10006-10010, herein incorporated by reference).

Accordingly, in some embodiments, a LUT1 encoding-nucleic acid of the present invention are oriented in a vector and expressed so as to produce antisense transcripts. To accomplish this, a nucleic acid segment from the desired gene is cloned and operably linked to a promoter such that the antisense strand of RNA will be transcribed. The expression cassette is then transformed into plants and the antisense strand of RNA is produced. The nucleic acid segment to be introduced generally will be substantially identical to at least a portion of the endogenous gene or genes to be repressed. The sequence, however, need not be perfectly identical to inhibit expression. The vectors of the present invention can be designed such that the inhibitory effect applies to other proteins within a family of genes exhibiting homology or substantial homology to the target gene.

Furthermore, for antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of at least about 500 nucleotides is especially preferred.

Catalytic RNA molecules or ribozymes can also be used to inhibit expression of the target gene or genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs which are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, Solanum nodiflorum mottle virus and subterranean clover mottle virus. The design and use of target RNA-specific ribozymes is described in Haseloff, et al. (1988) Nature 334:585-591. Ribozymes targeted to the mRNA of a lipid biosynthetic gene, resulting in a heritable increase of the target enzyme substrate, have also been described (Merlo A O et al. (1998) Plant Cell 10: 1603-1621, herein incorporated by reference).

Another method of reducing LUT1 expression utilizes the phenomenon of cosuppression or gene silencing (See e.g., U.S. Pat. No. 6,063,947, herein incorporated by reference). The phenomenon of cosuppression has also been used to inhibit plant target genes in a tissue-specific manner. Cosuppression of an endogenous gene using a full-length cDNA sequence as well as a partial cDNA sequence (730 bp of a 1770 bp cDNA) are known (e.g., Napoli et al. (1990) Plant Cell 2:279-289; van der Krol et al. (1990) Plant Cell 2:291-299; Smith et al. (1990) Mol. Gen. Genetics 224:477-481, herein incorporated by reference). Accordingly, in some embodiments the nucleic acid sequences encoding a LUT1 of the present invention are expressed in another species of plant to effect cosuppression of a homologous gene.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect should apply to any other proteins within a similar family of genes exhibiting homology or substantial homology.

For cosuppression, the introduced sequence in the expression cassette, needing less than absolute identity, also need not be full length, relative to either the primary transcription product or fully processed mRNA. This may be preferred to avoid concurrent production of some plants that are overexpressers. A higher identity in a shorter than full-length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

Another method to decrease expression of a gene (either endogenous or exogenous) is via siRNAs. siRNAs can be applied to a plant and taken up by plant cells; alternatively, siRNAs can be expressed in vivo from an expression cassette. RNAi refers to the introduction of homologous double stranded RNA (dsRNA) to target a specific gene product, resulting in post-transcriptional silencing of that gene. This phenomena was first reported in Caenorhabditis elegans by Guo and Kemphues (Par-1, A gene required for establishing polarity in C. elegans embryos, encodes a putative Ser/Thr kinase that is asymmetrically distributed, 1995, Cell, 81 (4) 611-620) and subsequently Fire et al. (Potent and specific genetic interference by double-stranded RNA in Caenorhabditis elegans, 1998, Nature 391: 806-811) discovered that it is the presence of dsRNA, formed from the annealing of sense and antisense strands present in the in vitro RNA preps, that is responsible for producing the interfering activity.

The present invention contemplates the use of RNA interference (RNAi) to downregulate the expression of lut1 genes. The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial. In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs.

Carthew has reported (Curr. Opin. Cell Biol. 13(2):244-248 (2001) that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

In preferred embodiments, the dsRNA used to initiate RNAi, may be isolated from native source or produced by known means, e.g., transcribed from DNA. The promoters and vectors described in more detail below are suitable for producing dsRNA. RNA is synthesized either in vivo or in vitro. In some embodiments, endogenous RNA polymerase of the cell may mediate transcription in vivo, or cloned RNA polymerase can be used for transcription in vivo or in vitro. In other embodiments, the RNA is provided transcription from a transgene in vivo or an expression construct. In some embodiments, the RNA strands are polyadenylated; in other embodiments, the RNA strands are capable of being translated into a polypeptide by a cell's translational apparatus. In still other embodiments, the RNA is chemically or enzymatically synthesized by manual or automated reactions. In further embodiments, the RNA is synthesized by a cellular RNA polymerase or a bacteriophage RNA polymerase (e.g., T3, T7, SP6). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. In some embodiments, the RNA is dried for storage or dissolved in an aqueous solution. In other embodiments, the solution contains buffers or salts to promote annealing, and/or stabilization of the duplex strands.

In some embodiments, the dsRNA is transcribed from the vectors as two separate stands. In other embodiments, the two strands of DNA used to form the dsRNA may belong to the same or two different duplexes in which they each form with a DNA strand of at least partially complementary sequence. When the dsRNA is thus-produced, the DNA sequence to be transcribed is flanked by two promoters, one controlling the transcription of one of the strands, and the other that of the complementary strand. These two promoters may be identical or different. In some embodiments, a DNA duplex provided at each end with a promoter sequence can directly generate RNAs of defined length, and which can join in pairs to form a dsRNA. See, e.g., U.S. Pat. No. 5,795,715, incorporated herein by reference. RNA duplex formation may be initiated either inside or outside the cell.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases.

There is no upper limit on the length of the dsRNA that can be used. For example, the dsRNA can range from about 21 base pairs (bp) of the gene to the full length of the gene or more. In one embodiment, the dsRNA used in the methods of the present invention is about 1000 bp in length. In another embodiment, the dsRNA is about 500 bp in length. In yet another embodiment, the dsRNA is about 22 bp in length. In some preferred embodiments, the sequences that mediate RNAi are from about 21 to about 23 nucleotides. That is, the isolated RNAs of the present invention mediate degradation of the target RNA (e.g., major sperm protein, chitin synthase, or RNA polymerase II). In preferred embodiments, dsRNAs corresponding to all or a portion of nucleic acids encoding a polypeptide comprising SEQ ID NOs: 1-4, 16-21, 33-39, 49-52 and 56, or nucleic acids corresponding to SEQ ID NOs: 5-9, 22-27, 40-48, 53-55, 57 and 58 are utilized.

The double stranded RNA of the present invention need only be sufficiently similar to natural RNA that it has the ability to mediate RNAi for the target RNA. In one embodiment, the present invention relates to RNA molecules of varying lengths that direct cleavage of specific mRNA to which their sequence corresponds. It is not necessary that there be perfect correspondence of the sequences, but the correspondence must be sufficient to enable the RNA to direct RNAi cleavage of the target mRNA. In a particular embodiment, the RNA molecules of the present invention comprise a 3′ hydroxyl group. In some embodiments, the amount of target RNA (e.g., lut1mRNA) is reduced in the cells of the plant exposed to target specific double stranded RNA as compared to cells of the plant or a control plant that have not been exposed to target specific double stranded RNA.

In still further embodiments, knockouts may be generated by homologous recombination. In some embodiments, knockouts may be generated by heterologous recombination. In some embodiments knockouts may be generated by Agrobacterium transfer-DNA. Generally, plant cells are incubated with a strain of Agrobacterium that contains a targeting vector in which sequences that are homologous to a DNA sequence inside the target locus are flanked by Agrobacterium transfer-DNA (T-DNA) sequences, as previously described (U.S. Pat. No. 5,501,967, herein incorporated by reference) and herein described in Example 1. The term “Agrobacterium” refers to a soil-borne, Gram-negative, rod-shaped phytopathogenic bacterium which causes crown gall. The term “Agrobacterium” includes, but is not limited to, the strains Agrobacterium tumefaciens, (which typically causes crown gall in infected plants), and Agrobacterium rhizogens (which causes hairy root disease in infected host plants). Infection of a plant cell with Agrobacterium generally results in the production of opines (e.g., nopaline, agropine, octopine etc.) by the infected cell. Thus, Agrobacterium strains which cause production of nopaline (e.g., strain GV3101, LBA4301, C58, A208, etc.) are referred to as “nopaline-type” Agrobacteria; Agrobacterium strains which cause production of octopine (e.g., strain LBA4404, Ach5, B6, etc.) are referred to as “octopine-type” Agrobacteria; and Agrobacterium strains which cause production of agropine (e.g., strain EHA105, EHA101, A281, etc.) are referred to as “agropine-type” Agrobacteria.

One of skill in the art knows that homologous recombination may be achieved using targeting vectors that contain sequences that are homologous to any part of the targeted plant gene, whether belonging to the regulatory elements of the gene, or the coding regions of the gene. Homologous recombination may be achieved at any region of a plant gene so long as the nucleic acid sequence of regions flanking the site to be targeted is known.

A. Transgenic Plants, Seeds, and Plant Parts

Plants are transformed with at least one heterologous gene encoding a LUT1 or CYP97A gene, or encoding a sequence designed to decrease LUT1 or CYP97A gene expression, according to any procedure well known or developed in the art. It is contemplated that these heterologous genes, or nucleic acid sequences of the present invention and of interest, are utilized to increase the level of the polypeptide encoded by heterologous genes, or to decrease the level of the protein encoded by endogenous genes. It is contemplated that these heterologous genes, or nucleic acid sequences of the present invention and of interest, are utilized augment and/or increase the level of the protein encoded by endogenous genes. It is also contemplated that these heterologous genes, or nucleic acid sequences of the present invention and of interest, are utilized to provide a polypeptide encoded by heterologous genes. The term “transgenic” when used in reference to a plant or leaf or fruit or seed for example a “transgenic plant,” transgenic leaf,” “transgenic fruit,” “transgenic seed,” or a “transgenic host cell” refers to a plant or leaf or fruit or seed that contains at least one heterologous or foreign gene in one or more of its cells. The term “transgenic plant material” refers broadly to a plant, a plant structure, a plant tissue, a plant seed or a plant cell that contains at least one heterologous gene in one or more of its cells.

1. Plants and Seeds

The methods of the present invention are not limited to any particular plant comprising a heterologous nucleic acid (e.g., plants comprising a heterologous nucleic acid encoding a polypeptide comprising SEQ ID NOs: 1-4, 16-21, 33-39, 49-52 and 56, or nucleic acids corresponding to SEQ ID NOs: 5-9, 22-27, 40-48, 53-55, 57 and 58). Indeed, a variety of plants are contemplated, including but not limited to tomato, sunflowers, rice, corn, barley, wheat, Brassica, Arabidopsis, sunflower, marigolds, and soybean. The term “plant” is used in it broadest sense. It includes, but is not limited to, any species of woody, ornamental or decorative, crop or cereal, fruit or vegetable, fruit plant or vegetable plant, flower or tree, macroalga or microalga, phytoplankton and photosynthetic algae (e.g., green algae Chlamydomonas reinhardtii and diatom Skeletonema costatum). It also refers to a uniclelluar plant (e.g. microalga) and a plurality of plant cells that are largely differentiated into a colony (e.g. volvox) or a structure that is present at any stage of a plant's development. Such structures include, but are not limited to, a fruit, a seed, a shoot, a stem, a leaf, a flower petal, etc. The term “plant tissue” includes differentiated and undifferentiated tissues of plants including those present in roots, shoots, leaves, pollen, seeds and tumors, as well as cells in culture (e.g., single cells, protoplasts, embryos, callus, etc.). In one embodiment, transgenic seeds of the present invention may contain 5× as much β-carotene over wild-type seeds. Plant tissue may be in planta, in organ culture, tissue culture, or cell culture. The term “plant part” as used herein refers to a plant structure or a plant tissue. In some embodiments of the present invention transgenic plants are crop plants. The term “crop” or “crop plant” is used in its broadest sense. The term includes, but is not limited to, any species of plant or alga edible by humans or used as a feed for animals or fish or marine animals, or consumed by humans, or used by humans (natural pesticides), or viewed by humans (flowers) or any plant or alga used in industry or commerce or education.

2. Vectors

The methods of the present invention contemplate the use of at least one heterologous gene encoding a LUT1 gene, or a CYP97A gene, or encoding a sequence designed to decrease or increase, LUT1, or CYP97A gene expression, as described previously (e.g., vectors encoding a nucleic acid encoding a polypeptide comprising SEQ ID NOs: 1-4, 16-21, 33-39, 49-52 and 56, or nucleic acids corresponding to SEQ ID NOs: 5-9, 22-27, 40-48, 53-55, 57 and 58). Heterologous genes include but are not limited to naturally occurring coding sequences, as well variants encoding mutants, variants, truncated proteins, and fusion proteins, as described above.

Heterologous genes intended for expression in plants are first assembled in expression cassettes comprising a promoter. Methods which are well known to or developed by those skilled in the art may be used to construct expression vectors containing a heterologous gene and appropriate transcriptional and translational control elements. These methods include in vitro recombinant DNA techniques, synthetic techniques, and in vivo genetic recombination. Exemplary techniques are widely described in the art (see e.g., Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y., herein incorporated by reference).

In general, these vectors comprise a nucleic acid sequence encoding a lut1 gene, or a CYP97A gene, or encoding a sequence designed to decrease lut1 gene, or CYP97A gene expression, (as described above) operably linked to a promoter and other regulatory sequences (e.g., enhancers, polyadenylation signals, etc.) required for expression in a plant.

Promoters include but are not limited to constitutive promoters, tissue-, organ-, and developmentally-specific promoters, and inducible promoters. Examples of promoters include but are not limited to: constitutive promoter 35S of cauliflower mosaic virus; a wound-inducible promoter from tomato, leucine amino peptidase (“LAP,” Chao et al., Plant Physiol 120: 979-992 (1999), herein incorporated by reference); a chemically-inducible promoter from tobacco, Pathogenesis-Related 1 (PR1) (induced by salicylic acid and BTH (benzothiadiazole-7-carbothioic acid S-methyl ester)); a tomato proteinase inhibitor II promoter (PIN2) or LAP promoter (both inducible with methyl jasmonate); a heat shock promoter (U.S. Pat. No. 5,187,267, herein incorporated by reference); a tetracycline-inducible promoter (U.S. Pat. No. 5,057,422, herein incorporated by reference); and seed-specific promoters, such as those for seed storage proteins (e.g., phaseolin, napin, oleosin, and a promoter for soybean beta conglycin (Beachy et al., EMBO J. 4: 3047-3053 (1985), herein incorporated by reference). All references cited herein are incorporated in their entirety.

The expression cassettes may further comprise any sequences required for expression of mRNA. Such sequences include, but are not limited to transcription terminators, enhancers such as introns, viral sequences, and sequences intended for the targeting of the gene product to specific organelles and cell compartments.

A variety of transcriptional terminators are available for use in expression of sequences using the promoters of the present invention. Transcriptional terminators are responsible for the termination of transcription beyond the transcript and its correct polyadenylation. Appropriate transcriptional terminators and those which are known to function in plants include, but are not limited to, the CaMV 35S terminator, the tml terminator, the pea rbcS E9 terminator, and the nopaline and octopine synthase terminator (See e.g., Odell et al., Nature 313:810 (1985); Rosenberg et al., Gene, 56:125 (1987); Guerineau et al., Mol. Gen. Genet., 262:141 (1991); Proudfoot, Cell, 64:671 (1991); Sanfacon et al., Genes Dev., 5:141; Mogen et al., Plant Cell, 2:1261 (1990); Munroe et al., Gene, 91:151 (1990); Ballas et al., Nucleic Acids Res. 17:7891 (1989); Joshi et al., Nucleic Acid Res., 15:9627 (1987), all of which are incorporated herein by reference).

In addition, in some embodiments, constructs for expression of the gene of interest include one or more of sequences found to enhance gene expression from within the transcriptional unit. These sequences can be used in conjunction with the nucleic acid sequence of interest to increase expression in plants. Various intron sequences have been shown to enhance expression, particularly in monocotyledonous cells. For example, the introns of the maize Adh1 gene have been found to significantly enhance the expression of the wild-type gene under its cognate promoter when introduced into maize cells (Callis et al., Genes Develop. 1: 1183 (1987), herein incorporated by reference). Intron sequences have been routinely incorporated into plant transformation vectors, typically within the non-translated leader.

In some embodiments of the present invention, the construct for expression of the nucleic acid sequence of interest also includes a regulator such as a nuclear localization signal (Kalderon et al., Cell 39:499 (1984); Lassner et al., Plant Molecular Biology 17:229 (1991)), a plant translational consensus sequence (Joshi, Nucleic Acids Research 15:6643 (1987)), an intron (Luehrsen and Walbot, Mol.Gen. Genet. 225:81 (1991)), and the like, operably linked to the nucleic acid sequence encoding a LUT1 gene.

In preparing the construct comprising the nucleic acid sequence encoding a LUT1 gene, or encoding a sequence designed to decrease LUT1 gene expression, various DNA fragments can be manipulated, so as to provide for the DNA sequences in the desired orientation (e.g., sense or antisense) orientation and, as appropriate, in the desired reading frame. For example, adapters or linkers can be employed to join the DNA fragments or other manipulations can be used to provide for convenient restriction sites, removal of superfluous DNA, removal of restriction sites, or the like. For this purpose, in vitro mutagenesis, primer repair, restriction, annealing, resection, ligation, or the like is preferably employed, where insertions, deletions or substitutions (e.g., transitions and transversions) are involved.

Numerous transformation vectors are available for plant transformation. The selection of a vector for use will depend upon the preferred transformation technique and the target species for transformation. For certain target species, different antibiotic or herbicide selection markers are preferred. Selection markers used routinely in transformation include the nptII gene which confers resistance to kanamycin and related antibiotics (Messing and Vierra, Gene 19: 259 (1982); Bevan et al., Nature 304:184 (1983), all of which are incorporated herein by reference), the bar gene which confers resistance to the herbicide phosphinothricin (White et al., Nucl Acids Res. 18:1062 (1990); Spencer et al., Theor. Appl. Genet. 79: 625 (1990), all of which are incorporated herein by reference), the hph gene which confers resistance to the antibiotic hygromycin (Blochlinger and Diggelmann, Mol. Cell. Biol. 4:2929 (1984, incorporated herein by reference)), and the dhfr gene, which confers resistance to methotrexate (Bourouis et al., EMBO J., 2:1099 (1983), herein incorporated by reference).

In some preferred embodiments, the (Ti(T-DNA)plasmid) vector is adapted for use in an Agrobacterium mediated transfection process (See e.g., U.S. Pat. Nos. 5,981,839; 6,051,757; 5,981,840; 5,824,877; and 4,940,838; all of which are herein incorporated by reference). Construction of recombinant Ti and Ri plasmids in general follows methods typically used with the more common vectors, such as pBR322. Additional use can be made of accessory genetic elements sometimes found with the native plasmids and sometimes constructed from foreign sequences. These may include but are not limited to structural genes for antibiotic resistance as selection genes.

There are two systems of recombinant Ti and Ri plasmid vector systems now in use. The first system is called the “cointegrate” system. In this system, the shuttle vector containing the gene of interest is inserted by genetic recombination into a non-oncogenic Ti plasmid that contains both the cis-acting and trans-acting elements required for plant transformation as, for example, in the pMLJ1 shuttle vector and the non-oncogenic Ti plasmid pGV3850. The use of T-DNA as a flanking region in a construct for integration into a Ti- or Ri-plasmid has been described in EPO No. 116,718 and PCT Application Nos. WO 84/02913, 02919 and 02920 all of which are herein incorporated by reference). See also Herrera-Estrella, Nature 303:209-213 (1983); Fraley et al., Proc. Natl. Acad. Sci, USA 80:4803-4807 (1983); Horsch et al., Science 223:496-498 (1984); and DeBlock et al., EMBO J. 3:1681-1689 (1984), all of which are herein incorporated by reference). The second system is called the “binary” system in which two plasmids are used; the gene of interest is inserted into a shuttle vector containing the cis-acting elements required for plant transformation. The other necessary functions are provided in trans by the non-oncogenic Ti plasmid as exemplified by the pBIN19 shuttle vector and the non-oncogenic Ti plasmid PAL4404. Some of these vectors are commercially available. In other embodiments of the invention, the nucleic acid sequence of interest is targeted to a particular locus on the plant genome. Site-directed integration of the nucleic acid sequence of interest into the plant cell genome may be achieved by, for example, homologous recombination using Agrobacterium-derived sequences. Generally, plant cells are incubated with a strain of Agrobacterium which contains a targeting vector in which sequences that are homologous to a DNA sequence inside the target locus are flanked by Agrobacterium transfer-DNA (T-DNA) sequences, as previously described (U.S. Pat. No. 5,501,967 herein incorporated by reference). One of skill in the art knows that homologous recombination may be achieved using targeting vectors that contain sequences that are homologous to any part of the targeted plant gene, whether belonging to the regulatory elements of the gene, or the coding regions of the gene. Homologous recombination may be achieved at any region of a plant gene so long as the nucleic acid sequence of regions flanking the site to be targeted is known. Agrobacterium tumefaciens is a common soil bacterium that causes crown gall disease by transferring some of its DNA to the plant host. The transferred DNA (T-DNA) is stably integrated into the plant genome, where its expression leads to the synthesis of plant hormones and thus to the tumorous growth of the cells. A putative macromolecular complex forms in the process of T-DNA transfer out of the bacterial cell into the plant cell.

In yet other embodiments, the nucleic acids of the present invention is utilized to construct vectors derived from plant (+) RNA viruses (e.g., brome mosaic virus, tobacco mosaic virus, alfalfa mosaic virus, cucumber mosaic virus, tomato mosaic virus, and combinations and hybrids thereof). Generally, the inserted LUT1 polynucleotide can be expressed from these vectors as a fusion protein (e.g., coat protein fusion protein) or from its own subgenomic promoter or other promoter. Methods for the construction and use of such viruses are described in U.S. Pat. Nos. 5,846,795; 5,500,360; 5,173,410; 5,965,794; 5,977,438; and 5,866,785, all of which are incorporated herein by reference.

In some embodiments of the present invention, where the nucleic acid sequence of interest is introduced directly into a plant. One vector useful for direct gene transfer techniques in combination with selection by the herbicide Basta (or phosphinothricin) is a modified version of the plasmid pCIB246, with a CaMV 35S promoter in operational fusion to the E. coli GUS gene and the CaMV 35S transcriptional terminator (WO 93/07278).

3. Transformation Techniques

Once a nucleic acid sequence encoding a LUT1 gene is operatively linked to an appropriate promoter and inserted into a suitable vector for the particular transformation technique utilized (e.g., one of the vectors described above), the recombinant DNA described above can be introduced into the plant cell in a number of art-recognized ways. Those skilled in the art will appreciate that the choice of method might depend on the type of plant targeted for transformation. In some embodiments, the vector is maintained episomally. In other embodiments, the vector is integrated into the genome.

In some embodiments, direct transformation in the plastid genome is used to introduce the vector into the plant cell (See e.g., U.S. Pat. Nos. 5,451,513; 5,545,817; 5,545,818; PCT application WO 95/16783 all of which are incorporated herein by reference). The basic technique for chloroplast transformation involves introducing regions of cloned plastid DNA flanking a selectable marker together with the nucleic acid encoding the RNA sequences of interest into a suitable target tissue (e.g., using biolistics or protoplast transformation with calcium chloride or PEG). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., PNAS, 87:8526 (1990); Staub and Maliga, Plant Cell, 4:39 (1992), all of which are incorporated herein by reference). The presence of cloning sites between these markers allowed creation of a plastid targeting vector introduction of foreign DNA molecules (Staub and Maliga, EMBO J., 12:601 (1993)). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab and Maliga, PNAS, 90:913 (1993)). Other selectable markers useful for plastid transformation are known in the art and encompassed within the scope of the present invention. Plants homoplasmic for plastid genomes containing the two nucleic acid sequences separated by a promoter of the present invention are obtained, and are preferentially capable of high expression of the RNAs encoded by the DNA molecule.

In other embodiments, vectors useful in the practice of the present invention are microinjected directly into plant cells by use of micropipettes to mechanically transfer the recombinant DNA (Crossway, Mol. Gen. Genet, 202:179 (1985)). In still other embodiments, the vector is transferred into the plant cell by using polyethylene glycol (Krens et al., Nature, 296:72 (1982); Crossway et al., BioTechniques, 4:320 (1986)); fusion of protoplasts with other entities, either minicells, cells, lysosomes or other fusible lipid-surfaced bodies (Fraley et al., Proc. Natl. Acad. Sci., USA, 79:1859 (1982)); protoplast transformation (EP 0 292 435); direct gene transfer (Paszkowski et al., EMBO J., 3:2717 (1984); Hayashimoto et al., Plant Physiol. 93:857 (1990)).

In still further embodiments, the vector may also be introduced into the plant cells by electroporation. (Fromm, et al., Pro. Natl Acad. Sci. USA 82:5824, 1985; Riggs et al., Proc. Natl. Acad. Sci. USA 83:5602 (1986)). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

In yet other embodiments, the vector is introduced through ballistic particle acceleration using devices (e.g., available from Agracetus, Inc., Madison, Wis. and Dupont, Inc., Wilmington, Del.). (See e.g., U.S. Pat. No. 4,945,050; and McCabe et al., Biotechnology 6:923 (1988)). See also, Weissinger et al., Annual Rev. Genet. 22:421 (1988); Sanford et al., Particulate Science and Technology, 5:27 (1987) (onion); Svab et al., Proc. Natl. Acad. Sci. USA, 87:8526 (1990) (tobacco chloroplast); Christou et al., Plant Physiol., 87:671 (1988) (soybean); McCabe et al., Bio/Technology 6:923 (1988) (soybean); Klein et al., Proc. Natl. Acad. Sci. USA, 85:4305 (1988) (maize); Klein et al., Bio/Technology, 6:559 (1988) (maize); Klein et al., Plant Physiol., 91:4404 (1988) (maize); Fromm et al., Bio/Technology, 8:833 (1990); and Gordon-Kamm et al., Plant Cell, 2:603 (1990) (maize); Koziel et al., Biotechnology, 11:194 (1993) (maize); Hill et al., Euphytica, 85:119 (1995) and Koziel et al., Annals of the New York Academy of Sciences 792:164 (1996); Shimamoto et al., Nature 338: 274 (1989) (rice); Christou et al., Biotechnology, 9:957 (1991) (rice); Datta et al., Bio/Technology 8:736 (1990) (rice); European Application EP 0 332 581 (orchardgrass and other Pooideae); Vasil et al., Biotechnology, 11: 1553 (1993) (wheat); Weeks et al., Plant Physiol., 102: 1077 (1993) (wheat); Wan et al., Plant Physiol. 104: 37 (1994) (barley); Jahne et al., Theor. Appl. Genet. 89:525 (1994) (barley); Knudsen and Muller, Planta, 185:330 (1991) (barley); Umbeck et al., Bio/Technology 5: 263 (1987) (cotton); Casas et al., Proc. Natl. Acad. Sci. USA 90:11212 (1993) (sorghum); Somers et al., Bio/Technology 10:1589 (1992) (oat); Torbert et al., Plant Cell Reports, 14:635 (1995) (oat); Weeks et al., Plant Physiol., 102:1077 (1993) (wheat); Chang et al., WO 94/13822 (wheat) and Nehra et al., The Plant Journal, 5:285 (1994) (wheat) herein incorporated by reference.

In addition to direct transformation, in some embodiments, the vectors comprising a nucleic acid sequence encoding a LUT1 gene are transferred using Agrobacterium-mediated transformation (Hinchee et al., Biotechnology, 6:915 (1988); Ishida et al., Nature Biotechnology 14:745 (1996), all of which are herein incorporated by reference). Agrobacterium is a representative genus of the gram-negative family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines are a convenient source of control elements for chimeric expression cassettes. Heterologous genetic sequences (e.g., nucleic acid sequences operatively linked to a promoter of the present invention), can be introduced into appropriate plant cells, by means of the Ti plasmid of Agrobacterium tumefaciens. The Ti plasmid is transmitted to plant cells on infection by Agrobacterium tumefaciens, and is stably integrated into the plant genome (Schell, Science, 237: 1176 (1987)). Species which are susceptible infection by Agrobacterium may be transformed in vitro.

4. Regeneration

After selecting for transformed plant material that can express a heterologous gene encoding a LUT1 gene, or a CYP97A gene or variant thereof, whole plants are regenerated. Plant regeneration from cultured protoplasts is described in Evans et al., Handbook of Plant Cell Cultures, Vol. 1: (MacMillan Publishing Co. New York, 1983); and Vasil I. R. (ed.), Cell Culture and Somatic Cell Genetics of Plants, Acad. Press, Orlando, Vol. I, 1984, and Vol. III, 1986, herein incorporated by reference. It is known that many plants can be regenerated from cultured cells or tissues, including but not limited to all major species of sugarcane, sugar beet, cotton, fruit and other trees, legumes and vegetables, and monocots (e.g., the plants described above). Means for regeneration vary from species to species of plants, but generally a suspension of transformed protoplasts containing copies of the heterologous gene is first provided. Callus tissue is formed and shoots may be induced from callus and subsequently rooted.

Alternatively, embryo formation can be induced from the protoplast suspension. These embryos germinate and form mature plants. The culture media will generally contain various amino acids and hormones, such as auxin and cytokinins. Shoots and roots normally develop simultaneously. Efficient regeneration will depend on the medium, on the genotype, and on the history of the culture. The reproducibility of regeneration depends on the control of these variables.

5. Generation of Transgenic Lines

Transgenic lines are established from transgenic plants by tissue culture propagation. The presence of nucleic acid sequences encoding an exogenous LUT1 gene, or a CYP97A gene or mutants or variants thereof may be transferred to related varieties by traditional plant breeding techniques. Examples of transgenic lines are described herein and in Example 1.

These transgenic lines are then utilized for evaluation of carotenoid production, carotenoid ratios, phenotype, color, pathogen resistance and other agronomic traits.

B. Evaluation of Carotenoid Production

The transgenic plants and lines are tested for the effects of the transgene on carotenoid phenotype. The parameters evaluated for carotenoids are compared to those in control untransformed plants and lines. Parameters evaluated include rates of carotenoid production, effects of light, heat, cold; effects on altering steady-state ratios and effects on carotenoid production. Rates of carotenoid production can be expressed as a unit of time, or in a particular tissue or as a developmental state; for example, carotenoid production Arabidopsis can be measured in leaves and seeds. These tests are conducted both in the greenhouse and in the field. The terms “altered carotenoid ratios” and “altering carotenoid ratios” refers to any changes in carotenoid production. An example of such changes are shown in FIG. 13.

The present invention also provides any of the isolated nucleic acid sequences described above operably linked to a promoter. In some embodiments, the promoter is a heterologous promoter. In other embodiments, the promoter is a plant promoter. The present invention also provides a vector comprising any of the nucleic acid sequences described above. In some embodiments, the vector is a cloning vector; in other embodiments, the vector is an expression vector. In some further embodiments, the nucleic acid sequence in the vector is linked to a promoter. In some further embodiments, the promoter is a heterologous promoter. In other further embodiments, the promoter is a plant promoter.

The present invention also provides a transgenic host cell comprising any of the nucleic acid sequences of the present invention described above, wherein the nucleic acid sequence is heterologous to the host cell. In some embodiments, the nucleic acid sequence is operably linked to any of the promoters described above. In other embodiments, the nucleic acid is present in any of the vectors described above.

The present invention also provides a transgenic organism comprising any of the nucleic acid sequences of the present invention described above, wherein the nucleic acid sequence is heterologous to the organism. In some embodiments, the nucleic acid sequence is operably linked to any of the promoters described above. In other embodiments, the nucleic acid is present in any of the vectors described above.

The present invention also provides a transgenic plant, a transgenic plant part, a transgenic plant cell, or a transgenic plant seed, comprising any of the nucleic acid sequences of the present invention described above, wherein the nucleic acid sequence is heterologous to the transgenic plant, a transgenic plant part, a transgenic plant cell, or a transgenic plant seed. In some embodiments, the nucleic acid sequence is operably linked to any of the promoters described above. In other embodiments, the nucleic acid is present in any of the vectors described above.

The present invention also provides a method for producing a LUT1 and/or a CYP97A polypeptide, comprising culturing a transgenic host cell comprising a heterologous nucleic acid sequence, wherein the heterologous nucleic acid sequence is any of the nucleic acid sequences of the present invention described above which encode a LUT1 and/or a CYP97A polypeptide or variant thereof, under conditions sufficient for expression of the encoded LUT1 and/or a CYP97A polypeptide, and producing the LUT1 and/or a CYP97A polypeptide in the transgenic host cell. In some embodiments, the nucleic acid sequence is operably linked to any of the promoters described above. In other embodiments, the nucleic acid is present in any of the vectors described above. The present invention also provides a method for producing a LUT1 and/or a CYP97A polypeptide, comprising growing a transgenic host cell comprising a heterologous nucleic acid sequence, wherein the heterologous nucleic acid sequence is any of the nucleic acid sequences of the present invention described above encoding a LUT1 and/or a CYP97A polypeptide or a variant thereof, under conditions sufficient for expression of the encoded LUT1 and/or a CYP97A polypeptide, and producing the LUT1 and/or a CYP97A polypeptide in the transgenic host cell.

The present invention also provides a method for altering the phenotype of a plant, comprising providing an expression vector comprising any of the nucleic acid sequences of the present invention described above, and plant tissue, and transfecting the plant tissue with the vector under conditions such that a plant is obtained from the transfected tissue and the nucleic acid sequence is expressed in the plant and the phenotype of the plant is altered. In some embodiments, the nucleic acid sequence encodes a LUT1 and/or a CYP97A polypeptide or variant thereof. In other embodiments, the nucleic sequence encodes a nucleic acid product which interferes with the expression of a nucleic acid sequence encoding a LUT1 and/or a CYP97A polypeptide or variant thereof, wherein the interference is based upon the coding sequence of the LUT1 and/or a CYP97A protein or variant thereof. In some embodiments, the nucleic acid sequence is operably linked to any of the promoters described above. In other embodiments, the nucleic acid is present in any of the vectors described above.

The present invention also provides a method for altering the phenotype of a plant, comprising growing a transgenic plant comprising an expression vector comprising any of the nucleic acid sequences of the present invention described above under conditions such that the nucleic acid sequence is expressed and the phenotype of the plant is altered. In some embodiments, the nucleic acid sequence encodes a LUT1 and/or a CYP97A polypeptide or variant thereof. In other embodiments, the nucleic sequence encodes a nucleic acid product which interferes with the expression of a nucleic acid sequence encoding a LUT1 and/or a CYP97A polypeptide or variant thereof, wherein the interference is based upon the coding sequence of the LUT1 and/or a CYP97A protein or variant thereof. In some embodiments, the nucleic acid sequence is operably linked to any of the promoters described above. In other embodiments, the nucleic acid is present in any of the vectors described above.

### Experimental

The following examples serve to illustrate certain embodiments and aspects of the present invention and are not to be construed as liming the scope thereof. In the experimental disclosures which follow, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); pg (picograms); L or l (liters); ml (milliliters); μl (microliters); cm (centimeters); mm (millimeters); μm (micrometers); nm (nanometers); ° C. (degrees Centigrade).

### EXAMPLE 1

### Materials and Methods

The following is a description of exemplary materials and methods that were used in subsequent Examples.

Mutant Screening Service:

The University of Wisconsin Arabidopsis T-DNA knockout facility provided mutant screening service.

Positional Cloning of LUT1. Homozygous lut1-1 (ecotype Columbia) was crossed to wild type Landsberg erecta. F₂ progeny homozygous for the lut1 mutation were identified by a thin-layer chromatography (TLC) screening method. Briefly, carotenoid samples were extracted as described (Tian, et al. Plant Mol. Biol. 47, 379-388 (2001), herein incorporated by reference) resuspended in ethyl acetate, spotted on a silica TLC plate (J. T. Baker, Phillipsburg, N.J.), and developed in 90:10 (v:v) hexane: isopropanol. F₂ plants homozygous for lut1 contain a characteristic extra yellow band due to accumulation of zeinoxanthin.

Genomic DNA from homozygous lut1 F₂ plants was isolated using the DNAzol reagent following the manufacturer's instructions (Invitrogen, Carlsbad, Calif.). PCR reactions were performed with 1 μl of genomic DNA in a 20 μl reaction mixture. The PCR program was 94° C. for 3 min, 60 cycles of 94° C. for 15 s, 50° C.-60° C. (the annealing temperature was optimized for each specific pair of primers) for 30 s, 72° C. for 30 s, and finally 72° C. for 10 min. A portion of the PCR product was then separated on a 3% agarose gel. lut1 had been previously mapped to 67±3 cM on chromosome 3 (Tian, et al. Plant Mol. Biol. 47, 379-388 (2001). Additional Simple Sequence Length Polymorphism (SSLP) markers for fine mapping in this interval were designed based on the insertions/deletions (INDELs) information obtained from the Monsanto website: http://www.arabidopsis.org/Cereon/.

Cosmid Screening and Complementation of lut1. An Arabidopsis cosmid library (13) was screened and cosmids carrying the At3g53130 gene were identified. For complementation of the lut1 mutation, a 4.2 kb restriction fragment containing the At3g53130 gene was subcloned into the pMLBART vector (Gleave, Plant Mol. Biol. 20, 1203-1207 (1992). Homozygous lut1 plants were transformed with Agrobacterium tumefaciens strain GV3101 containing pMLBART-At3g53130 using the Floral Dip method (Clough and Bent, Plant J. 16, 735-743 (1998), herein incorporated by reference). BASTA-resistant T₁ transformants were selected and the carotenoid composition of leaf tissue was analyzed by HPLC (Tian, et al. Plant Mol. Biol. 47, 379-388 (2001), herein incorporated by reference).

Isolation of T-DNA Knockout Mutants in At3g53130 and Generation of a Carotenoid Hydroxylase Triple Knockout Mutant Line. At3g53130 specific primers (forward, 5′-CTTCCTCTTCTTACTCTTCTCTCTTCACT-3′; reverse, 5′-AAGAACGATGGATGTTATAGACTGAAATC-3′) were sent to the University of Wisconsin Arabidopsis T-DNA knockout facility to identify knockout mutants of the LUT1 gene. A single knockout line, designated lut1-3, was identified and isolated as described (http://www.biotech.wisc.edu/Arabidopsis/). In order to generate a hydroxylase triple knockout mutant line, homozygous lut1-3 and b1 b2 plants were crossed. Putative lut1-3 b1 b2 triple mutants were identified from the segregating F₂ population by HPLC and their genotypes confirmed by PCR as previously described (Tian, et al. Plant Cell 15, 1320-1332 (2003), herein incorporated by reference).

TaqMan Real-Time PCR Assay. LUT1 mRNA levels were quantified by TaqMan real-time PCR using elongation factor EF1α mRNA levels for normalization (Tian, et al. Plant Cell 15, 1320-1332 (2003), herein incorporated by reference]. The LUT1 TaqMan probe and primers are: 5′-CCGTCTCGCTGCTGGTCCTCG-3′ (TaqMan probe), 5′-GGATGAATGAGTACGGACCCAT-3′ (forward primer), and 5′-GGGTCGCTCACAATTACGAAA-3′ (reverse primer). The relative quantity of the transcripts was calculated using the comparative C_{T} method [Livak, PE applied Biosystems. User Bulletin 2, 11-15 (1997), herein incorporated by reference].

Phylogenetic Analysis of LUT1 Homologs. Full-length protein sequences of putative LUT1 homologs from Arabidopsis thaliana, Glycine Max, Oryza sativa, and Pisum sativum were obtained from GenBank: CYP97A3 (AAL08302), CYP97B1 (CAA89260), CYP97B2 (AAB94586), CYP97B3 (CAB10290), CYP97C1 (AAM13903), CYP97C2 (AAK20054) and CYP86A8 (CAC47665). Rice CYP97A4 and CYP97B4 sequences were obtained from the cytochrome P450 website (http://drnelson.utmem.edu/CytochromeP450.html).

Additional plant LUT1 homologs were retrieved from The Institute of Genome Research (TIGR) Unique Gene Indices: TC76166 (Hordeum vulgare), TC163981 (Glycine Max), and TC69886 (Hordeum vulgare). The coding sequences of each were extracted, assembled, and corrected by the ESTscan program (http://tigrblast.tigr.org/tgi/). Chlamydomonas CYP97A3 homolog (Scaffold1399) was obtained from the DOE Joint Genome Institute (JGI) database (http://genome.jgi-psf.org/chlrel/chlrel.home.html). The term “scaffold” refers to a result of connecting contigs by linking information from paired-end reads from plasmids, paired-end reads from BACs, known messenger RNAs or other sources. The contigs in a scaffold are ordered and oriented with respect to one another and sometimes refered to as s supercontig. The term “supercontig” refers to a contig formed when an association can be made between two contigs that have no sequence overlap. This commonly occurs using information obtained from paired plasmid ends. For example, when both ends of a BAC clone are sequenced and it can be inferred that these two sequences are approximately 150-200 Kb apart (based on the average size of a BAC), then further if the sequence from one end is found in a particular sequence contig, and the sequence from the other end is found in a different sequence contig, the two sequence contigs are said to be linked. Truncated LUT1 homologs from Zea mays, lettuce, and cotton are also present in the databases but were not used for phylogenetic analysis because full-length assemblies were not possible.

The deduced amino acid sequences of LUT1 homologs were aligned using the ClustalX algorithm (Thompson, et al. Nucleic Acids Res. 24, 4876-4882 (1997), herein incorporated by reference). A neighbor-joining (Saitou and Nei, Mol. Biol. Evol. 4, 406-425 (1987), herein incorporated by reference) tree was constructed based on the sequence alignment and further tested with 500 bootstrap resamplings using the computer program MEGA2 (version 2.1) (Kumar, et al. Bioinformatics 17, 1244-1245 (2001), herein incorporated by reference). Poisson-correction distance was used with 340 amino acids after removing gaps.

### EXAMPLE 2

### Fine Mapping of the LUT1 Locus

This example describes the identification, cloning, and characterization of the lut1 gene.

The LUT1 locus has previously been mapped to the bottom arm of chromosome 3 at 67±3 cM (Tian, et al. Plant Mol. Biol. 47, 379-388 (2001), herein incorporated by reference). For fine mapping of the locus, 530 plants homozygous for the lut1 mutation were identified from approximately 2,000 plants in a segregating F₂ mapping population. Using SSLP markers, LUT1 was initially localized to an interval spanning two BAC clones (F8J2 and T4D2) and was further delineated to a 100 kb interval containing 30 predicted proteins (FIG. 2A). As with all other carotenoid biosynthetic enzymes, the LUT1 gene product is predicted to be chloroplast-targeted and within the 100 kb interval containing LUT1, six proteins were predicted as being chloroplast-targeted by the TargetP prediction software (http://www.cbs.dtu.dk/services/TargetP). One of these chloroplast-targeted proteins, At3g53130, is a member of the cytochrome P450 monooxygenase family (CYP97C1). Cytochrome P450 monooxygenases are heme-binding proteins that insert a single oxygen atom into substrates, e.g. hydroxylation reactions, and therefore At3g53130 was considered to be a strong candidate for LUT1.

### EXAMPLE 3

### Mutant Complementation, Characterization, and the Identification of LUT1

The identity of At3g53130 as LUT1 was initially demonstrated by molecular complementation analysis. Homozygous lut1-1 mutants were transformed with a 4.2 kb genomic DNA fragment from wild type Columbia (the background of lut1) containing the At3g53130 coding region, 1.0 kb upstream of the start codon, and 0.7 kb downstream of the stop codon. Eight independent transformants were selected and all showed a wild type lutein level when analyzed by HPLC (FIG. 3D). These data indicate that At3g53130 genomic DNA can complement the lut1 mutation.

To determine the molecular basis of the lut1 mutations, we sequenced both original EMS-derived lut1 alleles (Pogson, et al. Plant Cell 8, 1627-1639, (1996), herein incorporated by reference). The lut1-1 allele contains a G to A mutation at the highly conserved exon/intron splice junction (5′ AG/GT, the mutated G is in bold) that would cause an error in RNA splicing and lead to production of a mistranslated protein (FIG. 2B). The coding region of the lut1-2 allele was fully sequenced but no mutations were identified. However, a rearrangement in the upstream region of the lut1-2 allele was identified by Southern blot analysis but was not characterized further (data not shown). A third lut1 allele, lut1-3, was identified by screening a T-DNA knockout population using At3g53130-specific primers. Lut1-3 contains a T-DNA insertion in the sixth intron of the LUT1 gene (FIG. 2B).

In order to compare the impact of different lut1 alleles on carotenoid composition, total carotenoids were extracted from four-week old wild type, lut1-1, lut1-2 (data not shown), and lut1-3 plants and separated by HPLC (FIGS. 3A-C). Lut1-1 and lut1-2 accumulated the monohydroxy biosynthetic intermediate zeinoxanthin and contained 8% of wild type lutein, consistent with prior report (Pogson, et al. Plant Cell 8, 1627-1639, (1996). In contrast, though lut1-3 also accumulated zeinoxanthin it lacked lutein (FIG. 3C), indicating that ε-ring hydroxylation function is eliminated by disruption of the At3g53130 gene. The lut1-3 phenotype also indicates that redundant ε-ring hydroxylation activities are not present in leaves and that the previously reported EMS-mutagenized lut1-1 and lut1-2 alleles are indeed leaky for ε-ring hydroxylation activity (FIG. 3B; 11). Taken together, the complementation of the lut1-1 mutation with a wild type At3g53130 gene, the point mutation at a conserved splice site in the lut1-1 allele, and the phenotype of the At3g53130 T-DNA knockout mutant conclusively demonstrate that At3g53130 is the LUT1 locus.

### EXAMPLE 4

### LUT1 Encodes a Chloroplast-targeted Cytochrome P450 with a Single Transmembrane Domain

The deduced amino acid sequence of LUT1 contains several features characteristic of cytochrome P450 enzymes (FIG. 2C). Cytochrome P450 monooxygenases contain a consensus sequence of (A/G)GX(D/E)T(T/S) that forms a binding pocket for molecular oxygen with the invariant Thr residue playing a critical role in oxygen binding in both prokaryotic and eukaryotic cytochrome P450s (Chapple, Annu. Rev. Plant Physiol. Plant Mol. Biol. 49, 311-343 (1998), herein incorporated by reference). In the deduced LUT1 protein sequence, this oxygen-binding pocket is highly conserved (single underlined amino acids in FIG. 2C). The conserved sequence around the heme-binding cysteine residue for cytochrome P450 type enzymes is FXXGXXXCXG, and is also present in LUT1 (double underlined amino acids in FIG. 2C).

The chloroplast transit peptide prediction software ChloroP v 1.1 (http://www.cbs.dtu.dk/services/ChloroP/) predicts an N-terminal transit peptide in LUT1 that is cleaved between Arg-36 and Ser-37 (FIG. 2C). The predicted chloroplast localization for LUT1 is consistent with the subcellular localization of carotenoid biosynthesis in higher plants (Cunningham and Gantt, Annu. Rev. Plant Physiol. Plant Mol. Biol. 49, 557-583 (1998), herein incorporated by reference) but is uncommon for a plant cytochrome P450. Out of the 272 predicted cytochrome P450s in the Arabidopsis genome, only nine, including LUT 1, are predicted to be chloroplast-targeted (Schuler and Werck-Reichhart, Annu. Rev. Plant Biol. 54, 629-667 (2003), herein incorporated by reference). LUT1 also contains a single predicted transmembrane domain (shaded box, FIG. 2C), which contrasts with the four transmembrane domains predicted for the non-heme di-iron β-hydroxylases (Cunningham and Gantt, Annu. Rev. Plant Physiol. Plant Mol. Biol. 49, 557-583 (1998), herein incorporated by reference). Initial attempts to express and assay LUT1 protein in yeast were unsuccessful.

### EXAMPLE 5

### LUT1 Gene Expression and in vivo Activity in the β-Hydroxylase Deficient Backgrounds

Characterization of previously isolated T-DNA knockouts in the two Arabidopsis β-hydroxylase genes suggested that β- and ε-hydroxylases have overlapping functions in vivo (Tian, et al. Plant Cell 15, 1320-1332 (2003). In order to investigate whether ε-hydroxylase expression is affected in the various carotenoid hydroxylase mutant backgrounds, steady state LUT1 mRNA levels were quantified by real-time PCR (FIG. 4). The LUT1 TaqMan probe hybridizes 336 bp downstream from the start codon. LUT1 mRNA levels are not significantly different from wild type in the β-hydroxylase single mutants (b1 and b2), but are significantly increased in the β-hydroxylase double mutant b1 b2 (FIG. 4). LUT1 mRNA levels in lut1-2 alone and in combination with various β-hydroxylase mutant loci (i.e. lut1-2 b1, lut1-2 b2, and lut1-2 b1 b2) are similar and reduced to 2% of wild type levels, consistent with the rearrangement of the upstream region in lut1-2 negatively impacting LUT1 transcription. The steady-state levels of modified LUT1 transcript in lut1-1 and lut1-3 are similar to wild type transcript levels suggesting that although LUT1 activity is negatively impacted in each mutant, LUT1 transcription is not.

The phenotype of the previously isolated lut1-2 b1 b2 mutant was not conclusive due to the leaky nature of the EMS-derived lut1 -2 allele. Cloning of LUT1 and isolation of the LUT1 knockout mutant, lut1-3, allow for the complete elimination of LUT1 activity in vivo. Lut1-3 was crossed to b1 b2 and homozygous lut1-3 b1 b2 mutants were isolated. There was no lutein production in the lut1-3 b1 b2 triple mutant (data not shown), consistent with the lut1-3 single mutant phenotype (FIG. 3C). The total moles of β-carotene derived xanthophylls produced are not significantly different between lut1-2 b1 b2 and lut1-3 b1 b2 (FIG. 13). However, when one considers the total moles of hydroxylated β-rings produced in each mutant (which includes hydroxylated β-ring in zeinoxanthin), total hydroxylated β-rings are significantly reduced in lut1-2 b1 b2 and lut1-3 b1 b2 compared to b1 b2, suggesting that LUT1 also has β-ring hydroxylation activity in vivo (FIG. 13). In addition, the presence of β-carotene derived xanthophylls in the triple knockout mutant lut1-3 b1 b2 indicates a third β-hydroxylase must exist in vivo (FIG. 13).

### EXAMPLE 6

### CYP97 Homologs in Other Species

Arabidopsis LUT1 was previously designated as CYP97C1 according to the standardized cytochrome P450 nomenclature (http://www.biobase.dk/P450). The Arabidopsis genome also contains two other CYP97 family members, CYP97A3 and CYP97B3, which are 49% and 42% identical to the LUT1 protein, respectively. Interestingly, CYP97A3 (Atlg31800) is also one of the nine cytochrome P450s in Arabidopsis predicted to be chloroplast-targeted, while CYP97B3 (At4g15110) is predicted to be targeted to the mitochondria (Schuler and Werck-Reichhart, Annu. Rev. Plant Biol. 54, 629-667 (2003), herein incorporated by reference). Additional CYP97 family proteins were identified in the EST and genomic databases from a wide variety of monocots and dicots, including Arabidopsis, barley, rice, soybean, and pea (FIG. 5).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in biochemistry, molecular biology, plant biology, and chemistry or related fields are intended to be within the scope of the following claims.

1. An expression vector, comprising a nucleic acid sequence encoding a polypeptide at least 40% identical to SEQ ID NO: 1, wherein said nucleic acid encodes a protein having monooxygenase P450 activity.

2. The expression vector of claim 1, wherein the monooxygenase P450 activity is e-ring hydroxylase activity.

3. The expression vector of claim 2, wherein the monooxygenase P450 activity further comprises b-ring hydroxylase activity.

4. The expression vector of claim 1, wherein the monooxygenase P450 activity is b-ring hydroxylase activity.

5. The expression vector of claim 1, wherein said nucleic acid sequence further encodes a polypeptide comprising a cytochrome P450 molecular oxygen binding pocket conserved consensus amino acid motif corresponding to SEQ ID NO:12.

6. The expression vector of claim 5, wherein said nucleic acid sequence further encodes a polypeptide comprising a conserved transmembrane domain sequence corresponding to SEQ ID NO: 10.

7. The expression vector of claim 1, wherein said nucleic acid sequence further encodes a polypeptide comprising a conserved consensus cysteine motif corresponding to SEQ ID NO:14.

8. The expression vector of claim 7, wherein said nucleic acid sequence further encodes a polypeptide comprising a conserved N-terminal transit peptide for chloroplast-targeting corresponding to SEQ ID NO: 11.

9. The expression vector of claim 1, wherein said polypeptide at least 40% identical to SEQ ID NO: 1 is selected from the group consisting of SEQ ID NO: 1-4, 16-21, 33-39, 49-52 and 56.

10. The expression vector of claim 1, wherein said nucleic acid sequence is selected from the group consisting of SEQ ID NOs: 5-7, 22-27, 40-48, 53-55, and 57.

11. The expression vector of claim 1, wherein said vector is a eukaryotic vector.

12. The expression vector of claim 11, wherein said eukaryotic vector is a plant vector.

13. The expression vector of claim 12, wherein said plant vector comprises a T-DNA vector.

14. The expression vector of claim 1, wherein said vector is a prokaryotic vector.

15. A nucleic acid sequence encoding a polypeptide at least 40% identical to SEQ ID NO: 1 operably linked to an heterologous promoter, wherein said nucleic acid sequence encodes a protein having e-ring hydroxylase activity.

16. The promoter of claim 15, wherein said promoter is a eukaryotic promoter.

17. The promoter of claim 16, wherein said eukaryotic promoter is active in a plant.

18. An expression vector comprising a first nucleic acid sequence encoding a nucleic acid product that interferes with the expression of a second nucleic acid sequence encoding a polypeptide at least 40% identical to SEQ ID NO: 1.

19. The expression vector of claim 18, wherein said nucleic acid product that interferes is an antisense sequence.

20. The expression vector of claim 18, wherein said nucleic acid product that interferes is a dsRNA that mediates RNA interference.

21. A transgenic plant comprising a nucleic acid sequence encoding a polypeptide at least 40% identical to SEQ ID NO: 1, wherein said nucleic acid sequence encodes a protein having monooxygenase P450 activity, and wherein said nucleic acid sequence is heterologous to the plant.

22. The transgenic plant of claim 21, wherein said transgenic plant comprises one or more of the following: Brassicaceae, Poaceae, Fabaceae, Asteraceae, Solanaceae, and Volvocaceae.

23. The transgenic plant of claim 22, wherein said transgenic plant is a marigold.

24. The transgenic plant of claim 21, wherein said transgenic plant is a crop plant.

25. A transgenic plant cell comprising a nucleic acid sequence encoding a polypeptide at least 40% identical to SEQ ID NO: 1, wherein said nucleic acid sequence encodes a protein having monooxygenase P450 activity, and wherein said nucleic acid sequence is heterologous to the plant cell.

26. A transgenic plant seed comprising a nucleic acid sequence encoding a polypeptide at least 40% identical to SEQ ID NO: 1, wherein said nucleic acid sequence encodes a protein having monooxygenase P450 activity, and wherein said nucleic acid sequence is heterologous to the plant seed.

27. A transgenic plant comprising a nucleic acid encoding a polypeptide at least 40% identical to SEQ ID NO: 1 operably linked to a promoter, wherein the nucleic acid sequence encodes a protein having e-ring hydroxylase activity.

28. A method for altering the phenotype of a plant, comprising:
a) providing;
i) an expression vector comprising a nucleic acid sequence encoding a polypeptide at least 40% identical to SEQ ID NO: 1, and
ii) plant tissue; and
b) introducing said vector into said plant tissue under conditions such that expression of said nucleic acid sequence alters the phenotype of a plant.

29. A method for altering carotenoid ratios, comprising:
a) providing a vector construct comprising a nucleic acid encoding a polypeptide at least 40% identical to SEQ ID NO: 1, wherein said nucleic acid sequence encodes a protein having e-ring hydroxylase activity; and
b) producing a plant comprising the vector, wherein said plant exhibits altered carotenoid ratios.

30. A method for altering the carotenoid production of a plant, comprising:
a) providing;
i) an expression vector comprising a nucleic acid encoding a polypeptide at least 40% identical to SEQ ID NO: 1, wherein the nucleic acid sequence encodes a protein having e-ring hydroxylase activity, and
ii) plant tissue; and
b) introducing said vector into said plant tissue under conditions such that the protein encoded by the nucleic acid sequence is expressed so that the plant tissue exhibits altered carotenoid ratios.

31. A method for producing lutein, comprising:
a) providing a transgenic host cell comprising a heterologous nucleic acid sequence, wherein the heterologous nucleic acid sequence encodes a polypeptide at least 40% identical to SEQ ID NO: 1, under conditions sufficient for expression of the encoded protein; and
b) culturing said transgenic host cell under conditions such that lutein is produced.

32. A method for altering carotenoid production in a plant, comprising:
a) providing a transgenic plant comprising a heterologous nucleic acid sequence, wherein said heterologous nucleic acid sequence encodes a polypeptide at least 40% identical to SEQ ID NO: 1,
b) cultivating said transgenic plant under conditions sufficient for increasing non-hydroxylated carotenes in the plant tissue.


SEQUENCE LISTING (74 sequences)

SEQ ID NO: 1 - protein, 77 aa, Arabidopsis thaliana
LQPYAEDGSAVNMEAKFSQMTLDVIGLSLFNYNFDSLTTDSPVIEAVYTALKEAELRSTDLLPYWKIDALCKIVPRQ

SEQ ID NO: 2 - protein, 77 aa, Arabidopsis thaliana
LDAAALKGEEVEMESLFSRLTLDIIGKAVFNYDFDSLTNDTGVIEAVYTVLREAEDRSVSPIPVWDIPIWKDISPRQ

SEQ ID NO: 3 - protein, 84 aa, Arabidopsis thaliana
EKLIREKETSSGEDTIELDLEAEFSSLALDIIGLSVFNYDFGSVTKESPVIKAVYGTLFEAEHRSTFYFPYWNFPPARWIVPRQ

SEQ ID NO: 4 - protein, 539 aa, Arabidopsis thaliana
MESSLFSPSSSSYSSLFTAKPTRLLSPKPKFTFSIRSSIEKPKPKLETNSSKSQSWVSPDWLTTLTRTLSSGKNDESGIPIANAKLDDVADLLGGALFLPLYKWMNEYGPIYRLAAGPRNFVIVSDPAIAKHVLRNYPKYAKGLVAEVSEFLFGSGFAIAEGPLWTARRRAVVPSLHRRYLSVIVERVFCKCAERLVEKLQPYAEDGSAVNMEAKFSQMTLDVIGLSLFNYNFDSLTTDSPVIEAVYTALKEAELRSTDLLPYWKIDALCKIVPRQVKAEKAVTLIRETVEDLIAKCKEIVEREGERINDEEYVNDADPSILRFLLASREEVSSVQLRDDLLSMLVAGHETTGSVLTWTLYLLSKNSSALRKAQEEVDRVLEGRNPAFEDIKELKYITRCINESMRLYPHPPVLIRRAQVPDILPGNYKVNTGQDIMISVYNIHRSSEVWEKAEEFLPERFDIDGAIPNETNTDFKFIPFSGGPRKCVGDQFALMEAIVALAVFLQRLNVELVPDQTISMTTGATIHTTNGLYMKVSQR

SEQ ID NO: 5 - DNA, 2467 bp, Arabidopsis thaliana
atggagtcttcactcttttctccatcttcctcttcttactcttctctcttcactgcaaaacctacgcgtcttttatcaccaaaacccaaattcacattctccatcagatcctccattgagaaacccaaacccaaactcgagaccaattcatcgaaatcccaatcatgggtcagtcccgattggctcacaacactcactcgtaccctttcctcaggaaaaaacgacgagtcaggtataccaatcgcgaacgcgaagctcgacgatgtcgctgatctcctcggaggtgctctcttcttacctctctacaaatggatgaatgagtacggacccatttaccgtctcgctgctggtcctcgtaatttcgtaattgtgagcgacccagcgatagctaaacatgttttgaggaattatccaaagtacgctaaaggcttagtcgctgaagtctctgaatttctatttggttcgggtttcgctatcgctgaaggacctctttggacagtaatttcatctcctcctatctcaattttgaagtttttggaattgtggaagtaatgtgtgactgtcttgtatgataagtaactctaattttagggtttagattccaatcttctctattgggcttagctgaagtctgattttttacataggcgaggcgtagagcggtggttccatcgcttcacaggaggtatttgtctgtgattgtggagagagtattctgcaaatgtgcagagaggcttgttgagaagttgcagccttatgcagaagacggaagtgctgtgaatatggaagcgaagttctctcagatgacacttgatgtcattgggttgtctctttttaactacaatttcgattctttgactactgatagtcctgtcattgaagctgtttacactgctcttaaagaagctgagcttcgttctactgatcttctgccatattggaaggcaagtttcctgtgttttttctgtggtttgttgattgtgtggaacaattggattcttgttaattgagagggtttggttgtttttttcagatcgatgcattgtgtaagatagtcccgagacaggtgaaagctgaaaaggctgtaactttgataagggaaactgttgaagaccttattgctaagtgtaaagaaattgtcgaaagagaaggcgaaagaatcaatgatgaggagtatgtaaatgatgctgacccaagtatcctgcgtttcttgcttgcaagcagagaagaggtttaaacttttttccttaagtttataagcaaatttggcctttcattatcgcataatcgaagctgatgttgcattgtgagggttttcaggtatcaagtgtgcagttacgggatgatcttctctcaatgctcgtagcgggtcatgaaaccactggatctgtcctcacttggacactttatctcctaagtaaggtaccttaatgtatcttctactttgctatgctagagaatttacttggatgggagcttctctgttctcatttacctcttcaaattctctatgttcatagaactcatctgcattaaggaaagcacaagaagaagtagacagagtgttagaaggaagaaacccggctttcgaggatataaaggagttgaagtacatcactcgttgtataaacgagtcaatgcgtctctatcctcatcctcctgtaagcaatcaagctcatctctctaattattcatgaactaaattttctgattgatttgtttcctggtaggtcttgataagaagagctcaagttcctgacattcttcctgggaactataaggtcaataccggacaagacattatgatttcagtctataacatccatcgttcttccgaggtacagttctcttccttctctcgtccatagtataacataggggagcctaatccttctcttcaatgatctttgtgtggttcggatatctaaccggagtggacattcctagtattacattcatgcccacatttcttatgtgtttgttgtttgttattccaaaggtatgggaaaaagctgaggaatttctgcctgaacgattcgacatagatggcgcaatccctaacgaaacaaacactgatttcaagtaaactcagtagaacacatcttttgacacaaactactgaatcaagattagtggttttgattagggaatttaaaagatgattttcttttttcaccagattcatcccattcagtggagggcctagaaaatgtgtaggcgatcagtttgcattgatggaggcaattgtggcactcgcggtgtttcttcagcggttaaacgttgagctggttcctgatcagaccattagcatgaccacaggagcaaccatacacaccaccaatgtatgccaatgttctcacactcgagagattaatgagagtgtctgttttgtttagaatgattccaatttctctaatgctgatattttcaatttcagggattgtatatgaaggtgagccaaaggtaa

SEQ ID NO: 6 - DNA, 4170 bp, Arabidopsis thaliana
cctggtcaagagagagcagaaattgctgatacgcatctttgtcagagagaagcttcctgagttcatccacgctacagaaaagtatacactattcaaaaagaagacatacaaaaaaaacgcacactcgagagaaggataacaaacaaacaaacaaaaagagcaaaccttttgtctttcaagaagacgataatgccagctgcttcacccggtgatacttgtgctgaaatctgacctgaactttgaggccgggatgagcttggcgagctaactaaagaaggcgagtaccatggcgactgtgaagaagcttcctgaggacgagattgcccttgttgttggtcttttgatccccttcacgtacacaacaaaaaaaagaaacatcatcaactaactatctcagcttccaagttaatataatacatcacctcataaacttgaaaggtgcatttatacagcacatcattgtaaacctaatatcagtaactatgaaccctaaatcagtagctgagcaaaatctacacttgtcaattcacctcaaaaacctcaaatatatcattatagctcaacaaactacacaacaacaagatttccttaatcgctaacagacctaatgtctggatcacgcaatttcatcaataacatatgaatcagatggcaaaaaattcgcaaaatcataccagaaattgaacatcgtttctagaattcgccgactaacgaaaggaattgactttttcttgttctcttgtacttcttcttcctaccagatcgacaaaatttaacaactttacagaaacagagtttacagagtttcaaagaaaattcgatcttctctgtttcgtttcagattttgctacttgcttttaaaaggcccaaatacgaggaggcccatattaagttttgattaacaaaacgtagtcgttttatgcatcgtcgtccttatccacgcgtaacggttcgcgtttctacacagagtcaaatatttctgcacggaagcttcgaaaagaggtcatcaatggagtcttcactcttttctccatcttcctcttcttactcttctctcttcactgcaaaacctacgcgtcttttatcaccaaaacccaaattcacattctccatcagatcctccattgagaaacccaaacccaaactcgagaccaattcatcgaaatcccaatcatgggtcagtcccgattggctcacaacactcactcgtaccctttcctcaggaaaaaacgacgagtcaggtataccaatcgcgaacgcgaagctcgacgatgtcgctgatctcctcggaggtgctctcttcttacctctctacaaatggatgaatgagtacggacccatttaccgtctcgctgctggtcctcgtaatttcgtaattgtgagcgacccagcgatagctaaacatgttttgaggaattatccaaagtacgctaaaggcttagtcgctgaagtctctgaatttctatttggttcgggtttcgctatcgctgaaggacctctttggacagtaatttcatctcctcctatctcaattttgaagtttttggaattgtggaagtaatgtgtgactgtcttgtatgataagtaactctaattttagggtttagattccaatcttctctattgggcttagctgaagtctgattttttacataggcgaggcgtagagcggtggttccatcgcttcacaggaggtatttgtctgtgattgtggagagagtattctgcaaatgtgcagagaggcttgttgagaagttgcagccttatgcagaagacggaagtgctgtgaatatggaagcgaagttctctcagatgacacttgatgtcattgggttgtctctttttaactacaatttcgattctttgactactgatagtcctgtcattgaagctgtttacactgctcttaaagaagctgagcttcgttctactgatcttctgccatattggaaggcaagtttcctgtgttttttctgtggtttgttgattgtgtggaacaattggattcttgttaattgagagggtttggttgtttttttcagatcgatgcattgtgtaagatagtcccgagacaggtgaaagctgaaaaggctgtaactttgataagggaaactgttgaagaccttattgctaagtgtaaagaaattgtcgaaagagaaggcgaaagaatcaatgatgaggagtatgtaaatgatgctgacccaagtatcctgcgtttcttgcttgcaagcagagaagaggtttaaacttttttccttaagtttataagcaaatttggcctttcattatcgcataatcgaagctgatgttgcattgtgagggttttcaggtatcaagtgtgcagttacgggatgatcttctctcaatgctcgtagcgggtcatgaaaccactggatctgtcctcacttggacactttatctcctaagtaaggtaccttaatgtatcttctactttgctatgctagagaatttacttggatgggagcttctctgttctcatttacctcttcaaattctctatgttcatagaactcatctgcattaaggaaagcacaagaagaagtagacagagtgttagaaggaagaaacccggctttcgaggatataaaggagttgaagtacatcactcgttgtataaacgagtcaatgcgtctctatcctcatcctcctgtaagcaatcaagctcatctctctaattattcatgaactaaattttctgattgatttgtttcctggtaggtcttgataagaagagctcaagttcctgacattcttcctgggaactataaggtcaataccggacaagacattatgatttcagtctataacatccatcgttcttccgaggtacagttctcttccttctctcgtccatagtataacataggggagcctaatccttctcttcaatgatctttgtgtggttcggatatctaaccggagtggacattcctagtattacattcatgcccacatttcttatgtgtttgttgtttgttattccaaaggtatgggaaaaagctgaggaatttctgcctgaacgattcgacatagatggcgcaatccctaacgaaacaaacactgatttcaagtaaactcagtagaacacatcttttgacacaaactactgaatcaagattagtggttttgattagggaatttaaaagatgattttcttttttcaccagattcatcccattcagtggagggcctagaaaatgtgtaggcgatcagtttgcattgatggaggcaattgtggcactcgcggtgtttcttcagcggttaaacgttgagctggttcctgatcagaccattagcatgaccacaggagcaaccatacacaccaccaatgtatgccaatgttctcacactcgagagattaatgagagtgtctgttttgtttagaatgattccaatttctctaatgctgatattttcaatttcagggattgtatatgaaggtgagccaaaggtaaaaaccagaatttatgttttcatgataattgattggtgtgaatggacttgtttcatagtactcttgagaaataaccacaaaaaaatgaattatggaaaatagttgtaccatcggaaatgtgaattttgaacagtgagatgctagtaccattaaagtttggtaattgtgttatcatttcaaatcaaactttcatacagcttgtgtttgttctccatttttcagtaattacgaaaatcaaaattttatttttttttgaatttctaaataatagactaaggtctaaaaccatcccattccatatggaccacacgtaacattaaccttaaaaaccatataaaaccatataaaaccaatactagtgggttctatagcgatgtgtagtttcactttcacgtacgaggaagattaaaaaaaatggagaacgaaagctcagagagtagaaacagagctcgtcttgccattatggagcttgctaacatgattagcgttcccatgtctctcaatgccgccgtgcgactaggcattgccgacgccatttggaacggcggagccaattctcctctctctgccgccgagatcctccctcgcctccacctaccatctcacactaccattggtggcgaccccgagaatcttcagcgtatacttcggatgctcaccagctacggtgtcttctccgaacaccttgttggatc

SEQ ID NO: 7 - DNA, 2467 bp, Arabidopsis thaliana
atggagtcttcactcttttctccatcttcctcttcttactcttctctcttcactgcaaaacctacgcgtcttttatcaccaaaacccaaattcacattctccatcagatcctccattgagaaacccaaacccaaactcgagaccaattcatcgaaatcccaatcatgggtcagtcccgattggctcacaacactcactcgtaccctttcctcaggaaaaaacgacgagtcaggtataccaatcgcgaacgcgaagctcgacgatgtcgctgatctcctcggaggtgctctcttcttacctctctacaaatggatgaatgagtacggacccatttaccgtctcgctgctggtcctcgtaatttcgtaattgtgagcgacccagcgatagctaaacatgttttgaggaattatccaaagtacgctaaaggcttagtcgctgaagtctctgaatttctatttggttcgggtttcgctatcgctgaaggacctctttggacagtaatttcatctcctcctatctcaattttgaagtttttggaattgtggaagtaatgtgtgactgtcttgtatgataagtaactctaattttagggtttagattccaatcttctctattgggcttagctgaagtctgattttttacataggcgaggcgtagagcggtggttccatcgcttcacaggaggtatttgtctgtgattgtggagagagtattctgcaaatgtgcagagaggcttgttgagaagttgcagccttatgcagaagacggaagtgctgtgaatatggaagcgaagttctctcagatgacacttgatgtcattgggttgtctctttttaactacaatttcgattctttgactactgatagtcctgtcattgaagctgtttacactgctcttaaagaagctgagcttcgttctactgatcttctgccatattggaaggcaagtttcctgtgttttttctgtggtttgttgattgtgtggaacaattggattcttgttaattgagagggtttggttgtttttttcagatcgatgcattgtgtaagatagtcccgagacaggtgaaagctgaaaaggctgtaactttgataagggaaactgttgaagaccttattgctaagtgtaaagaaattgtcgaaagagaaggcgaaagaatcaatgatgaggagtatgtaaatgatgctgacccaagtatcctgcgtttcttgcttgcaagcagagaagaggtttaaacttttttccttaagtttataagcaaatttggcctttcattatcgcataatcgaagctgatgttgcattgtgagggttttcaggtatcaagtgtgcagttacgggatgatcttctctcaatgctcgtagcgggtcatgaaaccactggatctgtcctcacttggacactttatctcctaagtaagataccttaatgtatcttctactttgctatgctagagaatttacttggatgggagcttctctgttctcatttacctcttcaaattctctatgttcatagaactcatctgcattaaggaaagcacaagaagaagtagacagagtgttagaaggaagaaacccggctttcgaggatataaaggagttgaagtacatcactcgttgtataaacgagtcaatgcgtctctatcctcatcctcctgtaagcaatcaagctcatctctctaattattcatgaactaaattttctgattgatttgtttcctggtaggtcttgataagaagagctcaagttcctgacattcttcctgggaactataaggtcaataccggacaagacattatgatttcagtctataacatccatcgttcttccgaggtacagttctcttccttctctcgtccatagtataacataggggagcctaatccttctcttcaatgatctttgtgtggttcggatatctaaccggagtggacattcctagtattacattcatgcccacatttcttatgtgtttgttgtttgttattccaaaggtatgggaaaaagctgaggaatttctgcctgaacgattcgacatagatggcgcaatccctaacgaaacaaacactgatttcaagtaaactcagtagaacacatcttttgacacaaactactgaatcaagattagtggttttgattagggaatttaaaagatgattttcttttttcaccagattcatcccattcagtggagggcctagaaaatgtgtaggcgatcagtttgcattgatggaggcaattgtggcactcgcggtgtttcttcagcggttaaacgttgagctggttcctgatcagaccattagcatgaccacaggagcaaccatacacaccaccaatgtatgccaatgttctcacactcgagagattaatgagagtgtctgttttgtttagaatgattccaatttctctaatgctgatattttcaatttcagggattgtatatgaaggtgagccaaaggtaa

SEQ ID NO: 8-9 - skipped (2 entries)

SEQ ID NO: 10 - protein, 24 aa, Artificial Sequence
  Synthetic
LVAEVSEFLFGSGFAIAEGPLWTA

SEQ ID NO: 11 - protein, 36 aa, Artificial Sequence
  Synthetic
MESSLFSPSSSSYSSLFTAKPTRLLSPKPKFTFSIR

SEQ ID NO: 12 - protein, 6 aa, Artificial Sequence
  Synthetic
  MISC_FEATURE (1)..(1): X is alanine or glycine.
  misc_feature (3)..(3): Xaa can be any naturally occurring amino acid
  MISC_FEATURE (4)..(4): X is aspartic acid or glutamic acid.
  MISC_FEATURE (6)..(6): X is threonine or serine.
XGXXTX

SEQ ID NO: 13 - protein, 6 aa, Artificial Sequence
  Synthetic
AGHETT

SEQ ID NO: 14 - protein, 10 aa, Artificial Sequence
  Synthetic
  misc_feature (2)..(3): Xaa can be any naturally occurring amino acid
  misc_feature (5)..(7): Xaa can be any naturally occurring amino acid
  misc_feature (9)..(9): Xaa can be any naturally occurring amino acid
FXXGXXXCXG

SEQ ID NO: 15 - protein, 10 aa, Arabidopsis thaliana
FSGGPRKCVG

SEQ ID NO: 16 - protein, 561 aa, Oryza sativa
MAAAAAAAVPCVPFLCPPPPPLVSPRLRRGHVRLRLRPPRSSGGGGGGGAGGDEPPITTSWVSPDWLTALSRSVATRLGGGDDSGIPVASAKLDDVRDLLGGALFLPLFKWFREEGPVYRLAAGPRDLVVVSDPAVARHVLRGYGSRYEKGLVAEVSEFLFGSGFAIAEGALWTVRRRSVVPSLHKRFLSVMVDRVFCKCAERLVEKLETSALSGKPVNMEARFSQMTLDVIGLSLFNYNFDSLTSDSPVIDAVYTALKEAELRSTDLLPYWKIDLLCKIVPRQIKAEKAVNIIRNTVEDLITKCKKIVDAENEQIEGEEYVNEADPSILRFLLASREEVTSVQLRDDLLSMLVAGHETTGSVLTWTIYLLSKDPAALRRAQAEVDRVLQGRLPRYEDLKELKYLMRCINESMRLYPHPPVLIRRAIVDDVLPGNYKIKAGQDIMISVYNIHRSPEVWDRADDFIPERFDLEGPVPNETNTEYRFIPFSGGPRKCVGDQFALLEAIVALAVVLQKMDIELVPDQKINMTTGATIHTTNGLYMNVSLRKVDREPDFALSGSR

SEQ ID NO: 17 - protein, 545 aa, Hordeum vulgare
  misc_feature (529)..(529): Xaa can be any naturally occurring amino acid
MPAAAFASALASPPPPWAPRPSPRHASLRLPPPRSSGGGGDKPTTSWVSPDWLTSLSRSVLGRGNDDSGIPVASAKLDDVQDLLGGALFLPLFKWFREEGPVYRLAAGPRDFVIVSDPAVAKHVLRGYGTRYEKGLVAEVSEFLFGSGFAIAEGALWTVRRRAVVPSLHKRFLSVMVDKVFCKCAERLVEKLETYALSGEPVNMEARFSQMTLDVIGLSLFNYNFDSLTSDSPVIDAVYTALKEAEARSTDLLPYWQIDLLCKIVPRQIKAEKAVNTIRNTVEELIIKCKAIVDAENEQIEGEEYVNEADPSILRFLLASREEVSSLQLRDDLLSMLVAGHETTGSVLTWTIYLLSKDPVALRRAQDEVDRVLQGRLPRYEDVKELKYLMRCINESMRLYPHPPVLIRRALVDDVLPGNYKVKTGQDIMISVYNIHRSPEVWDRADEFIPERFDLEGPIPNESNTDFRFIPFSGGPRKCVGDQFALLEAIVALAIVIQKMDVQLVADQKISMTTGATIHTTNGLYMNVXLRKVEQEADLALSPSG

SEQ ID NO: 18 - protein, 362 aa, Triticum aestivum
MPAAAFASAFASPPPPWAPRPPPRHASLRLPPPRSSSNNSGGGGGDKPTTSWVSPDWLTSLSRSVLGRGNDDSGIPVASAKLDDVQDLLGGALFLPLFKWFREEGPVYRLAAGPRDFVIVSDPAVAKHVLRGYGTRYEKGLVAEVSEFLFGSGFAIAEGALWTVRRRAVVPSLHKRFLSVMVDKVFCKCAERLVEKLETYALSGEPVNMEARFSQMTLDVIGLSLFNYNFDSLTSDSPVIDAVYTALKEAEARSTDLLPYWQIDLLCKIVPRQIKAEKAVNTIRNTVEELITKCKAIVDAENEQIEGEEYVNEADPSILRFLLASREEVSSLQLRDDLLSMLVAGHETTGSVPDYRLQAQGS

SEQ ID NO: 19 - protein, 279 aa, Lycopersicon esculentum
CRCAERMVEKLLPDAISGSAVNMEAKFSQLTLDVIGLALFNYNFDSLTTDSPVIDAVYTALKEAELRSTDLLPYWQIKALCKFIPRQIKAENAVSLIRQTVEELIAKCREIVETEGERINEDEYVNDRDPSILRFLLASREEVSSLQLRDDLLSMLVAGHETTGSVLTWTAYLLSKDPSSLEKAHEEVDRVLGGRSPTYEDMKNLKFLTRCITESLRLYPHPPVLIRRAQVADVLPGNYKVNVGQDIMISVYNIHHSSKVWDRAEEFDPERFDLERSRP

SEQ ID NO: 20 - protein, 177 aa, Zea mays
LEPYALSGEPVNMEARFSQLTLDVIGLSLFNYNFDSLTTDSPVIDAVYTALKEAELRSTDLLPYWKVGFLCKIIPRQIKAENAVTIIRNTVEELIMKCKEIVEAENEQIEGEEYVNEGDPSILRFLLASRDEVSSVQLRDDLLSMLVAGHETTGSVLTWTIYLLSKDPTALRRAQDE

SEQ ID NO: 21 - protein, 208 aa, Helianthus annuus
GPRNFVIVSDPEIAKHVLRNYGSIYAKGLVAEVSEFLFGSGFAIAEGSLWTARRRAVVPSLHKKYLSVIVDRVFCKCSERLVEKLRSYARSDTSVNMEQQFSQLTLDVIGLAVFNYNFDSLTADSPVIESVYTALKEAEARSTDLLPYWKISALCKIIPRQIKAEQAVTVIRETVEELIIKCKEIVEKEGEKIDDEDYVNDATYIFIC

SEQ ID NO: 22 - DNA, 1686 bp, Oryza sativa
atggccgccgccgccgccgccgccgtcccgtgcgtaccattcctgtgcccgcctcctccgccattggtctcgccgcgtctccgccgtggccacgtccgcctccgcctgcggccgccaaggagcagcggcggtggaggcggaggcggagcggggggagacgagccgcccatcaccacctcgtgggtgagccccgactggctcacggcgctctcccgctcggtggcaacccgcctcggcgggggcgacgactcggggatccccgtcgcctccgccaagctcgacgacgtgcgggacctcctcggcggcgcgctcttcctccctctcttcaagtggttccgcgaggaaggccccgtctaccgcctcgcggcggggccgcgggatctcgtcgtcgtcagcgatcccgccgttgccaggcacgtgctgcgtgggtacggttcgaggtacgagaaggggctcgtcgccgaggtttccgagttcctcttcggctccgggttcgccatcgccgagggcgctctctggacggtgagacgtcgatcagttgtaccatctctacacaaacgatttctctcggtgatggttgacagagttttttgtaaatgtgctgagagattagtggagaagcttgagacatctgctttaagtggcaaacctgtaaatatggaagcaaggttctctcaaatgactttagatgtgattggtttgtccttgttcaattacaattttgattccctcacatcagatagccctgttattgatgctgtttacactgcactcaaggaagcagaacttcgttctacagatcttttaccatactggaagattgatttgctgtgcaagattgttcctagacaaataaaagcagaaaaggcagttaacatcatcaggaataccgttgaggacctaattaccaaatgcaagaagattgtagatgctgagaatgaacaaattgagggtgaggaatatgtaaatgaggcagaccctagcatcctgcgattcctacttgctagccgtgaagaggtaaccagtgtgcagttacgtgatgatctattgtcaatgttagttgctggtcatgaaacaacaggctctgtactgacgtggactatttatcttctcagtaaggatccagcagcgctgaggagagctcaagcagaggttgaccgtgttctacaaggtagactccccagatatgaagatctaaaagagctgaagtacttgatgcgctgtataaatgagtctatgcggctttatccacacccacctgtgttgatacggcgagccatagttgatgatgtgcttcccggaaactataagatcaaagctggtcaagatattatgatttcagtgtacaatatacacaggtcacctgaggtttgggacagagctgatgattttattcctgagagatttgatttagagggacctgttccaaatgagacaaacactgaatacagatttatcccattcagtggaggtcctcggaaatgtgttggagatcagtttgctctcttggaagcaattgtggcacttgctgttgtgttgcagaagatggacattgagcttgtgccagatcaaaaaattaacatgactactggggccacaattcatacaaccaatggcctgtatatgaatgtaagtctgcgtaaagttgacagggaacctgattttgcactcagtgggtccagatga

SEQ ID NO: 23 - DNA, 1638 bp, Hordeum vulgare
  misc_feature (1587)..(1587): n is a, c, g, or t
atgcccgccgcggcattcgcctccgcgctcgcgtctcctcctcctccatgggccccacgaccgtcccctcggcacgctagcctccgcctgcccccgccaaggagcagcggcggcggaggggacaagcccaccacgtcgtgggtcagccccgactggctcacgtcgctgtcccgctcggtgctcggccggggaaacgacgactcggggatccccgtcgcctccgccaagctcgacgacgtgcaggacctcctcgggggcgcgctcttcctcccgctcttcaagtggttccgcgaggaagggcccgtctaccgcctcgccgcggggccgcgcgacttcgtcatcgtcagcgaccccgccgtggccaagcacgtcctccgcgggtacggcacgcggtacgagaaggggctcgtcgccgaggtctccgagttcctctttggctctgggttcgccatcgccgagggagcgctctggacggtgagacgtagagcagttgtaccatctctacacaaaagatttctctcagtaatggttgataaagtgttttgtaaatgtgctgagagattggtggaaaagctcgagacatatgctttgagcggtgaacctgttaatatggaagcgagattttctcaaatgacactagatgtgattggtttgtctttgttcaactacaactttgattccctcacatcagatagtcctgttattgatgctgtttacaccgcactgaaagaagcagaggctcgttctacagatcttttaccatactggcagattgatttgctgtgcaagattgttcctagacagatcaaagcagaaaaggcagttaacacaataaggaatactgttgaagagctaattataaaatgcaaggcaatcgtagatgctgaaaatgaacagattgagggtgaagaatatgtaaatgaggcagatcctagcatcctgcgttttttacttgctagccgtgaagaggtcagcagtttgcagttacgtgatgatctattgtcaatgttagttgctggtcacgaaacaacaggctctgtactgacatggactatttatcttctcagtaaggatccagtagcactaaggagagcccaagatgaggtagatcgtgttctacaaggtagactcccaagatatgaagatgtaaaagagctgaagtacttgatgcgctgtatcaatgagtccatgcggctatacccacatcctcctgtgctgatacggcgtgcactagttgatgatgtgcttcctggaaactacaaggttaagactggtcaagatattatgatttctgtgtacaatattcacagatcacctgaggtatgggacagagcagatgaattcattccagagagatttgatttggagggtcccattccaaatgagtcaaacaccgatttcaggtttatccctttcagtggaggtcctcgaaaatgtgttggagatcagtttgctcttttagaagcaattgtggcacttgcaattgtcatacaaaagatggacgttcagcttgtggcagatcaaaaaatcagcatgaccactggggccaccatccatacaaccaatggactgtacatgaatgtaagnctgcgtaaagttgagcaagaagctgacttagcactgagtccatcaggctag

SEQ ID NO: 24 - DNA, 1086 bp, Triticum aestivum
atgcccgccgcggcattcgcctccgcgttcgcgtctcctcctcctccgtgggccccacgaccgcctcctcgccacgccagcctccgcctgcccccgccaaggagcagcagcaacaacagcggcggcggcggaggggacaagcccaccacctcgtgggtcagccccgactggctgacgtcgctgtctcgctcggtgctcggccgggggaacgacgactcggggatacccgtcgcctccgccaagctcgacgacgtgcaggacctcctcgggggcgcgctcttcctgccgctcttcaagtggttccgcgaggaagggcccgtctaccgcctcgccgcggggccgcgcgacttcgtcatcgtcagcgaccccgccgtagccaagcacgtcctccgcgggtacggcacgcggtacgagaaggggctcgtcgccgaggtctccgagttcctctttggctctgggttcgccatcgccgagggagcgctctggacggtgagacgtagagcagttgtaccatctctacacaaaagatttctctcagtaatggtcgataaagtgttctgtaaatgtgctgagagattggtggaaaagctcgagacttatgctttgagtggtgaacctgttaatatggaagcgaggttttctcaaatgacattagatgtgattggtttatccttgttcaactacaactttgattccctcacatcagatagtcctgttattgatgctgtttacactgcactcaaagaagctgaggctcgttctacagatcttttaccatactggcagatcgatttgctgtgcaagattgttcctagacagataaaagcggaaaaagcagttaacacaataaggaataccgttgaagagctaattacaaaatgcaaggcaatcgtagatgctgaaaatgaacagattgagggtgaagaatatgtaaatgaggcagatcctagcatcctgcggtttttacttgctagccgtgaagaggtcagcagtttgcagttacgtgatgatctattgtcaatgttagttgctggtcatgaaacaacaggttctgtaccagactatcgattacaagcccaaggttcc

SEQ ID NO: 25 - DNA, 839 bp, Lycopersicon esculentum
tgcagatgtgctgagagaatggtggagaaacttttacctgatgcaatttctggctctgcagtgaatatggaggcaaagttttctcaactaacacttgatgttattggccttgcactcttcaattacaattttgattcccttactactgacagtccagttattgatgcagtttacactgcactaaaagaagcagaactccgttcaactgatttgttgccatattggcagatcaaagctttatgtaagttcatcccacgacaaataaaggctgagaatgcagtgtcattaatcagacaaacagttgaagaacttattgcgaagtgcagagagattgtagaaactgagggtgagaggattaatgaagatgagtacgtgaatgatagagatccaagcatccttcgatttttgcttgctagccgtgaggaggtttcaagtttacaacttcgagatgatcttctgtcaatgctagttgctgggcatgaaaccacaggttcagttttgacttggacggcatacctgctgagtaaggacccttcctctttggaaaaagcacatgaggaagtagacagagttttgggaggacgctctccgacttatgaagatatgaagaatctcaagttcttaacacggtgcataactgagtcactcagactctatccacatccacctgtcctgataagacgagctcaagtagctgatgtcctccccgggaattacaaagtcaatgttggtcaggatataatgatttcggtatataacattcatcattcttcaaaagtatgggatagagctgaagaatttgatcctgaaagattcgacttggaaaggtcccgtcccaa

SEQ ID NO: 26 - DNA, 531 bp, Zea mays
cttgagccatatgctttgagtggggaacctgtcaatatggaagcgaggttttctcagttgacattggatgtgattggtttatcattgttcaactacaattttgattccctcacaacagatagtcctgtcattgatgctgtttatactgcactcaaagaagcagagcttcgttctacagatcttttgccatactggaaggttggtttcttgtgcaagataatcccaagacagataaaagcagagaatgcggttacgattataaggaacactgttgaagagctgattatgaagtgtaaagaaatagtggaagctgaaaatgaacagattgagggtgaggaatatgtaaacgaaggggatcctagcattctacgcttcctacttgctagccgagatgaggtaagcagtgtacaattacgtgatgatctcttgtcaatgttagttgctggtcatgaaacaacaggctctgtactgacgtggacaatctatcttctcagtaaggatccgactgcactgaggagagctcaagatgaa

SEQ ID NO: 27 - DNA, 624 bp, Helianthus annuus
gggccaagaaactttgtgattgtgagtgacccggagattgctaagcatgtgttgaggaattatgggagtatttatgctaaaggccttgttgctgaggtctctgagttcttgtttggttctggttttgccattgctgaaggctctctttggactgcaaggcgcagggctgtagttccatcacttcacaagaagtacttatcagtaatagttgatcgtgtattttgcaaatgctccgagaggcttgtcgaaaagctaagatcatacgcacgcagtgacacgtctgttaacatggagcaacagttttcgcagttaacccttgatgttatcggtctagccgtatttaactacaattttgactcacttacggccgatagtcctgtaattgaatctgtttataccgcactaaaagaagctgaagcccgttcaactgatcttttgccatattggaagataagtgcgttatgtaagattataccaagacaaataaaagccgagcaagcagttactgtaattagagaaactgtcgaagaacttattataaaatgcaaggaaatcgttgaaaaggaaggtgaaaaaatagacgatgaagattacgtaaatgatgcaacctatatcttcatctgc

SEQ ID NO: 28 - DNA, 29 bp, Artificial Sequence
  Synthetic
cttcctcttcttactcttctctcttcact

SEQ ID NO: 29 - DNA, 29 bp, Artificial Sequence
  Synthetic
aagaacgatggatgttatagactgaaatc

SEQ ID NO: 30 - DNA, 21 bp, Artificial Sequence
  Synthetic
ccgtctcgctgctggtcctcg

SEQ ID NO: 31 - DNA, 22 bp, Artificial Sequence
  Synthetic
ggatgaatgagtacggacccat

SEQ ID NO: 32 - DNA, 21 bp, Artificial Sequence
  Synthetic
gggtcgctcacaattacgaaa

SEQ ID NO: 33 - protein, 595 aa, Arabidopsis thaliana
MAMAFPLSYTPTITVKPVTYSRRSNFVVFSSSSNGRDPLEENSVPNGVKSLEKLQEEKRRAELSARIASGAFTVRKSSFPSTVKNGLSKIGIPSNVLDFMFDWTGSDQDYPKVPEAKGSIQAVRNEAFFIPLYELFLTYGGIFRLTFGPKSFLIVSDPSIAKHILKDNAKAYSKGILAEILDFVMGKGLIPADGEIWRRRRRAIVPALHQKYVAAMISLFGEASDRLCQKLDAAALKGEEVEMESLFSRLTLDIIGKAVFNYDFDSLTNDTGVIEAVYTVLREAEDRSVSPIPVWDIPIWKDISPRQRKVATSLKLINDTLDDLIATCKRMVEEEELQFHEEYMNERDPSILHFLLASGDDVSSKQLRDDLMTMLIAGHETSAAVLTWTFYLLTTEPSVVAKLQEEVDSVIGDRFPTIQDMKKLKYTTRVMNESLRLYPQPPVLIRRSIDNDILGEYPIKRGEDIFISVWNLHRSPLHWDDAEKFNPERWPLDGPNPNETNQNFSYLPFGGGPRKCIGDMFASFENVVAIAMLIRRFNFQIAPGAPPVKMTTGATIHTTEGLKLTVTKRTKPLDIPSVPILPMDTSRDEVSSALS

SEQ ID NO: 34 - protein, 632 aa, Oryza sativa
MAATSSAAAAAPPPCRLLGSGQAHLRLPPSAAAAAASARRRLLLRCAASGGNGKGGGGDGSGSDPVLEERRRRRQAELAARIASGEFTAQGPAWIAPLAVGLAKLGPPGELAAALLTKVAGGGGPEIPQAVGSMSAVTGQAFFIPLYDLFLTYGGIFRLNFGPKSFLIVSDPAIAKHILRDNSKAYSKGILAEILEFVMGTGLIPADGEIWRVRRRAIVPAMHQKYVTAMISLFGYASDRLCQKLDKAATDGEDVEMESLFSRLTLDVIGKAVFNYDFDSLSYDNGIVEAVYVTLREAEMRSTSPIPTWEIPIWKDISPRQKKVNEALALINKTLDELIDICKRLVEEEDLQFHEEYMNEQDPSILHFLLASGDDVSSKQLRDDLMTMLIAGHETSAAVLTWTFYLLSKYPNVMAKLQDEADTVLGDRLPTIEDVKKLKYTTRVINESLRLYPQPPVLIRRSIEEDMLGGYPIGRGEDIFISVWNLHHCPKHWDGADVFNPERWPLDGPNPNETNQNFSYLPFGGGPRKCVGDMFATFETVVATAMLVRRFDFQMAPGAPPVEMTTGATIHTTEGLKMTVTRRTKPPVIPNLEMKVISDSPENMSTTTSMPVSAASIASGEDQQGQVSATRI

SEQ ID NO: 35 - protein, 508 aa, Hordeum vulgare
SARGQAVGSLASVAGEAFFLPLYDLFLTYGGVFRLNFGPKSFLIVSDPDVAKHILRDNSKAYSKGILAEILEFVMGTGLIPADGEVWRVRRRAIVPALHQKYVTAMIGLFGNASDRLCQKLDKAASDGEDVEMESLFSRLTLDVIGKAVFNYDFDSLSYDNGIVEAVYVTLREAEMRSTSPIPTWEIPIWKDISPRQRKVNEALALINNILDELIATCKRMVDEEDLQFHEEYMNEKDPSILHFLLASGDDVSSKQLRDDLMTMLIAGHETSAAVLTWTFYLLSKYPNVMSKLQAEADAVLGDGLPTIDDVKKLKYTTRVINESLRLYPQPPVLIRRSLEDDMLGEYPIGKGEDIFISIWNLHRCPKHWDDADVFNPERWPLDGPNPNETNQKFSYLPFGGGPRKCVGDMFATFETVVATAMLVKRFDFQMAPGAPPVEMTTGATIHTTKGLNMTVTRRIKPPVIPNLEMKIVSDPEGSTSSTASVAVSTASIASGEGQQVEVSTSQV

SEQ ID NO: 36 - protein, 425 aa, Glycine max
GKGLIPADGEIWRVRRRAIVPALHQKYVAAMIGLFGQAADRLCQKLDAAASDGEDVEMESLFSRLTLDIIGKAVFNYDFDSLSNDTGIVEAVYTVLREAEDRSVAPIPVWEIPIWKDISPRLRKVNAALKFINDTLDDLIAICKRMVDEEELQFHEEYMNEQDPSILHFLLASGDDVSSKQLRDDLMTMLIAGHETSAAVLTWTFYLLSKEPRVMSKLQEEVDSVLGDQYPTIEDMKKLKYTTRVINESLRLYPQPPVLIRRSLEDDVLGEYPIKRGEDIFISVWNLHRSPKLWDDADKFKPERWALDGPSPNETNQNFKYLPFGGGPRKCVGDLFASYETVVALAMLMRRFNFQIAVGAPPVEMTTGATIHTTQGLKMTVTHRIKPPIVPSLQMSTLEVDPSISLSDQDEVSQKGEVYQAQAQS

SEQ ID NO: 37 - protein, 342 aa, Triticum aestivum
GCRLPQAVGSLASVAGEAFFLPLYDLFLTYGGVFRLNFGPKSFLIVSDPDVAKHILRDNSKAYSKGILAEILEFVMGTGLIPADGEVWRVRRRAIVPALHQKYVTAMIGLFGNASDRLCQKLDKAASDGEDVEMESLFSRLTLDVIGKAVFNYDFDSLSYDNGIVEAVYVTLREAEMRSTSPIPTWEIPIWKDISPRQCPKHWDDADVFNPERWPLDGPNPNETNQKFSYLPFGGGPRKCVGDMFATFETVVATAMLVKRFDFQMAPGAPPVEMTTGATIHTTKGLNMTVTRRIKPPVIPNLEMKIVSDSEGSTSSTASVAVSTASIASGEGQQVEVSTSQV

SEQ ID NO: 38 - protein, 579 aa, Lycopersicon esculentum
QFPTHHYSKSRLTLSPKFKGSVSNFTIRCSNSNGKQPESVDEGVKKVEKLLDEKRRAELSARIASGEFTVEQSGFPSLLKNGLSKLGVPKEFLEFFSRRTGNYPRIPEAKGSISAIRDEPFFMPLYELYLTYGGIFRLIFGPKSFLIVSDPSIAKHILKDNSKAYSKGILAEILDFVMGKGLIPADGEIWRVRRRAIVPALHQKYVAAMIGLFGKATDRLCKKLDVAATDGEDVEMESLFSRLTLDIIGKAVFNYDFDSLTVDTGIVEAVYTVLREAEDRSVAPIPVWELPIWKDISPKLKKVNAALKLINDTLDDLIAICKRMVDEEELQFHEEYMNEKDPSILHFLLASGDEVSSKQLRDDLMTMLIAGHETSAAVLTWTFYLLSKEPSVMAKLQDEVDSVLGDRLPTIEDLKKLRYTTRVINESLRLYPQPPVLIRRSIEEDVVGGYPIKRGEDIFISVWNLHRCPNHWEEADRFNPERWPLDGPNPNETNQNFSYLPFGGGPRKCVGDMFATFENLVAVAMLVQRFDFQMALGAPPVKMTTGATIHTTEGLKMTVTRRSRPPIVPNLEMATLEVD

SEQ ID NO: 39 - protein, 367 aa, Chlamydomonas reinhardtii
ARRRAVVPALHRKYVMSMVDMFGDCAAHGASATLDKYAASGTSLDMENFFSRLGLDIIGKAVFNYDFDSLAHDDPVIQAVYTLLREAEHRSTAPIAYWNIPGIQFVVPRQKRCQEALVLVNECLDGLIDKCKKLVEEEDAVFGEEFLSERDPSILHFLLASGDEISSKQLRDDLMTMLIAGHETTAAVLTWTLYLLSQHPEAAAAIRKEVDELLGDRKPGVEDLRALKMTTRVINEAMRLYPQPPVLIRRALQDDHFDQFTVPAGSDLFISVWNLHRSPKLWDEPDKFKPERFGPLDSPIPNEVTENFAYLPFGGGRRKCIGDQFALFEAVVALAMLMRRYEFNLDESKGTVGMTTGATIHTTNGLN

SEQ ID NO: 40 - DNA, 2057 bp, Arabidopsis thaliana
gtgatttgagtttttattttgcggtggcgttgtatggctatggcctttcctctttcttatactccgacgattactgttaaaccagtaacgtactctcggagatcgaactttgtagttttctcgtcgagttctaatggacgagatcctttagaggagaattcagtacctaatggtgtgaaaagcttggagaagcttcaagaagagaagcgtcgtgctgagttatctgctaggattgcttctggagctttcactgtacggaaatctagttttccatctacagtgaagaatggtttatctaagattggaataccaagcaatgttcttgatttcatgtttgattggactggttctgaccaagactaccccaaggttcctgaggctaaaggctcgattcaggcggtccggaacgaagctttcttcatccctttgtatgagcttttccttacttatggtggaattttcaggttgacctttgggcctaagtcattcttgatcgtgtcggatccttctattgctaaacatatattgaaggacaatgcaaaagcttactccaaggggattttagctgaaattctagattttgtgatgggaaaaggactcattcctgctgatggggagatatggcgtagacgaaggcgtgccattgttcctgcattgcatcaaaagtatgtagcagctatgattagtttattcggagaagcttcagataggctttgtcagaagcttgatgctgctgcattgaaaggggaagaagtagagatggaatcactcttctctcgtttgacacttgatattattggcaaggcggttttcaattacgactttgactcccttactaatgataccggtgtgatcgaggcagtgtacactgttctaagagaagctgaagacagaagtgtttcacctattcctgtttgggacatacccatttggaaagatatttccccacgtcagaggaaagttgctacttccttgaaattaatcaatgacacacttgatgatttgattgcaacatgcaagagaatggtagaagaagaggagttgcagtttcacgaggagtatatgaacgaaagagatcctagcatccttcactttcttttagcttcaggagatgatgtctctagtaagcagcttcgtgatgacttgatgacaatgcttatagccggacatgaaacatcggcggcagtattaacatggaccttttaccttttaacaacggaaccaagtgtagttgccaaacttcaagaagaggttgattctgtaattggagatagattcccaaccatacaagatatgaaaaagctgaaatacactactcgagtcatgaatgagtcattgagattatatccacaaccaccagtactgatccgtcgttctatagataatgatatacttggagagtatccgataaaaaggggagaggatatcttcatctcggtttggaatctacatcgaagtcctctgcattgggatgatgcagagaagttcaatcccgagagatggcctttggatggaccaaacccaaatgagacaaaccaaaacttcagttacttacctttcggtggaggaccgcggaaatgtataggcgacatgtttgcttcctttgagaatgtggtagcaatcgcaatgcttattcgaagatttaactttcagattgcaccaggagctcctccggtgaaaatgactacaggagctacaatacacaccacagaaggattgaaattgacagtaacaaagaggacaaaacctctggacataccatccgtaccgatacttccaatggatacttcacgggatgaagtttcatctgctctttcttaagtcttcatctttacaaaactgaaaacaaacaagctcagatgaagaagcaaaaatcttgtgttagaacagcaaatgttgaattgttggaacatgaccaatgctttctgattatttatctgcactgtaaaatgcagacaagtaaaatgagaagatttattattctttggaaaaaaaaaatgtttttgtctgcacagtgaagataatataacttctgggttctatgtaaaaaaaaaaac

SEQ ID NO: 41 - DNA, 1788 bp, Arabidopsis thaliana
atggctatggcctttcctctttcttatactccgacgattactgttaaaccagtaacgtactctcggagatcgaactttgtagttttctcgtcgagttctaatggacgagatcctttagaggagaattcagtacctaatggtgtgaaaagcttggagaagcttcaagaagagaagcgtcgtgctgagttatctgctaggattgcttctggagctttcactgtacggaaatctagttttccatctacagtgaagaatggtttatctaagattggaataccaagcaatgttcttgatttcatgtttgattggactggttctgaccaagactaccccaaggttcctgaggctaaaggctcgattcaggcggtccggaacgaagctttcttcatccctttgtatgagcttttccttacttatggtggaattttcaggttgacctttgggcctaagtcattcttgatcgtgtcggatccttctattgctaaacatatattgaaggacaatgcaaaagcttactccaaggggattttagctgaaattctagattttgtgatgggaaaaggactcattcctgctgatggggagatatggcgtagacgaaggcgtgccattgttcctgcattgcatcaaaagtatgtagcagctatgattagtttattcggagaagcttcagataggctttgtcagaagcttgatgctgctgcattgaaaggggaagaagtagagatggaatcactcttctctcgtttgacacttgatattattggcaaggcggttttcaattacgactttgactcccttactaatgataccggtgtgatcgaggcagtgtacactgttctaagagaagctgaagacagaagtgtttcacctattcctgtttgggacatacccatttggaaagatatttccccacgtcagaggaaagttgctacttccttgaaattaatcaatgacacacttgatgatttgattgcaacatgcaagagaatggtagaagaagaggagttgcagtttcacgaggagtatatgaacgaaagagatcctagcatccttcactttcttttagcttcaggagatgatgtctctagtaagcagcttcgtgatgacttgatgacaatgcttatagccggacatgaaacatcggcggcagtattaacatggaccttttaccttttaacaacggaaccaagtgtagttgccaaacttcaagaagaggttgattctgtaattggagatagattcccaaccatacaagatatgaaaaagctgaaatacactactcgagtcatgaatgagtcattgagattatatccacaaccaccagtactgatccgtcgttctatagataatgatatacttggagagtatccgataaaaaggggagaggatatcttcatctcggtttggaatctacatcgaagtcctctgcattgggatgatgcagagaagttcaatcccgagagatggcctttggatggaccaaacccaaatgagacaaaccaaaacttcagttacttacctttcggtggaggaccgcggaaatgtataggcgacatgtttgcttcctttgagaatgtggtagcaatcgcaatgcttattcgaagatttaactttcagattgcaccaggagctcctccggtgaaaatgactacaggagctacaatacacaccacagaaggattgaaattgacagtaacaaagaggacaaaacctctggacataccatccgtaccgatacttccaatggatacttcacgggatgaagtttcatctgctctttcttaa

SEQ ID NO: 42 - DNA, 5071 bp, Oryza sativa
atggcggctacctcctctgcggccgccgctgctccacctccgtgccgcttactcggctccggtcaggcacacctgcgccttcctccttctgctgctgctgctgctgcttcagctcgtcgccgcctgctcctccgctgcgccgcctcgggcggcaacgggaaaggcggtggtggcgacggctccggctccgacccggttcttgaggagcggcggcggcggcgccaggctgagctggcggcgcgcattgcgtccggcgagttcaccgcccaaggccccgcgtcagtgctcattctctctctctctctctctcacgttgcggccgcctttccttcctcctttgatgatctgatggagagctccctctctctttttcaggtggattgctcccctcgcggtggggcttgccaagctcggcccaccgggggagctcgccgccgcgctgctcaccaaggtcgccggtggcggcggaccggagataccgcaggcggtggggtctatgagtgcggtgacagggcaggctttcttcatcccgctctatgatctcttccttacctatggcggcatctttcgcctcaatttcggccctaaggtgatgcacaatcagaccaatttgctctccaactcggcaactcccaattttgtgttattattgatggcctaaactttgttcttttcttgttttccccagtctttcctcattgtctctgatccagctatagctaagcacatcctgagggacaactccaaggcttattccaaggttttgtgttgtcaattttggatgtagacgtgttctaggctgtgctctagaaattaacggcctgcattttgattgtggtgggtgcagggtattctggcagaaattttagagtttgtgatgggtacgggtttgatccctgctgatggggagatttggcgtgttaggaggcgcgccattgtaccagcaatgcaccagaaggttctacatcatttctgtaccaggtttagcatgatttgatcttcgggttgtgattgaactgatctgaatttcgctttgcagtacgttaccgcaatgataagtctcttcggatatgcttcagatcggctctgccagaagttggacaaggcagcaacggatggggaggatgtggagatggaatctttgttctctcgactaacactggatgtcattgggaaggcagtcttcaattatgatttcgactcattgtcttacgataatggaatagttgaggttagtattcagtcttgtactgtaattttggaattcattatacattctatttgttcatgtttgttttcttaaaattttacctttttttggattgatgatcaggcagtgtatgtgacactgcgagaagcagaaatgcggagcacttctcctataccaacttgggaaatacccatatggaaagatatttccccgcggcagaagaaggtcaatgaagctcttgcgctgataaataagactcttgatgaactaattgacatctgcaaggtgaacttcttttcttatgttctgaccttattatttattttttaaaaaaatcaaggcttttagattggctgctgttactcttgcagagattggtcgaggaagaagatctgcagtttcatgaagaatacatgaatgagcaagaccccagtatcctccactttcttttggcatctggagatgatgtatggtgtacctgcagtttaaaatattatagatctccaaacattctggtcctcacattgcttcaatttgtcttcattaggtctccagcaagcaactccgtgatgatctgatgacaatgctcattgctggccatgagacctctgcagcagtcttgacatggacattttatcttctatctaaggtatcaatatgttccgtagctgttcccaaaagaaaatatgttcagcaactcaaatcattgatgtattaatgtagcaatatgtaatgatgaatattgtatacgtcaaaccactcatgttttacctttcttggcattggtaacttgcagtatccaaatgtaatggccaaactccaagatgaggtaaattcgcttttacatttaggattgttatttttagaggcacgtgcttctacatcttacaagttgcaaatgacttgtttcactcacttatggacaggctgatactgttctaggtgaccgtttaccaacaattgaggatgtgaagaaattgaagtatactactagagtaattaacgaagtaagtgataaacagtgctacctcattaaacaatgagtatgatcactgaatgaatatgtcattcaatcaccacttttgcagtcattgagactctatccacagccaccagttttaattcgtcgctctattgaggaggatatgctgggagggtacccaattggccggtaaagaaaactctagcagaacttatttctcaagtgtaggaaatctctcctgtagtcctgttgactgttgttacaattggaataggaaaacagaaagatcatgcctagtatcacaccagtaaagttctggtgaaactgagtctaagggcctctttatttaggcttaagtttattggtttagtattttaagtcagtttttttggcttatatgatttataagccaatggatttaaagtcctaagtttaatggtggagtcatacctctatctcacataagccaaaaaaccttttccaacctagcttttgtcttaatagtgtaacagttcgctgagccttaatagtgtaatagtggtttatggcttttaaaaaaaacatgcgaaaagctgtttgtttgtttaggcttagacttttcagctcataagctggcttataagcctaaacaaagagggcctaagatatgtgcaagtataagttatctaaccaatctttttttagagaatatcttcccaatcttgtgtgatatatttttgtcttctgcttgtataccatttctggtcgtagctctagagtatttaattgtctgaattgttcctttttaaaaatttcagtacaaatttgaacttcacctataattctgataagatattttttccctttgtttcccaggggagaagacattttcatatccgtgtggaacctacatcattgcccaaagcattgggatggtgcagatgtttttaatccagaaagatggcctttggatggaccaaatccaaatgaaacaaaccaaaatttcaggttccatctctattaatgctatgaaatgcattagctctttatttggatgcaccttatcactctaattcccccattttattagtttctgctttctactacaaaaatcagtagacatttgattatgctcggtatagttgtgttcttgtttgacgagaagtttgcgtttttacatctactaacactaagttatttggtcatgcgatgcacctgttgtaattattctagagataacaaaaacaaaaactctagctgattttcgttttcttctttgatgcaaatcatcaaattttcttcatgtgattcgtattaatttagtgctaatgctggcatgtgcatagccacaatcttagaaagtgcataaggcagtcacaacaacataaagaaaaatgggacctttttcttttgaagcagtaaagatgagataactgtgatgacttgattcctaaatttatggttttgggagctaaaccacagtttatgacaatcatgttaaaatgatattcatatggctataagcaacatgtgccaaaatgctattgtactttcaaactgatagtgcttgaaagtacttttcaactttaccacatcgccagaactgttaactggttacgcagaaacagtacttggaatagtaattttgtataaactgatagtatgtcgtatgagttccttacctgtagcaagtatgtcaacagcagaaccctttgtatggatcaacaaacatcagaggtgctttgaacaaaatccttacttgattagaagacgtaacaattcgtgccatcctgagttaatgaagacttccgactggactacaaacttcttccgtgttgcagtcttgcagggagttgatgcacggcataaaacctgtgacctgcaaatcatgttaattgaaaaacaaccaacgtatcatcaactataatgtacagcaagtctcctaggcatcacactgaaacttaaaaatggtaatctgcatttacttggcacatgacatgtcccattattttgtcagcttagattgaactcactgcggaacacatctttctttcaagacaacatccaaattattgattttcagttggcatgtcaaactttttaagctccaattttaggctgtggtagctttcattctgtgtattgcggctagcatctgttagctgtcactgcctcactggctaatttaatatatttgatgaatctacatagctaaatgggactcacgtgttctgttgaattctcagttacttgccatttggtggcggaccaaggaaatgtgtaggtgacatgtttgccactttcgaggtaatttgttttagtttttgaaggatttcttcttttaatttcaaaatgtcattttaaggaaacatagcaaacttatgtatggtccagtcttactgaaccttgttgccttgagccttctgttgtctaccataaggacattatatctcatgccatgataaataatgtagtacaataactattgagcatgcaagattccaactctaataacatggatatgccggaacttgtatgcagactgtggtggcaactgcaatgcttgtcaggcgctttgattttcaaatggctccaggagctcctccggtataatttctgtctgcttcctggttcttcatagttatcaacatacagataatcactgtgaagtatcaatatgataggttgagatgacaactggagcaacgattcacacaactgaggggttgaaaatgactgttactcggaggacaaagccacctgtaatcccaaacctagagatgaaagtcatttctgattcaccagaaaacatgagtactactacatcaatgcccgtttctgctgctagtattgcttcaggagaagatcaacaagggcaagtctcagcaactcgaatctga

SEQ ID NO: 43 - DNA, 1899 bp, Oryza sativa
atggcggctacctcctctgcggccgccgctgctccacctccgtgccgcttactcggctccggtcaggcacacctgcgccttcctccttctgctgctgctgctgctgcttcagctcgtcgccgcctgctcctccgctgcgccgcctcgggcggcaacgggaaaggcggtggtggcgacggctccggctccgacccggttcttgaggagcggcggcggcggcgccaggctgagctggcggcgcgcattgcgtccggcgagttcaccgcccaaggccccgcgtggattgctcccctcgcggtggggcttgccaagctcggcccaccgggggagctcgccgccgcgctgctcaccaaggtcgccggtggcggcggaccggagataccgcaggcggtggggtctatgagtgcggtgacagggcaggctttcttcatcccgctctatgatctcttccttacctatggcggcatctttcgcctcaatttcggccctaagtctttcctcattgtctctgatccagctatagctaagcacatcctgagggacaactccaaggcttattccaagggtattctggcagaaattttagagtttgtgatgggtacgggtttgatccctgctgatggggagatttggcgtgttaggaggcgcgccattgtaccagcaatgcaccagaagtacgttaccgcaatgataagtctcttcggatatgcttcagatcggctctgccagaagttggacaaggcagcaacggatggggaggatgtggagatggaatctttgttctctcgactaacactggatgtcattgggaaggcagtcttcaattatgatttcgactcattgtcttacgataatggaatagttgaggcagtgtatgtgacactgcgagaagcagaaatgcggagcacttctcctataccaacttgggaaatacccatatggaaagatatttccccgcggcagaagaaggtcaatgaagctcttgcgctgataaataagactcttgatgaactaattgacatctgcaagagattggtcgaggaagaagatctgcagtttcatgaagaatacatgaatgagcaagaccccagtatcctccactttcttttggcatctggagatgatgtctccagcaagcaactccgtgatgatctgatgacaatgctcattgctggccatgagacctctgcagcagtcttgacatggacattttatcttctatctaagtatccaaatgtaatggccaaactccaagatgaggctgatactgttctaggtgaccgtttaccaacaattgaggatgtgaagaaattgaagtatactactagagtaattaacgaatcattgagactctatccacagccaccagttttaattcgtcgctctattgaggaggatatgctgggagggtacccaattggccggggagaagacattttcatatccgtgtggaacctacatcattgcccaaagcattgggatggtgcagatgtttttaatccagaaagatggcctttggatggaccaaatccaaatgaaacaaaccaaaatttcagttacttgccatttggtggcggaccaaggaaatgtgtaggtgacatgtttgccactttcgagactgtggtggcaactgcaatgcttgtcaggcgctttgattttcaaatggctccaggagctcctccggttgagatgacaactggagcaacgattcacacaactgaggggttgaaaatgactgttactcggaggacaaagccacctgtaatcccaaacctagagatgaaagtcatttctgattcaccagaaaacatgagtactactacatcaatgcccgtttctgctgctagtattgcttcaggagaagatcaacaagggcaagtctcagcaactcgaatctga

SEQ ID NO: 44 - DNA, 1527 bp, Hordeum vulgare
tcggcacgagggcaggccgtcgggtcgctggcttccgtcgccggggaggccttcttcctgccgctctacgacctcttcctcacctacggcggcgtcttccgcctcaacttcgggcccaagtctttcctcatcgtctctgatccggatgtagctaagcatatcctcagggacaactcaaaggcttattccaagggtatccttgcggaaatactggagtttgtgatgggcacaggtctgatcccggctgatggggaggtctggcgtgttcgacggcgtgccattgtaccagcattgcatcagaagtacgtgacagcgatgataggtctctttggaaacgcttcagaccggctctgccagaagctcgacaaggctgcttcggacggggaggatgtggagatggaatctctcttctcccgactaacgctggatgtcatcgggaaggcggtgttcaattatgattttgattcattatcttacgataatggaatagttgaggctgtgtatgtaacactgcgggaagcagaaatgcggagtacatctcctattccaacatgggaaatacccatatggaaagacatctcccctcggcagaggaaggtcaatgaagcgcttgcactgataaataatattctcgatgaactaattgctacgtgcaagaggatggtagatgaagaagatctgcagtttcatgaggaatacatgaatgagaaagaccctagtattcttcactttctattggcatctggagatgatgtgtccagcaagcagctccgtgatgacctgatgacaatgctcatagctggccatgagacctctgcagcagtcttgacatggacattttatcttctatctaagtatcccaacgtaatgtccaagctccaagctgaggctgatgctgttctaggagatggtctgccaacaattgatgatgtgaagaaactgaagtatactactcgagttattaatgaatctttgagactatacccacagccgccagttttaattcgccgctcccttgaggatgatatgctaggagagtacccgatcggcaagggagaagatatttttatatccatctggaaccttcatcgctgcccaaagcattgggatgacgcggatgttttcaatccggaaaggtggcctttggacggaccgaatccaaatgagacaaaccaaaaattcagttacttgccatttggtggcggaccaaggaaatgtgtaggtgatatgtttgctacttttgagactgtggtagcaacagcaatgcttgtcaagcgatttgattttcagatggctccaggagcacctccggtcgagatgacaaccggagcaacgattcacacaactaagggactgaacatgactgttactcggaggataaagccacctgtaattccaaacttagagatgaaaatcgtttccgatccagaaggaagcacaagttctactgcgtcagtggctgtttctactgctagtattgcatccggagaaggtcaacaagtggaggtgtcgacaagtcaagtgtga

SEQ ID NO: 45 - DNA, 1278 bp, Glycine max
gggaaagggcttatcccagctgatggtgaaatatggcgagttagacgtcgtgctatagtcccagcattgcaccagaagtatgtagcagctatgattggccttttcggacaagctgcagataggctctgccagaagctagatgctgctgcatccgatggagaagatgttgagatggaatcacttttctctcgattgaccttggacatcattggcaaggcagtattcaattatgattttgatagtttatcaaatgacactggtatagttgaggctgtttatactgtactgagagaagcagaagatcgaagtgttgctccaattccagtctgggagatcccaatatggaaagacatatcaccacgtctaaggaaggttaatgcagctctcaaattcatcaatgatacgcttgatgatctgatagcaatatgcaagagaatggtggatgaagaagagttacagtttcatgaggagtacatgaatgagcaagatccaagtattctacacttcttgttggcgtcaggagatgatgtgtcaagtaagcaacttcgtgatgacttaatgaccatgctcattgctggacatgaaacatcagctgctgttttaacttggaccttttatcttctatccaaggagccaagagtcatgtccaagctccaagaagaggttgactctgtacttggagatcaatatccaactatagaagacatgaagaaactcaaatatacaacccgagtgatcaatgagtcattgaggctttacccacaaccacctgtgttaattcgccgctctcttgaggatgatgttcttggagagtaccctataaagagaggtgaagatatctttatatctgtatggaacctgcatcgcagtccaaaactatgggatgatgctgacaagtttaaacctgaaagatgggcattagatggaccaagtcctaatgagacaaatcaaaacttcaaatatcttccgtttggtggcggaccacggaaatgtgtaggtgatttgtttgcttcatacgagacggtagtagcactcgcaatgcttatgagacgattcaactttcaaatagcagttggagctccaccggttgagatgactactggagctacaattcatacaacacaagggttgaagatgactgtaactcacagaataaaacctcctattgtgccctcattacagatgtcaactttggaagtggatccatccataagcctttctgatcaagatgaagtaagtcagaaaggcgaagtttaccaggctcaggctcagtcctaa

SEQ ID NO: 46 - DNA, 1031 bp, Triticum aestivum
ggctgcaggctgccgcaggcggtcgggtcgctggcgtccgtcgccggggaggccttcttcctgccgctctacgacctcttcctcacctacggcggcgtcttccgcctcaacttcgggcccaagtctttcctcatcgtctctgatccggatgtagctaagcatatcctgagggacaactccaaggcttattccaagggtatccttgcggaaatattggagtttgtgatgggcacaggtctgatcccggctgatggggaggtctggcgtgttcgacggcgtgccattgtaccagcattgcatcagaagtacgtgacagcgatgataggtctcttcggaaatgcttcagaccgtctgtgccagaagctggacaaggcggcatccgatggggaggatgtggagatggaatctctcttctctcgactaacgctggatgtcatcgggaaggcagtgttcaattatgattttgattcattatcttacgataatggaatagttgaggctgtgtatgtaacattacgggaagcggaaatgcggagcacatctcctattccaacttgggaaatacccatatggaaagacatctcccctcggcagagtgcccaaagcattgggacgatgcggatgttttcaatccagaaaggtggcctttggacggaccgaatccaaatgagacaaaccaaaaattcagttatttgccatttggtggcgggccaaggaaatgtgtaggcgatatgtttgctacttttgagactgtggtggcaacagcaatgcttgtcaagcgatttgattttcagatggctccaggagcacctccggtcgagatgacaactggagcaacgattcacacaactaagggactcaacatgactgttactcggaggataaagccacctgtaattccaaacttagagatgaaaatcgtttccgattcagaaggaagcacaagttctactgcgtcagtggctgtttctactgctagtattgcatccggagaaggtcaacaagtagaggtgtcgacaagtcaagtgtga

SEQ ID NO: 47 - DNA, 1737 bp, Lycopersicon esculentum
caatttccaacacaccattactctaaatctagactcactctctcacctaaattcaagggtagtgtatcaaattttacaattaggtgttctaattctaatggaaaacagcctgagtcggtagatgaaggagtcaaaaaggtggaaaagcttttagatgagaaaaggcgagctgaattatctgctcgtattgcttcaggcgaatttactgttgaacaatctggcttcccgtcattgctcaaaaatggtttgtctaaattgggtgtaccaaaggaatttcttgagttcttctctcgacgaacgggcaattatcctcgcattccagaggcaaaaggatccatcagtgctattcgggatgagccattcttcatgccgctttatgagctttaccttacttatggcggaattttccggttgatttttggtcccaagtcttttttaatagtttctgatccatcaatagccaaacacatactgaaagataattctaaggcttattctaagggtatcctagctgaaatattggactttgtgatgggaaagggacttatacctgcagatggagaaatttggcgcgtcaggcggcgtgccattgtaccagcattgcaccaaaagtacgtagcagctatgattggcttatttggaaaagcaaccgataggttgtgcaaaaagcttgatgttgctgcaactgatggagaagatgtagagatggaatcacttttctcccgtctaacattggacatcattggcaaagctgtatttaattatgattttgactctttaactgtagatactggtatcgtggaggctgtatatacagtacttagagaagcagaagatcgtagtgttgcaccaattccagtttgggagttgcctatctggaaagatatctctccgaagctaaaaaaggttaatgcagctctcaagttgattaatgacacattggatgatctgattgctatatgtaagaggatggtagacgaagaagagttgcagtttcacgaggaatacatgaatgaaaaagatcctagcatcctccatttcttgttagcatctggagatgaggtctcaagcaagcaacttcgtgatgacctcatgacaatgcttatagcgggacatgaaacatctgcagcagtgctcacatggaccttttatctgttgtccaaggaacctagtgtcatggccaagcttcaagatgaggtcgattcagttctaggggataggttaccaaccattgaagatctaaagaaactcagatacacaactcgtgtgattaatgagtctttaagactatatccacagccaccagtcttgattcgtcgttctattgaagaggacgtagttggaggttacccgattaaaaggggtgaagacattttcatttctgtttggaacttgcatcgatgcccgaatcattgggaagaagccgatagattcaatcctgagaggtggccacttgatggacctaacccaaatgagacgaaccaaaatttcagttaccttcccttcggtggtggaccaagaaaatgtgtgggagacatgtttgccacatttgagaatttagtagcagttgcaatgcttgttcaacgatttgattttcaaatggctcttggagctcctcctgttaaaatgacaactggggctaccatccacaccacagaaggattaaaaatgactgtaacacgaagatcaagacctccaatagttcccaacttggagatggcaacattagaagtagat

SEQ ID NO: 48 - DNA, 1101 bp, Chlamydomonas reinhardtii
gcgcgccgacgcgcagtggtgccagccctgcaccgcaagtacgtgatgtcgatggtggacatgttcggcgactgcgcggcgcacggcgcgtccgccacactagacaagtatgccgcctcaggcaccagcctggacatggaaaacttcttcagccggctgggtctggacatcatcggcaaggccgtgttcaactacgacttcgactcgctggcgcacgacgaccccgtcatccaggccgtgtacacgttgctgcgcgaagcggagcaccgctccacagcgcccatcgcctactggaacattcccggcatccagtttgtggtgccgcggcagaagcgctgccaggaggcgctggtgctggtaaatgagtgcctggacggcctcatcgacaagtgcaagaagctggtcgaggaggaggacgcggtgtttggggaggagttccttagcgagcgcgacccctccatcctgcacttcctcctcgcgtctggagacgagatttcctcgaagcagttgcgcgatgacctgatgactatgctgattgcggggcacgagaccaccgccgccgtgctgacgtggacgctgtacctgctgtcccaacaccccgaggcggcagcggccatccgcaaggaggtagacgagctccttggggaccgcaagcccggggtggaagacctcagagcgctcaagatgacgactcgcgtcatcaacgaggcgatgcggctctacccacagccgccagtactcattcgccgcgcgctgcaggacgaccacttcgaccagttcacggtgccggccggcagcgacctgttcatcagcgtgtggaacttgcaccgcagccctaagctgtgggacgagcccgacaagttcaagccggagcgcttcggaccgctggacagccccatccccaacgaggtgactgaaaacttcgcctacctgccctttggcggtggccgccgcaagtgcattggcgaccagttcgctttgttcgaggcggttgttgcgctggccatgctgatgcggcgatacgagttcaacctggacgagtccaaggggacagtgggcatgacaacaggtgccaccatccacaccaccaacggtctaaac

SEQ ID NO: 49 - protein, 576 aa, Arabidopsis thaliana
MAFPAAATYPTHFQGGALHLGRTDHCLFGFYPQTISSVNSRRASVSIKCQSTEPKTNGNILDNASNLLTNFLSGGSLGSMPTAEGSVSDLFGKPLFLSLYDWFLEHGGIYKLAFGPKAFVVISDPIIARHVLRENAFSYDKGVLAEILEPIMGKGLIPADLDTWKLRRRAITPAFHKLYLEAMVKVFSDCSEKMILKSEKLIREKETSSGEDTIELDLEAEFSSLALDIIGLSVFNYDFGSVTKESPVIKAVYGTLFEAEHRSTFYFPYWNFPPARWIVPRQRKFQSDLKIINDCLDGLIQNAKETRQETDVEKLQERDYTNLKDASLLRFLVDMRGVDIDDRQLRDDLMTMLIAGHETTAAVLTWAVFLLSQNPEKIRKAQAEIDAVLGQGPPTYESMKKLEYIRLIVVEVLRLFPQPPLLIRRTLKPETLPGGHKGEKEGHKVPKGTDIFISVYNLHRSPYFWDNPHDFEPERFLRTKESNGIEGWAGFDPSRSPGALYPNEIIADFAFLPFGGGPRKCIGDQFALMESTVALAMLFQKFDVELRGTPESVELVSGATIHAKNGMWCKLKRRSK

SEQ ID NO: 50 - protein, 552 aa, Pisum sativum
MVAAPISTVKLTDANLHTRFHSSSSSTPSTLSLPLSLHFHFSSHSKRFSSIRCQSVNGEKRKQSSRNVFDNASNLLTSLLSGANLGSMPIAEGAVTDLFDRPLFFSLYDWFLEHGSVYKLAFGPKAFVVVSDPIVARHILRENAFSYDKGVLADILEPIMGKGLIPADLETWKQRRRVIAPGFHTSYLEAMVQLFTSCSERTVLKVNELLEGEGRDGQKSVELDLEAEFSNLALEIIGLGVFNYDFGSVTNESPVIKAVYGTLFEAEHRSTFYIPYWKFPLARWIVPRQRKFQDDLKVINTCLDGLIRNAKESRQETDVEKLQQRDYSNLKDASLLRFLVDMRGVDVDDRQLRDDLMTMLIAGHETTAAVLTWAVFLLAQNPDKMKKAQAEVDLVLGMGKPTFELLKKLEYIRLIVVETLRLYPQPPLLIRRSLKPDVLPGGHKGDKDGYTIPAGTDVFISVYNLHRSPYFWDRPNDFEPERFLVQNNNEEVEGWAGFDPSRSPGALYPNEIISDFAFLPFGGGPRKCVGDQFALMESTVALVCCYRISMWN

SEQ ID NO: 51 - protein, 576 aa, Glycine max
MSVDTSSTLSTVTDANLHSRFHSRLVPFTHHFSLSQPKRISSIRCQSINTDKKKSSRNLLGNASNLLTDLLSGGSIGSMPIAEGAVSDLLGRPLFFSLYDWFLEHGAVYKLAFGPKAFVVVSDPIVARHILRENAFSYDKGVLADILEPIMGKGLIPADLDTWKQRRRVIAPAFHNSYLEAMVKIFTTCSERTILKFNKLLEGEGYDGPDSIELDLEAEFSSLALDIIGLGVFNYDFGSVTKESPVIKAVYGTLFEAEHRSTFYIPYWKIPLARWIVPRQRKFQDDLKVINTCLDGLIRNAKESRQETDVEKLQQRDYLNLKDASLLRFLVDMRGADVDDRQLRDDLMTMLIAGHETTAAVLTWAVFLLAQNPSKMKKAQAEVDLVLGTGRPTFESLKELQYIRLIVVEALRLYPQPPLLIRRSLKSDVLPGGHKGEKDGYAIPAGTDVFISVYNLHRSPYFWDRPDDFEPERFLVQNKNEEIEGWAGLDPSRSPGALYPNEVISDFAFLPFGGGPRKCVGDQFALMESTVALTMLLQNFDVELKGTPESVELVTGATIHTKNGLWCNLRKRSSLH

SEQ ID NO: 52 - protein, 588 aa, Oryza sativa
MAAAAAAAVPCVPFLCPPPPPLVSPRLRRGHVRLRLRPPRSSGGGFTGGGGAGGDEPPITTSWVSPDWLTALSRSVATRLGGGDDSGIPVASAKLDDVRDLLGGALFLPLFKWFREEGPVYRLAAGPRDLVVVSDPAVARHVLRGYGSRYEKGLVAEVSEFLFGSGFAIAEGALWTVRRRSVVPSLHKRFLSVMVDRVFCKCAERLVEKLETSALSGKPVNMEARFSQMTLDVIGLSLFNYNFDSLTSDSPVIDAVYTALKEAELRSTDLLPYWKIDLLCKIVPRQIKAEKAVNIIRNTVEDLITKCKKIVDAENEQIEGEEYVNEADPSILRFLLASREEVTSVQLRDDLLSMLVAGHETTGSVLTWTIYLLSKDPAALRRAQAEVDRVLQGRLPRYEDLKELKYLMRCINESMRLYPHPPVLIRRAIVDDVLPGNYKIKAGQDIMISVYNIHRSPEVWDRADDFIPERFDLEGPVPNETNTEYRFIPFSGGPRKCVGDQFALLEAIVALAVVLQKMDFTIELVPDQKINMTTGATIHTTNGLYMNVVNIGVQVDEARKHGYNSFIVYGYTLYAYISPRIWSAMPVL

SEQ ID NO: 53 - DNA, 1734 bp, Arabidopsis thaliana
atggcttttcctgccgctgctacttatcccacccatttccaaggcggcgctcttcatctgggtaggaccgatcattgcctcttcggtttctaccctcaaaccatttcctctgtgaattctcggagagcttctgtttccatcaagtgccaatctacggagccaaagacgaatggtaacatattggacaatgcgagcaaccttttgacaaattttttaagtggtggaagtttggggtcaatgcctactgctgaaggctctgtctctgatttgtttggaaagcctctctttttatctctttacgactggttcttggagcatggaggaatttataaacttgcgtttggtccaaaagcctttgttgtcatctcagatcccattattgcaaggcatgtcctccgggaaaatgctttttcttatgacaagggagttcttgctgagatcttagagccgattatgggaaaagggttaataccggctgatctagatacgtggaagttaagaagaagagctatcactcccgcattccataaattgtatctagaggccatggtcaaagtatttagtgactgttcggagaaaatgatattgaaatctgagaaactcataagggagaaagaaacttcaagcggggaggacaccattgagttggatctggaagcagaattctcgagtctggctcttgatattataggtcttagcgtgttcaactacgattttggctctgtcacaaaagagtcccctgtgatcaaggcagtttatggaactcttttcgaggcagagcatcggtctactttctacttcccttattggaactttcctccagctagatggatagttccgaggcaacgaaagttccaaagcgatctgaagattataaacgattgccttgatggcctcattcaaaatgctaaagagacaagacagcaggaaacagatgttgagaagctccaggaaagggactacactaatctcaaggatgcaagtcttttgcggttcttagtcgatatgcgcggtgttgacattgatgaccggcagctgagggatgacttgatgactatgctaattgctggtcatgagacaacagcagcagtacttacttgggctgttttccttctgtcacaaaatcctgaaaaaattaggaaagctcaagctgagattgatgctgtgcttggtcaaggtccacccacttatgaatcaatgaaaaagctcgagtacatacgactgatcgttgtagaagtccttcgtctctttcctcagccacctttgctcatcagacgcactctcaaaccagaaacattacccggtggacacaaaggggaaaaagaaggtcataaagttccaaaagggactgatatcttcatttctgtatataatctccatagatctccatacttttgggataatccccacgattttgagcctgagaggtttttaagaacaaaggagagcaatggaattgaaggatgggctggctttgatccatctcgtagccccggggcactatatccgaatgagataatagcagactttgcattcttaccatttggtggaggaccaagaaaatgcattggagaccagtttgcactaatggaatcgaccgtcgcactagctatgttgtttcagaaattcgatgtggagctgcgtggaacgccagaatctgttgaactcgtgagcggcgcaacgattcatgccaaaaatgggatgtggtgcaaactaaagagaagatcaaagtga

SEQ ID NO: 54 - DNA, 1926 bp, Pisum sativum
catcacttaccactaactgaaacttgcaagcaccattctcaacttaacaccgtcgtcaccgccatggttgccgcccctatctcaaccgtcaaacttaccgatgccaatcttcacaccagatttcattcctcttcttcttctacaccatccaccctcagtcttccactctctcttcattttcacttttcttctcactccaaacgcttttcttctatcagatgtcaatcggttaatggtgaaaagcgaaaacaaagtagtagaaatgtgtttgacaatgctagcaacctccttacaagcttgttaagtggtgcaaatttagggtccatgcccatagctgaaggtgccgtcacagatctgtttgaccggccgctgtttttctcactatatgattggttcttagagcatggttctgtgtataaactggcgtttggaccgaaagcatttgttgttgtatcagatcccattgttgcaagacatattctgcgagaaaatgcattttcttatgacaagggagtacttgctgatatcctagaaccaattatgggaaaaggactcatacctgcagaccttgagacatggaagcaaaggagaagagtgattgctccgggtttccatacctcatacttggaagctatggtacaactattcacttcatgttcagaaagaactgtgttaaaggtcaatgagcttcttgaaggagaggggcgtgatggacagaagtcagttgaattggaccttgaggcagaattttcaaatttggctcttgagattattgggctaggtgtgttcaactatgactttggttctgtcaccaatgaatctcccgttattaaggctgtctatggcactctttttgaagccgaacatagatccactttctatattccatattggaaatttccattagcaaggtggattgtgcccaggcaaaggaagtttcaggatgaccttaaagtcattaatacttgtcttgatggacttatcagaaatgcaaaagagagcaggcaggaaacagatgttgagaaactgcagcaaagggattactcaaatttgaaggatgcaagtcttctgcgtttcctagttgatatgcggggagttgatgttgatgatcgtcagttgagggatgatttaatgacaatgcttattgctggtcatgagacgacggctgcagttcttacatgggcagttttcctgctagctcaaaatcctgacaaaatgaagaaggctcaagcagaggtagatttggtgctggggatggggaagccaacttttgaattgcttaaaaagttggagtacattaggttaattgttgtggagactcttcgattatatccacaaccacctctgctgattagacgttcactcaaacctgatgttttgccaggtggacataaaggtgacaaagatggttatacaattcctgctgggactgatgtcttcatttctgtatataatctccatcgatctccatatttttgggaccgccctaatgacttcgagcctgaacgatttctagtgcaaaacaataatgaagaagttgaagggtgggctggttttgacccatctcgaagtcctggagccttgtatccaaacgagattatatcagattttgcattcttgccttttggtggtggaccacgaaaatgcgttggagaccaatttgctctcatggaatccactgtagcgctagtatgctgctacagaatttcgatgtggaactgaaggggacccctgaatcggttgaactagttactggggcaactatccataccaaaaatggattgtggtgcaatttgaggaagagatctagtttacattgacatgttaactgcaacatttttcttatgcagaatgatgtacaaaatatttatcatttaaaatgacattaacattgaatagtgtctaatacagctaaagggtatttac

SEQ ID NO: 55 - DNA, 1731 bp, Glycine max
atgagtgtcgacacttcctccaccctctccaccgtcaccgatgccaatcttcactccagatttcattctcgtcttgttccattcactcatcatttctcactttctcaacccaaacggatttcttcaatcagatgccaatcaattaataccgataagaagaaatcaagtagaaatctgctgggcaatgcaagtaacctcctcacggacttattaagtggtggaagtatagggtctatgcccatagctgaaggtgcagtctcagatctgcttggtcgacctctctttttctcactgtatgattggttcttggagcatggtgcggtgtataaacttgcctttggaccaaaagcatttgttgttgtatcagatcccatagttgctagacatattctgcgagaaaatgcattttcttatgacaagggagtacttgctgatatccttgaaccaataatgggcaaaggactcataccagcagaccttgatacttggaagcaaaggagaagagtcattgctccggctttccataactcatacttggaagctatggttaaaatattcacaacttgttcagaaagaacaatattgaagtttaataagcttcttgaaggagagggttatgatggacctgactcaattgaattggatcttgaggcagagttttctagtttggctcttgatattattgggcttggtgtgttcaactatgactttggttctgtcaccaaagaatctccagttattaaggcagtctatggcactctttttgaagctgaacacagatccactttctacattccatattggaaaattccattggcaaggtggatagtcccaaggcaaagaaagtttcaggatgacctaaaggtcatcaatacttgtcttgatggacttatcagaaatgcaaaagagagcagacaggaaacagatgttgagaaattgcagcagagggattacttaaatttgaaggatgcaagtcttctgcgtttcctggttgatatgcggggagctgatgttgatgatcgtcagttgagggatgatttaatgacaatgcttattgccggtcatgaaacaacggctgcagttcttacttgggcagttttcctcctagctcaaaatcctagcaaaatgaagaaggctcaagcagaggtagatttggtgctgggtacggggaggccaacttttgaatcacttaaggaattgcagtacattagattgattgttgtggaggctcttcgtttatacccccaaccacctttgctgattagacgttcactcaaatctgatgttttaccaggtgggcacaaaggtgaaaaagatggttatgcaattcctgctgggactgatgtcttcatttctgtatataatctccatagatctccatatttttgggaccgccctgatgacttcgaaccagagagatttcttgtgcaaaacaagaatgaagaaattgaaggatgggctggtcttgatccatctcgaagtcccggagccttgtatccgaacgaggttatatcggattttgcattcttaccttttggtggcggaccacgaaaatgtgttggggaccaatttgctctgatggagtccactgtagcgttgactatgctgctccagaattttgacgtggaactaaaagggacccctgaatcggtggaactagttactggggcaactattcataccaaaaatggaatgtggtgcagattgaagaagagatctaatttacgttga

SEQ ID NO: 56 - protein, 659 aa, Skeletonema costatum
MASYESDLLSTWDEDPSLQKGFDWEIEKLRRYFAGLRQTPDGRWVRKSTLFEFLVTNSPSKVVGVGPDGERYESPPKPVNIFDVGVLVGKNTLTWLGFGPNLGMAAVPDAVIQKYEGSFFTFIKGALGGDLQTLAGGPLFLLLAKYYTDHGPIFNLSFGPKSFLVISDPVMARHILRDSSPEQYCKGMLAEILEPIMGDGLIPADPKIWKVRRRAVVPGFHKKWLNSMIGLFGDCGDRLVDDLEKRSTSDKPVIDMEERFCSVTLDIIGKAVFNYDFGSVTKESPIVKAVYRVLREAEHRSSSFIPYWNLPYAEKWMVGQVEFRKDMGMLDDILAKLINRAVETRQEATVEELEERETSDDPSLLRFLVDMRGEDLTSKVLRDDLMTMLIAGHETTAAMLTWTMFGLVSNDPGMMKEIQAEVRTVMGNKSRPDYDDVVAMKKLRYALIEALRLYPEPPVLIRRARQEDTLPPGGTGLSGGVKVLRGTDIFISTWNLHRAPEYWENADKYDPTRWERPFKNPGVKGWNGYDPEKQSSQSLYPNEITSDYAFLPFGAGKRKCIGDQFAMLEASVTLSMIMNKFDFTLVGTPEDVGMKTGATIHTMNGLNMMVSPRSETNPIPGTNEWWTKQHLMRGLSSTGRPYTSDEDAAWTTSANGMRP

SEQ ID NO: 57 - DNA, 1980 bp, Skeletonema costatum
atggcctcctacgagagtgatctgctctcaacatgggatgaagatccatcgctgcaaaaggggtttgactgggagattgaaaagctccgtcggtactttgccggactgcgtcaaacaccagacgggcgatgggtgcgcaagtcgacactgtttgagtttcttgtgacaaactctccaagtaaagtagttggggtaggtccggatggggaacggtatgaaagccctccgaaaccagtcaatatcttcgatgtgggagtgttagtcggtaagaatacactcacttggttgggatttggaccgaatttgggtatggccgcggtacccgatgcagtcattcaaaagtatgagggtagcttcttcacctttatcaagggagcattggggggtgatttgcaaactttggcgggtggtcctttgttcttattgcttgccaagtattatacggatcatggacccattttcaacttgagttttggaccaaagagctttttggtgatttcggatcctgttatggcgaggcatattttgagggatagttcaccggagcagtattgtaagggaatgcttgcggagattttggaaccgatcatgggtgatggattgattcctgcagatccaaagatttggaaggttcgtcgaagagctgtcgtacctggtttccacaaaaagtggctgaacagcatgattggtttgttcggagactgtggtgatcgtctcgttgacgatctagaaaagcgttctacttcagataaacctgtaattgacatggaagaacgattctgttccgtcacactcgatatcatcggtaaggcagtattcaactatgattttggatcagtgacaaaggaatcacctattgtaaaggcagtatacagagtgttacgtgaggcggagcacagatcatcttcgttcatcccctactggaacttgccttatgctgagaaatggatggtaggacaggttgaattccgcaaagatatgggaatgcttgacgatatcttggcaaaactgatcaatcgtgctgttgagactaggcaagaagctactgtcgaagagttggaagagagagaaacaagcgatgatccgagtctcttaaggttcctagttgatatgaggggagaagatttaacgagtaaagtgttgagagatgatttgatgacaatgcttattgcaggacatgaaacaacagcggcaatgctgacgtggacaatgtttgggctagtaagcaacgatcctggcatgatgaaggaaatccaggcagaagttcgaactgtcatgggcaataagtctcgaccagattacgatgatgttgtggcgatgaaaaagttgaggtatgctttgattgaagcacttcgattatatcccgagccacccgtgttgattcgcagggcaaggcaagaggacactcttccaccaggtggtacgggtctttctggaggtgtcaaagtattgcgtggaacagatatctttatttctacttggaaccttcaccgcgctccagaatactgggagaatgcagacaaatatgaccctactcgatgggagcgtccgttcaaaaacccaggtgttaagggttggaatggatatgatccggaaaaacaatcatctcaatcactttatcctaacgagataacgtcagactatgctttccttccttttggtgctgggaagagaaaatgtatcggggatcagtttgctatgctcgaggcttcggttacactatcgatgattatgaataaatttgacttcacgttggtcggtacccctgaagatgtcggcatgaagaccggagcaactattcataccatgaatgggctcaacatgatggtcagccctcgatcagagacaaacccgattccagggacaaatgagtggtggacgaaacaacatctaatgagaggtttgagttctactggaagaccatacacttccgatgaagatgccgcgtggacgacatccgctaatggcatgagaccgtga

SEQ ID NO: 58 - skipped

SEQ ID NO: 59 - DNA, 18 bp, Artificial Sequence
  Synthetic
cacacacacacacacaca

SEQ ID NO: 60 - protein, 77 aa, Oryza sativa
LETSALSGKPVNMEARFSQMTLDVIGLSLFNYNFDSLTSDSPVIDAVYTALKEAELRSTDLLPYWKIDLLCKIVPRQ

SEQ ID NO: 61 - protein, 77 aa, Zea mays
LEPYALSGEPVNMEARFSQLTLDVIGLSLFNYNFDSLTTDSPVIDAVYTALKEAELRSTDLLPYWKVGFLCKIIPRQ

SEQ ID NO: 62 - protein, 77 aa, Hordeum vulgare
LETYALSGEPVNMEARFSQMTLDVIGLSLFNYNFDSLTSDSPVIDAVYTALKEAEARSTDLLPYWQIDLLCKIVPRQ

SEQ ID NO: 63 - protein, 77 aa, Triticum aestivum; identical to SEQ ID NO: 62

SEQ ID NO: 64 - protein, 77 aa, Arabidopsis thaliana; identical to SEQ ID NO: 1

SEQ ID NO: 65 - protein, 77 aa, Helianthus annuus
LRSYARSDTSVNMEQQFSQLTLDVIGLAVFNYNFDSLTADSPVIESVYTALKEAEARSTDLLPYWKISALCKIIPRQ

SEQ ID NO: 66 - protein, 77 aa, Lycopersicon esculentum
LLPDAISGSAVNMEAKFSQLTLDVIGLALFNYNFDSLTTDSPVIDAVYTALKEAELRSTDLLPYWQIKALCKFIPRQ

SEQ ID NO: 67 - protein, 77 aa, Hordeum vulgare
LDKAASDGEDVEMESLFSRLTLDVIGKAVFNYDFDSLSYDNGIVEAVYVTLREAEMRSTSPIPTWEIPIWKDISPRQ

SEQ ID NO: 68 - protein, 77 aa, Triticum aestivum; identical to SEQ ID NO: 67

SEQ ID NO: 69 - protein, 77 aa, Oryza sativa
LDKAATDGEDVEMESLFSRLTLDVIGKAVFNYDFDSLSYDNGIVEAVYVTLREAEMRSTSPIPTWEIPIWKDISPRQ

SEQ ID NO: 70 - protein, 77 aa, Glycine max
LDAAASDGEDVEMESLFSRLTLDIIGKAVFNYDFDSLSNDTGIVEAVYTVLREAEDRSVAPIPVWEIPIWKDISPRL

SEQ ID NO: 71 - protein, 77 aa, Lycopersicon esculentum
LDVAATDGEDVEMESLFSRLTLDIIGKAVFNYDFDSLTVDTGIVEAVYTVLREAEDRSVAPIPVWELPIWKDISPKL

SEQ ID NO: 72 - protein, 77 aa, Arabidopsis thaliana; identical to SEQ ID NO: 2

SEQ ID NO: 73 - protein, 77 aa, Chlamydomonas reinhardtii
LDKYAASGTSLDMENFFSRLGLDIIGKAVFNYDFDSLAHDDPVIQAVYTLLREAEHRSTAPIAYWNIPGIQFVVPRQ

SEQ ID NO: 74 - protein, 85 aa, Arabidopsis thaliana
EKLIREKETSSGEDTIELDLEAEFSSLALDIIGLSVFNYDFGSVTKESPVIKAVYGTLFEAEHRSTFYFPYWNFPPARWIVPRQR